(19) United States
(12) Patent Application Publication
(10) Pub. No.: US 2010/0069356 A1
(43) Pub. Date: Mar. 18, 2010

(54) DIBENZOTHIAZEPINE MODULATORS OF DOPAMINE, ALPHA ADRENERGIC, AND SEROTONIN RECEPTORS

(75) Inventors: Thomas G. Gant, Carlsbad, CA (US); Sepehr Sarshar, Cardiff by the Sea, CA (US)

Correspondence Address: GLOBAL PATENT GROUP - APX, 10411 Clayton Road, Suite 304, ST. LOUIS, MO, 63131, US

(73) Assignee: AUSPEX PHARMACEUTICALS, INC., Vista, CA (US)

(21) Appl. No.: 12/467,124

(22) Filed: May 15, 2009

Related U.S. Application Data
(60) Provisional application No. 61/097,595, filed on Sep. 17, 2008.

(51) Int. Cl. A61K 31/554; C07D 281/18; A61P 25/28; A61P 25/18; A61P 25/22; A61P 25/24; A61P 25/16

(52) U.S. Cl. 514/211.09; 540/468

(57) ABSTRACT

The present invention relates to new dibenzothiazepine modulators of D1 receptors, D2 receptors, alpha-1 adrenergic receptors, alpha-2 adrenergic receptors, H1 receptors, 5-HT1A receptors, and/or 5-HT2 receptors, pharmaceutical compositions thereof, and methods of use thereof.

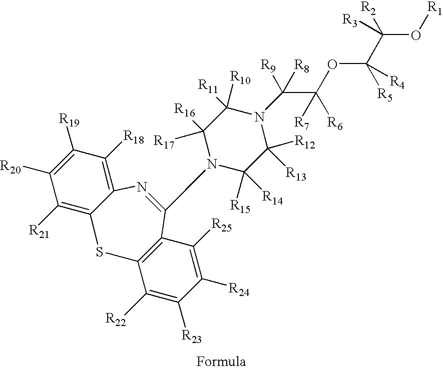

This application claims the benefit of priority of U.S. provisional application No. 61/097,595, filed Sep. 17, 2008, the disclosure of which is hereby incorporated by reference as if written herein in its entirety.

Disclosed herein are new dibenzothiazepine compounds and compositions and their application as pharmaceuticals for the treatment of disorders. Methods of modulation of D1 receptor, D2 receptor, alpha-1 adrenergic receptor, alpha-2 adrenergic receptor, H1 receptor, 5-HT1A receptor, and/or 5-HT2 receptor activity in a subject are also provided for the treatment of disorders such as schizophrenia, schizoaffective disorders, mania (manic disorder), bipolar I disorder, bipolar II disorder, depression associated with bipolar disorders, unipolar depression, Alzheimer's disease, dementia, Parkinson's disease, alcoholism, substance-related disorders, generalized agitation, generalized anxiety, anxiety disorders, anxiety neuroses, major depression (major depressive disorder), borderline personality disorder, post-traumatic stress disorder, primary insomnia, anorexia nervosa, social phobia, manic-depressive psychoses, mood disorders, psychotic disorders, psychosis, fibromyalgia, Tourette's syndrome and obsessive-compulsive disorder.

Quetiapine (Seroquel, ZD-5077, ZM-204636, FK-949, M-236303, ICI-214227, Zeneca 203636, ICI-204636, CAS #11974-72-2), 2-(2-(4-dibenzo[b,f][1,4]thiazepine-11-yl-1-piperazinyl)ethoxy)ethanol, is a D1 receptor, D2 receptor, alpha-1 adrenergic receptor, alpha-2 adrenergic receptor, H1 receptor, 5-HT1A receptor, and 5-HT2 receptor antagonist. Quetiapine is commonly prescribed for the treatment of schizophrenia and bipolar disorder. See, e.g., Dando et al., Drugs 2005, 65(17), 2533-51; Dev et al., Drug Safety 2000, 23(4), 295-307; Keating et al., Drugs 2007, 67(7), 1077-95; Khouzam et al., Expert Rev. Neurotherapeautics 2006, 6(2); and Drug Report for Quetiapine, Thompson Investigational Drug Database (Aug. 12, 2008). Quetiapine has also shown promise in treating psychotic disorders (Gerlach et al., Pharmacopsychiatry 2007, 40(2), 72-76); generalized anxiety disorder (Vaishnavi et al., Progress in Neuro-Psychopharmacology & Biological Psychiatry 2007, 31(7), 1464-1469); panic disorder (Onur et al., Klinik Psikofarmakoloji Bulteni 2007, 17(4), 203-206); major depressive disorder (Dannlowski et al., Human Psychopharmacology 2008, 23(7), 587-593; Daly et al., Neuropsychiatric Disease and Treatment 2007, 3(6), 855-867); obsessive-compulsive disorder (Savas et al., Clinical Drug Investigation 2008, 28(7), 439-442); post-traumatic stress disorder (WO 2009015236 A1); Parkinson disease (Zahodne et al., Drugs & Aging 2008, 25(8), 665-682); Alzheimer disease (Xu et al., FEBS Journal 2008, 275(14), 3718-3728; Rocca et al., Psychiatry and Clinical Neurosciences 2007, 61(6), 622-629); restless leg syndrome; dementia (Lan et al., Zhonghua Hanghai Yixue Yu Gaoqiya Yixue Zazhi 2008, 15(1), 34-36; Wen et al., Shiyong Yixue Zazhi 2007, 23(17), 2750-2752); autism (Stachnik et al., Annals of Pharmacotherapy 2007, 41(4), 626-634); alcoholism (Martinotti et al., Human Psychopharmacology 2008, 23(5), 417-424); personality disorders (Van den Eynde et al., Journal of Clinical Psychopharmacology 2008, 28(2), 147-155); fibromyalgia (Hidalgo et al., Progress in Neuro-Psychopharmacology & Biological Psychiatry 2007, 31(1), 71-77); and Tourette's syndrome (Copur et al., Clinical Drug Investigation 2007, 27(2), 123-130).

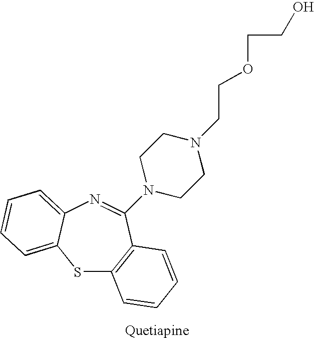

Quetiapine is subject to extensive hepatic metabolism, predominantly through CYP3A4 and CYP2D6 oxidation. Quetiapine metabolites include quetiapine sulfoxide, N-dealkyl quetiapine, and O-dealkyl quetiapine (formed by CYP3A4 oxidation), and 7-hydroxyl quetiapine and 7-hydroxyl N-dealkyl quietapine (formed in part by CYP2D6 oxidation) (Li et al., Methods Find. Exp. Clin. Pharmacol. 2005, 27(2), 83-86; Keating et al., Drugs 2007, 67(7), 1077-95; Hasselstrom et al., Drug metab. & Drug Interactions 2006, 21(3-4), 187-211). N-dealkylation, O-dealkylation, and 7-hydroxylation, as well as other metabolic transformations, occur in part through polymorphically-expressed enzymes such as CYP2D6. Quetiapine has a short half-life of ˜6 hours, and must be taken three times daily. Abruptly stopping treatment with quetiapine can lead to withdrawal or discontinuation syndrome.

### Deuterium Kinetic Isotope Effect

In order to eliminate foreign substances such as therapeutic agents, the animal body expresses various enzymes, such as the cytochrome P₄₅₀ enzymes (CYPs), esterases, proteases, reductases, dehydrogenases, and monoamine oxidases, to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT}. The Arrhenius equation states that, at a given temperature, the rate of a chemical reaction depends exponentially on the activation energy (E_{act}).

The transition state in a reaction is a short lived state along the reaction pathway during which the original bonds have stretched to their limit. By definition, the activation energy E_{act} for a reaction is the energy required to reach the transition state of that reaction. Once the transition state is reached, the molecules can either revert to the original reactants, or form new bonds giving rise to reaction products. A catalyst facilitates a reaction process by lowering the activation energy leading to a transition state. Enzymes are examples of biological catalysts.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (¹H), a C-D bond is stronger than the corresponding C—¹H bond. If a C—¹H bond is broken during a rate-determining step in a chemical reaction (i.e. the step with the highest transition state energy), then substituting a deuterium for that protium will cause a decrease in the reaction rate. This phenomenon is known as the Deuterium Kinetic Isotope Effect (DKIE). The magnitude of the DKIE can be expressed as the ratio between the rates of a given reaction in which a C—¹H bond is broken, and the same reaction where deuterium is substituted for protium. The DKIE can range from about 1 (no isotope effect) to very large numbers, such as 50 or more. Substitution of tritium for hydrogen results in yet a stronger bond than deuterium and gives numerically larger isotope effects

Deuterium (²H or D) is a stable and non-radioactive isotope of hydrogen which has approximately twice the mass of protium (¹H), the most common isotope of hydrogen. Deuterium oxide (D₂O or “heavy water”) looks and tastes like H₂O, but has different physical properties.

When pure D₂O is given to rodents, it is readily absorbed. The quantity of deuterium required to induce toxicity is extremely high. When about 0-15% of the body water has been replaced by D₂O, animals are healthy but are unable to gain weight as fast as the control (untreated) group. When about 15-20% of the body water has been replaced with D₂O, the animals become excitable. When about 20-25% of the body water has been replaced with D₂O, the animals become so excitable that they go into frequent convulsions when stimulated. Skin lesions, ulcers on the paws and muzzles, and necrosis of the tails appear. The animals also become very aggressive. When about 30% of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O Studies have also shown that the use of D₂O can delay the growth of cancer cells and enhance the cytotoxicity of certain antineoplastic agents.

Deuteration of pharmaceuticals to improve pharmacokinetics (PK), pharmacodynamics (PD), and toxicity profiles has been demonstrated previously with some classes of drugs. For example, the DKIE was used to decrease the hepatotoxicity of halothane, presumably by limiting the production of reactive species such as trifluoroacetyl chloride. However, this method may not be applicable to all drug classes. For example, deuterium incorporation can lead to metabolic switching. Metabolic switching occurs when xenogens, sequestered by Phase I enzymes, bind transiently and re-bind in a variety of conformations prior to the chemical reaction (e.g., oxidation). Metabolic switching is enabled by the relatively vast size of binding pockets in many Phase I enzymes and the promiscuous nature of many metabolic reactions. Metabolic switching can lead to different proportions of known metabolites as well as altogether new metabolites. This new metabolic profile may impart more or less toxicity. Such pitfalls are non-obvious and are not predictable a priori for any drug class.

Quetiapine is a D1 receptor, D2 receptor, alpha-1 adrenergic receptor, alpha-2 adrenergic receptor, H1 receptor, 5-HT1A receptor, and 5-HT2 receptor antagonist. The carbon-hydrogen bonds of quetiapine contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Deuterium Kinetic Isotope Effect (DKIE) that could effect the pharmacokinetic, pharmacologic and/or toxicologic profiles of such quetiapine in comparison with the compound having naturally occurring levels of deuterium.

Based on discoveries made in our laboratory, as well as considering the literature, quetiapine is metabolized in humans at the methylene group alpha to the piperazine ring, the methylene groups alpha to the ether oxygen, and at the 7-position on the tricyclic ring. The current approach has the potential to prevent metabolism at these site. Other sites on the molecule may also undergo transformations leading to metabolites with as-yet-unknown pharmacology/toxicology. Limiting the production of these metabolites has the potential to decrease the danger of the administration of such drugs and may even allow increased dosage and/or increased efficacy. All of these transformations can occur through polymorphically-expressed enzymes, exacerbating interpatient variability. Further, some disorders are best treated when the subject is medicated around the clock or for an extended period of time. For all of the foregoing reasons, a medicine with a longer half-life may result in greater efficacy and cost savings. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not. The deuteration approach has the strong potential to slow the metabolism of quetiapine and attenuate interpatient variability.

Novel compounds and pharmaceutical compositions, certain of which have been found to modulate D1 receptors, D2 receptors, alpha-1 adrenergic receptors, alpha-2 adrenergic receptors, H1 receptors, 5-HT1A receptors, and/or 5-HT2 receptors have been discovered, together with methods of synthesizing and using the compounds, including methods for the treatment of D1 receptor-mediated disorders, D2 receptor-mediated disorders, alpha-1 adrenergic receptor-mediated disorders, alpha-2 adrenergic receptor-mediated disorders, H1 receptor-mediated disorders, 5-HT1A receptor-mediated disorders, and/or 5-HT2 receptor-mediated disorders in a patient by administering the compounds.

In certain embodiments of the present invention, compounds have structural Formula I:

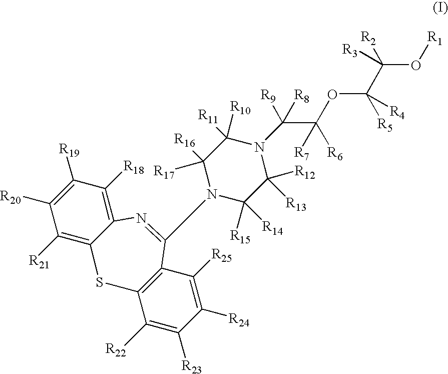

or a salt, solvate, or prodrug thereof, wherein:

R₁-R₂₅ are independently selected from the group consisting of hydrogen and deuterium; and

at least one of R₁-R₂₅ is deuterium.

Certain compounds disclosed herein may possess useful D1 receptor, D2 eceptor, alpha-1 adrenergic receptor, alpha-2 adrenergic receptor, H1 receptor, 5-HT1A receptor, and/or 5-HT2 receptor modulating activity, and may be used in the treatment or prophylaxis of a disorder in which D1 receptors, D2 receptors, alpha-1 adrenergic receptors, alpha-2 adrenergic receptors, H1 receptors, 5-HT1A receptors, and/or 5-HT2 receptors play an active role. Thus, certain embodiments also provide pharmaceutical compositions comprising one or more compounds disclosed herein together with a pharmaceutically acceptable carrier, as well as methods of making and using the compounds and compositions. Certain embodiments provide methods for modulating D1 receptors, D2 receptors, alpha-1 adrenergic receptors, alpha-2 adrenergic receptors, H1 receptors, 5-HT1A receptors, and/or 5-HT2 receptors. Other embodiments provide methods for treating a D1 receptor-mediated disorder, a D2 receptor-mediated disorder, an alpha-1 adrenergic receptor-mediated disorder, an alpha-2 adrenergic receptor-mediated disorder, a H1 receptor-mediated disorder, a 5-HT1A receptor-mediated disorder, and/or a 5-HT2 receptor-mediated disorder in a patient in need of such treatment, comprising administering to said patient a therapeutically effective amount of a compound or composition according to the present invention. Also provided is the use of certain compounds disclosed herein for use in the manufacture of a medicament for the prevention or treatment of a disorder ameliorated by the modulation of D1 receptors, D2 receptors, alpha-1 adrenergic receptors, alpha-2 adrenergic receptors, H1 receptors, 5-HT1A receptors, and/or 5-HT2 receptors.

The compounds as disclosed herein may also contain less prevalent isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, the compound disclosed herein may expose a patient to a maximum of about 0.000005% D₂O or about 0.00001% DHO, assuming that all of the C-D bonds in the compound as disclosed herein are metabolized and released as D₂O or DHO. In certain embodiments, the levels of D₂O shown to cause toxicity in animals is much greater than even the maximum limit of exposure caused by administration of the deuterium enriched compound as disclosed herein. Thus, in certain embodiments, the deuterium-enriched compound disclosed herein should not cause any additional toxicity due to the formation of D₂O or DHO upon drug metabolism.

In certain embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

In certain embodiments, if R₂-R₅ are deuterium, then at least one of R₁ and R₆-R₂₅ is deuterium.

In other embodiments, if R₂-R₉ are deuterium, then at least one of R₁ and R₁₀-R₂₅ is deuterium.

In other embodiments, if R₆-R₉ are deuterium, then at least one of R₁-R₅ and R₁₀-R₂₅ is deuterium.

In certain embodiments of the present invention, compounds have structural Formula II:

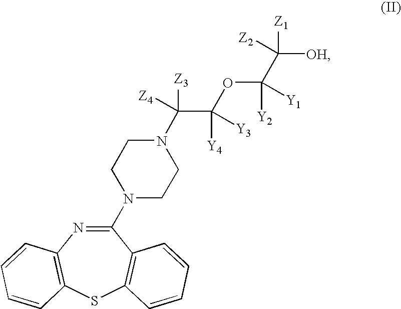

or a salt, hydrate, or solvate thereof, wherein:
   each Z is independently selected from hydrogen or deuterium;
   each Y is independently selected from hydrogen or deuterium; and
   at least one Z is deuterium.

In further embodiments, Y₁ and Y₂ are each deuterium.

In further embodiments, Z₁ and Z₂ are each deuterium.

In yet further embodiments, Z₃ and Z₄ are each deuterium.

In certain embodiments, compounds have a structure selected from the group consisting of:

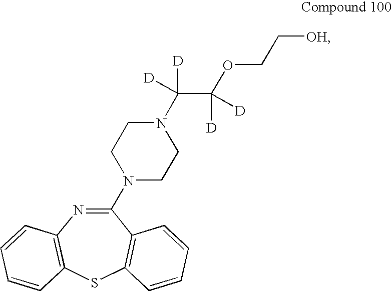

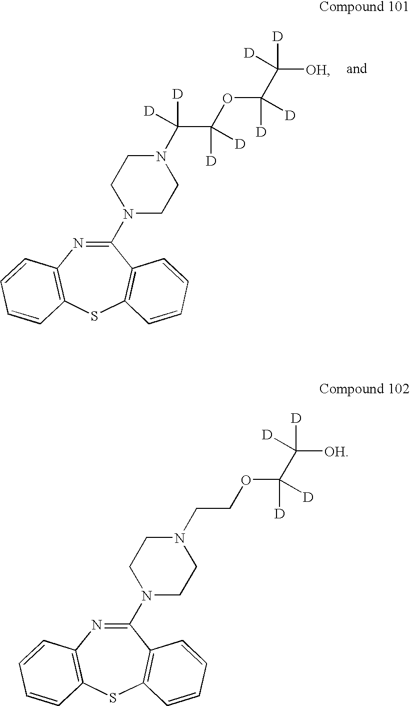

In certain embodiments, any atom not designated as deuterium is present at its natural isotopic abundance.

In certain embodiments, a pyrogen-free composition comprises a compound as described herein; and an acceptable carrier.

In certain embodiments, a composition as described herein is formulated for pharmaceutical administration, wherein the carrier is a pharmaceutically acceptable carrier.

In further embodiments, a composition as described herein; further comprises a second therapeutic agent useful in the treatment of a disease or condition selected from schizophrenia, schizoaffective disorders, mania (manic disorder), bipolar I disorder, bipolar II disorder, depression associated with bipolar disorders, unipolar depression, Alzheimer's disease, dementia, Parkinson's disease, alcoholism, substance-related disorders, generalized agitation, generalized anxiety, anxiety disorders, anxiety neuroses, major depression (major depressive disorder), borderline personality disorder, post-traumatic stress disorder, primary insomnia, anorexia nervosa, social phobia, manic-depressive psychoses, mood disorders, psychotic disorders, psychosis, fibromyalgia, Tourette's syndrome and obsessive-compulsive disorder.

In further embodiments, the second therapeutic agent is selected from sabcomeline; a nicotine acetylcholine alpha 7 receptor agonist; moclobemide; brofaromine; befloxatone; toloxatone; gluoxetine; citalopram; excitalopram; fluvoxamine; sertraline; paroxetine; a dopamine D1 antagonist; zolmitriptan; divalproex; lithium; and guanfacine.

In yet further embodiments, the second therapeutic agent is selected from divalproex and lithium.

In certain embodiments, a method of modulating the activity of one or more of: serotonergic 5HT1A or 5HT2 receptors, dopaminergic D1 or D2 receptor, histaminergic H1 receptors, or adrenergic α1 or α2 receptors in a cell comprises contacting the cell with a compound as described herein.

In other embodiments, a method of treating a subject suffering from or susceptible to a disorder selected from schizophrenia, schizoaffective disorders, mania (manic disorder), bipolar I disorder, bipolar II disorder, depression associated with bipolar disorders, unipolar depression, Alzheimer's disease, dementia, Parkinson's disease, alcoholism, substance-related disorders, generalized agitation, generalized anxiety, anxiety disorders, anxiety neuroses, major depression (major depressive disorder), borderline personality disorder, post-traumatic stress disorder, primary insomnia, anorexia nervosa, social phobia, manic-depressive psychoses, mood disorders, psychotic disorders, psychosis, fibromyalgia, Tourette's syndrome and obsessive-compulsive disorder, comprises the step of administering to the patient mammal in need thereof with a composition as described herein.

In other embodiments, the subject is suffering from or susceptible to schizophrenia or bipolar I disorder.

In further embodiments, the method as described herein comprises the additional step of co-administering to the patient in need thereof a second therapeutic agent selected from sabcomeline; a nicotine acetylcholine alpha 7 receptor agonist; a serotonin/norepinephrine reuptake inhibitor; moclobemide; brofaromine; befloxatone; toloxatone; gluoxetine; citalopram; excitalopram; fluvoxamine; sertraline; paroxetine; a dopamine D1 antagonist; zolmitriptan; divalproex; lithium; and guanfacine.

In yet further embodiments:
a. the subject is suffering from or susceptible to anxiety or anxiety disorder; and the second therapeutic agent is a SSRI or a SNRI.
b. the subject is suffering from or susceptible to Alzheimer's disease or dementia; and the second therapeutic agent is divalproex;
c. the subject is suffering from or susceptible to schizophrenia; and the second therapeutic agent is guanfacine; or
d. the subject is suffering from or susceptible to bipolar I disorder and the second therapeutic agent is selected from lithium and divalproex.

In certain embodiments, disclosed herein is a deuterium-enriched compound of Formula I or a pharmaceutically acceptable salt thereof:

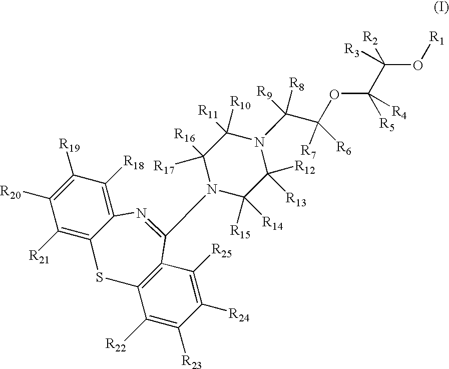

wherein R₁-R₂₅ are independently selected from H and D; and
the abundance of deuterium in R₁-R₂₅ is at least 4%.

In further embodiments, the abundance of deuterium in R₁-R₂₅ is selected from the group consisting of at least 4%, at least 6%, at least 14%, at least 19%, at least 26%, at least 32%, at least 39%, at least 45%, at least 52%, at least 58%, at least 65%, at least 71%, at least 77%, at least 84%, at least 90%, at least 97%, and 100%.

In yet further embodiments, the abundance of deuterium in R₁ is 100%.

In yet further embodiments, the abundance of deuterium in R₂-R₅ is selected from the group consisting of at least 25%, at least 50%, at least 75%, and 100%.

In yet further embodiments, the abundance of deuterium in R₆-R₉ is selected from the group consisting of at least 25%, at least 50%, at least 75%, and 100%.

In yet further embodiments, the abundance of deuterium in R₁₀-R₁₃ is selected from the group consisting of at least 25%, at least 50%, at least 75%, and 100%.

In yet further embodiments, the abundance of deuterium in R₁₄-R₁₇ is selected from the group consisting of at least 25%, at least 50%, at least 75%, and 100%.

In yet further embodiments, the abundance of deuterium in R₁₈-R₂₅ is selected from the group consisting of at least 13%, at least 25%, at least 38%, at least 50%, at least 63%, at least 75%, at least 88%, and 100%.

In yet further embodiments, the compound is selected from the group consisting of:

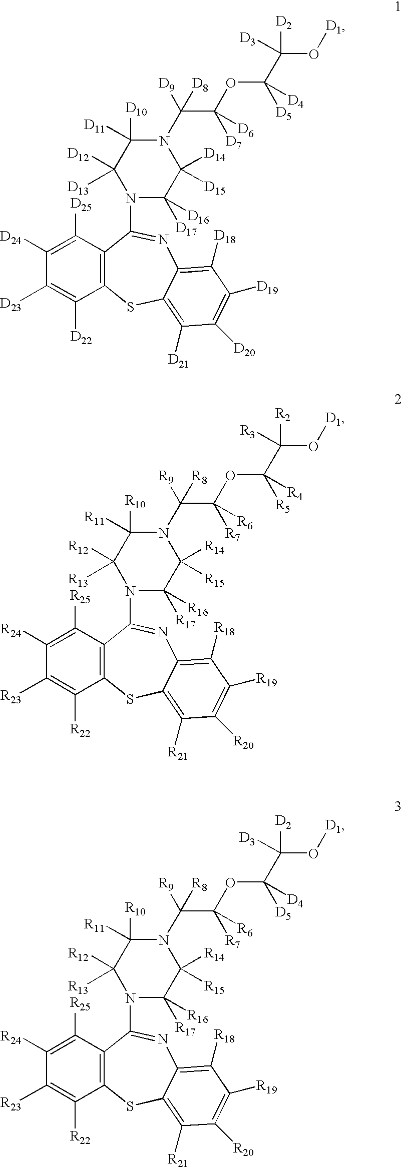

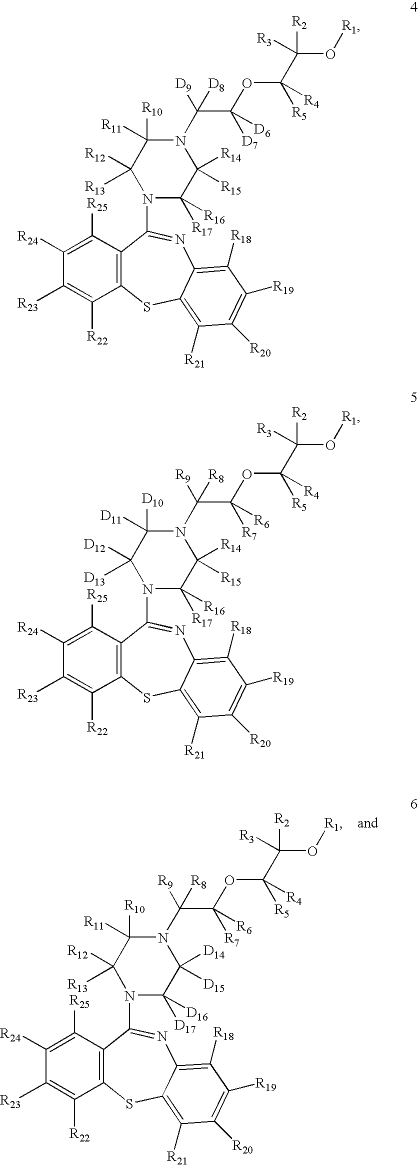

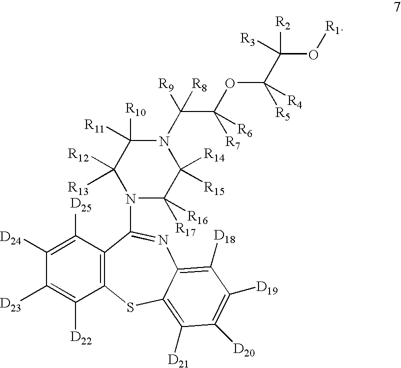

In yet further embodiments, the compound is selected from the group consisting of:

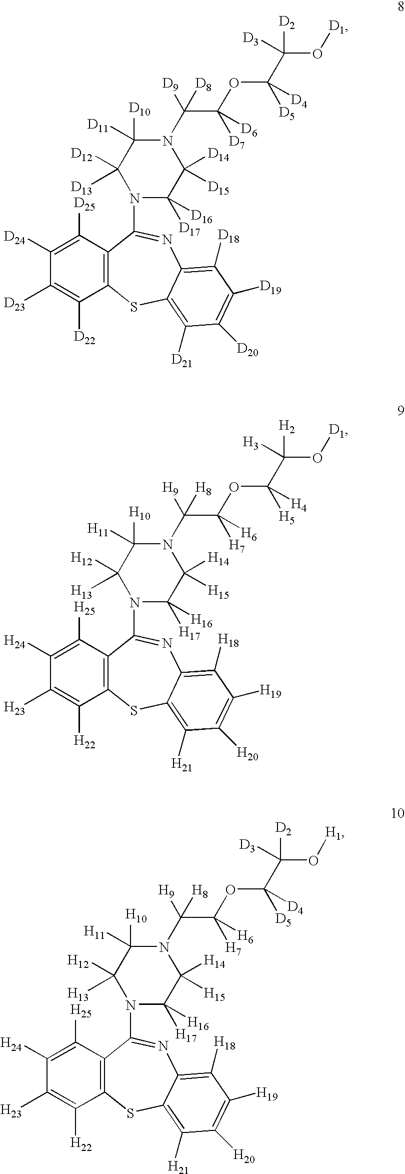

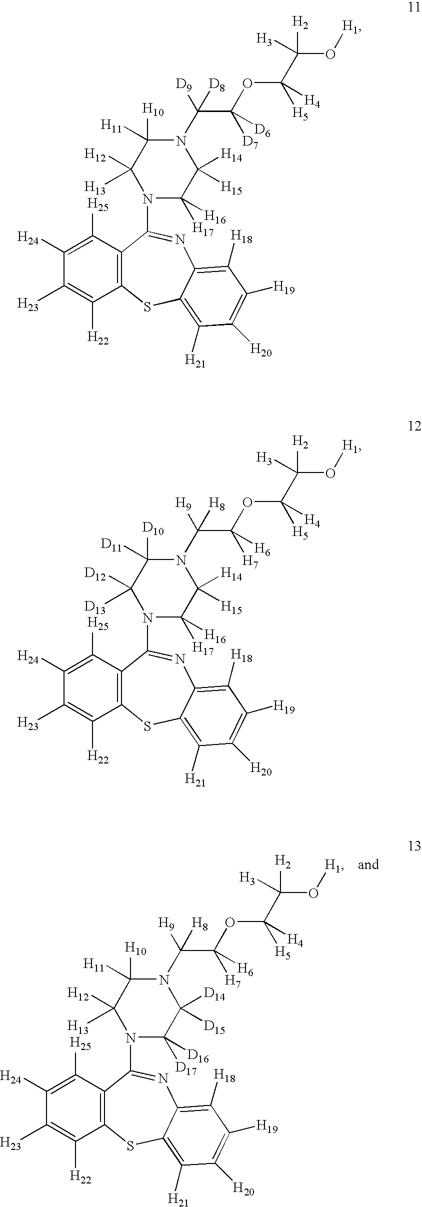

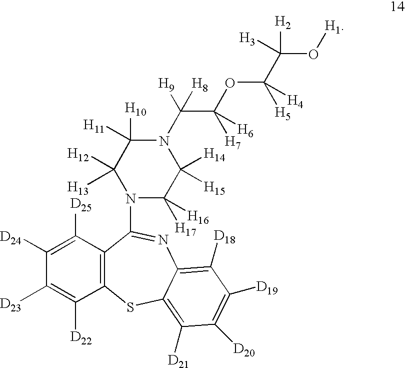

In certain embodiments, disclosed herein is an isolated deuterium-enriched compound of Formula I, or a pharmaceutically acceptable salt thereof:

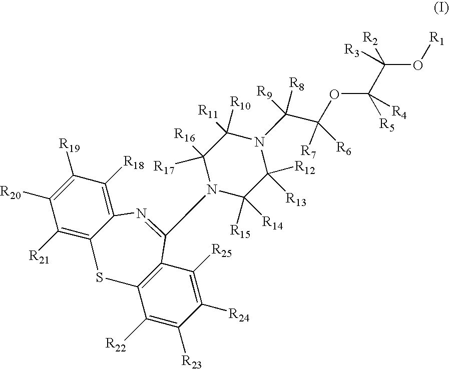

wherein R₁-R₂₅ are independently selected from H and D; and
the abundance of deuterium in R₁-R₂₅ is at least 4%.

In further embodiments, the abundance of deuterium in R₁-R₂₅ is selected from the group consisting of at least 4%, at least 6%, at least 14%, at least 19%, at least 26%, at least 32%, at least 39%, at least 45%, at least 52%, at least 58%, at least 65%, at least 71%, at least 77%, at least 84%, at least 90%, at least 97%, and 100%.

In yet further embodiments, the abundance of deuterium in R₁ is 100%.

In yet further embodiments, the compound is compound is selected from the group consisting of:

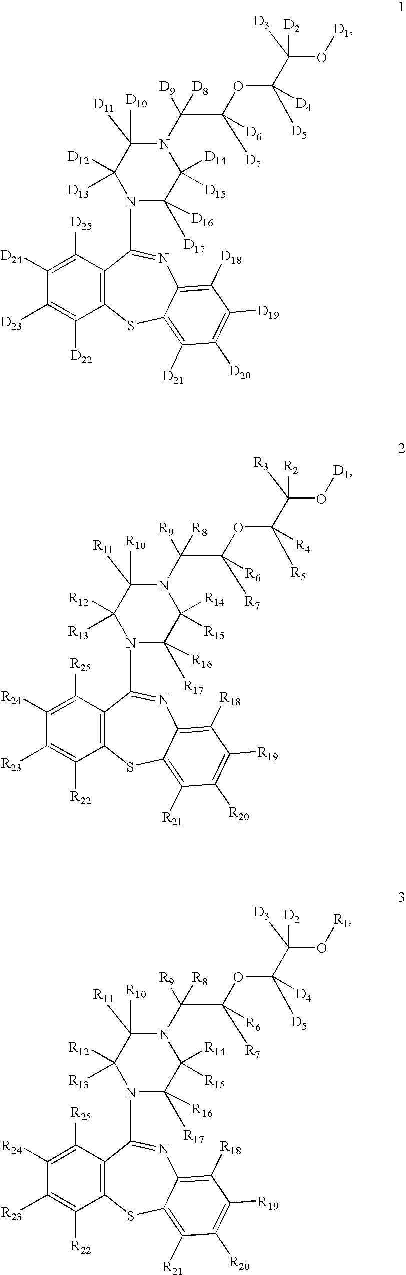

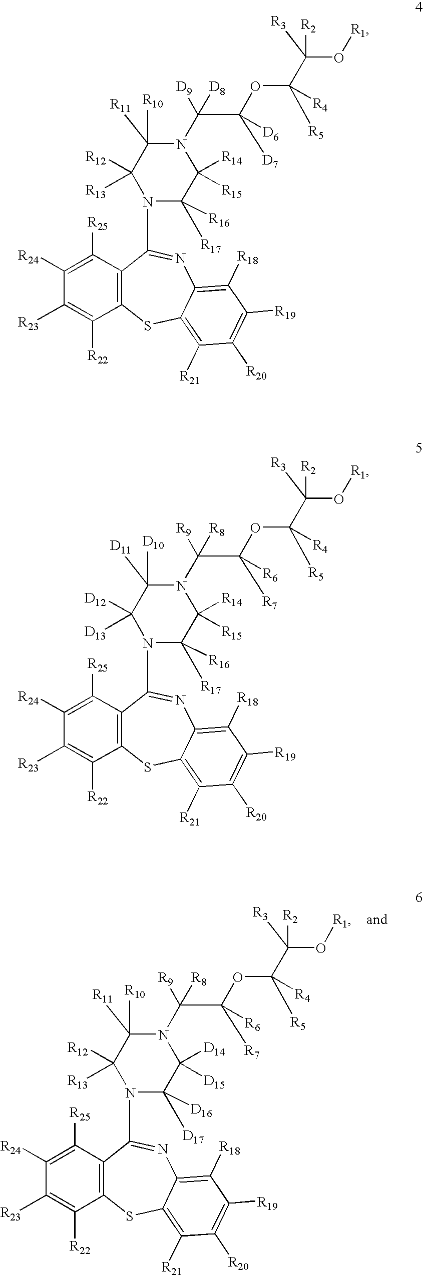

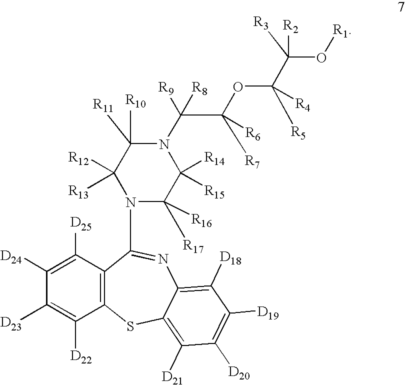

In yet further embodiments, the compound is compound is selected from the group consisting of:

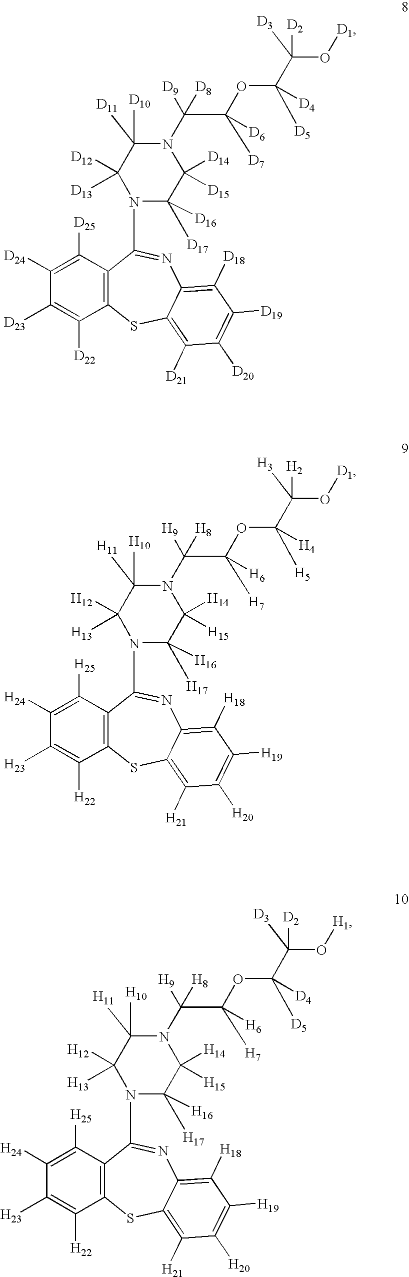

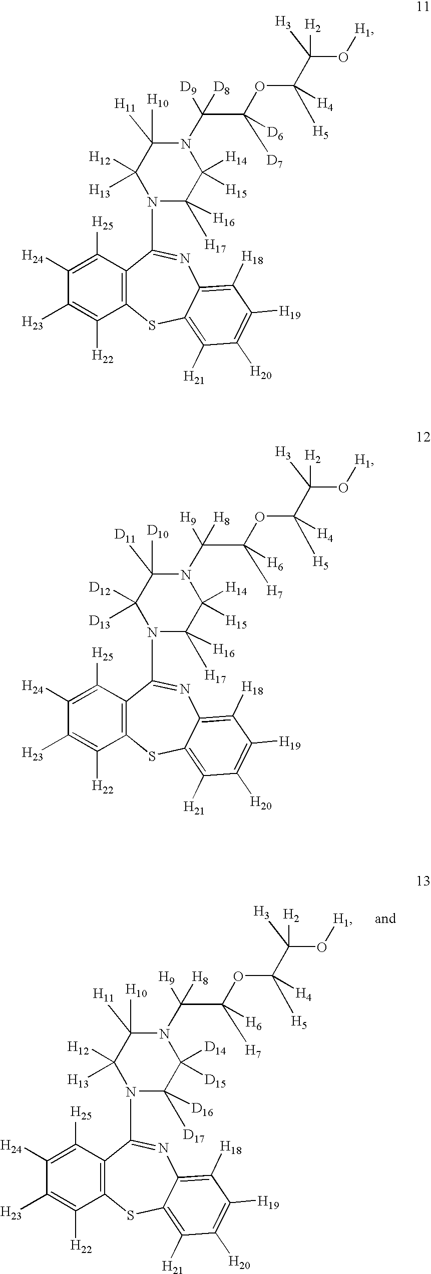

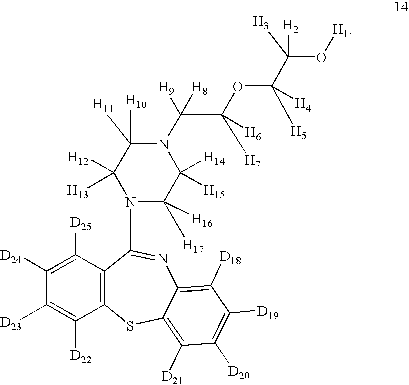

In certain embodiments, disclosed herein is a mixture of deuterium-enriched compounds of Formula I, or a pharmaceutically acceptable salt thereof:

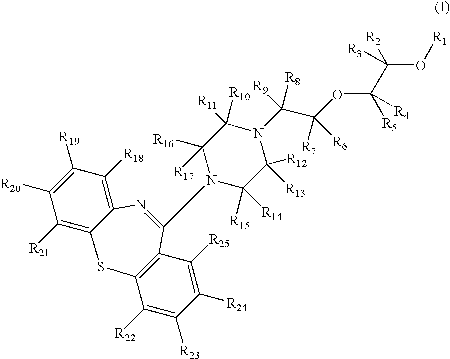

wherein R₁-R₂₅ are independently selected from H and D; and
the abundance of deuterium in R₁-R₂₅ is at least 4%.

In yet further embodiments, the compound is selected from the group consisting of:

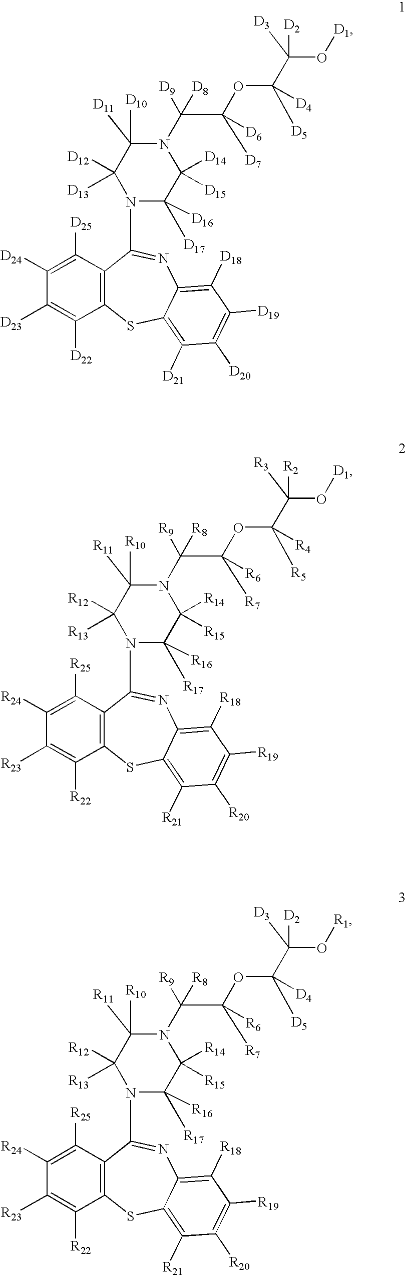

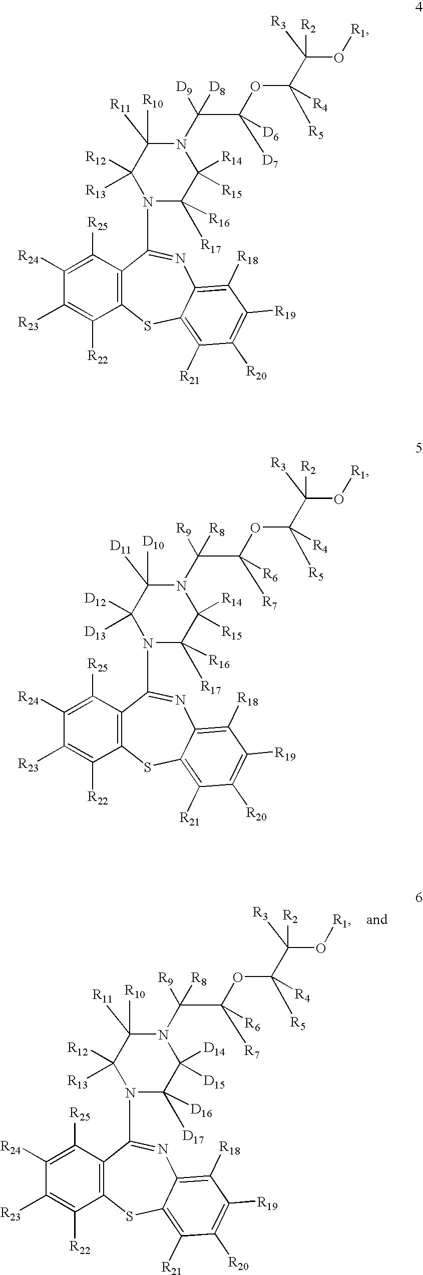

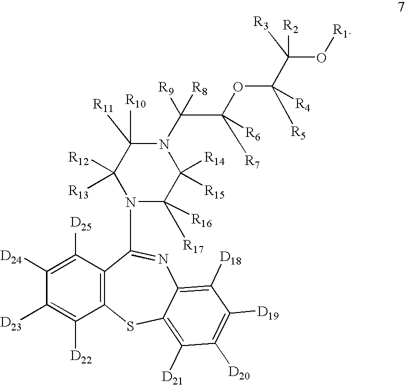

In yet further embodiments, the compound is selected from the group consisting of:

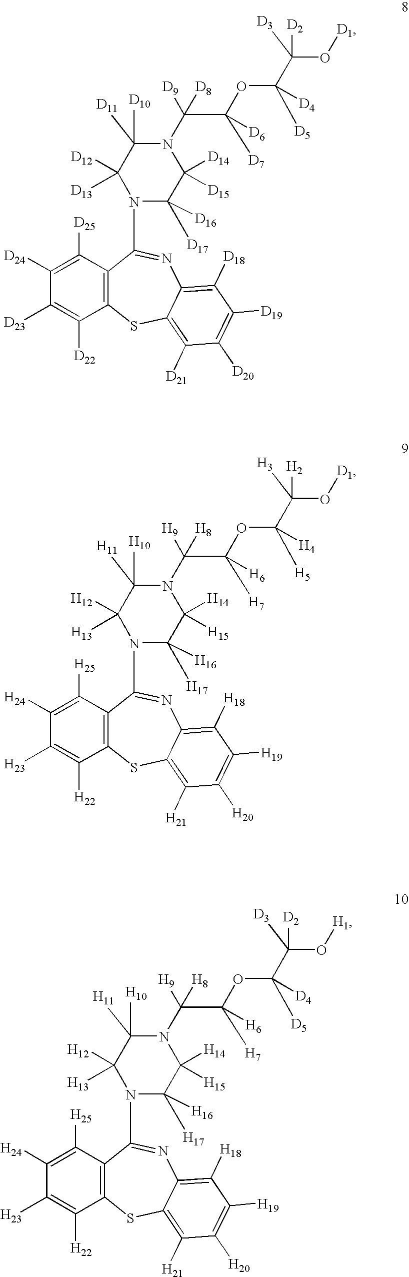

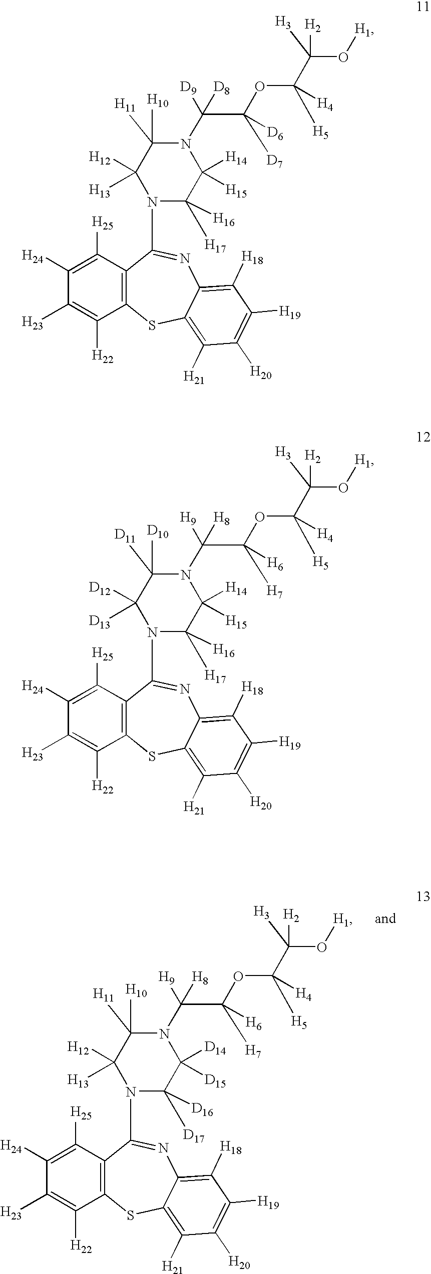

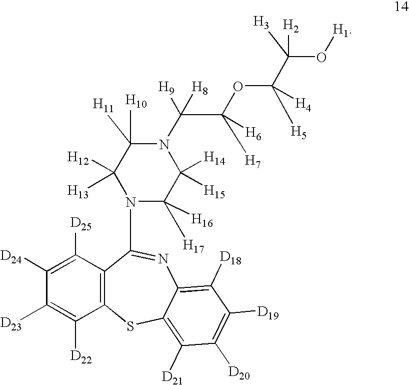

In certain embodiments, disclosed herein is a pharmaceutical composition, comprising: a pharmaceutically acceptable carrier and a therapeutically effective amount of a compound of Formula I, or a pharmaceutically acceptable salt form thereof.

In certain embodiments, disclosed herein is a method for treating a disease selected from schizophrenia and/or acute mania in bipolar disorder comprising: administering, to a patient in need thereof, a therapeutically effective amount of a compound of Formula I, or a pharmaceutically acceptable salt form thereof.

All publications and references cited herein are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

As used herein, the terms below have the meanings indicated.

The singular forms “a,” “an,” and “the” may refer to plural articles unless specifically stated otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

When ranges of values are disclosed, and the notation “from n₁ . . . to n₂” or “n₁-n₂” is used, where n₁ and n₂ are the numbers, then unless otherwise specified, this notation is intended to include the numbers themselves and the range between them. This range may be integral or continuous between and including the end values.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule such as R₁-R₂₅ or the symbol “D,” when used to represent a given position in a drawing of a molecular structure, means that the specified position is enriched with deuterium above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as D-isomers and L-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disorder” as used herein is intended to be generally synonymous, and is used interchangeably with, the terms “disease” and “condition” (as in medical condition), in that all reflect an abnormal condition of the human or animal body or of one of its parts that impairs normal functioning, is typically manifested by distinguishing signs and symptoms.

The terms “treat,” “treating,” and “treatment” are meant to include alleviating or abrogating a disorder or one or more of the symptoms associated with a disorder; or alleviating or eradicating the cause(s) of the disorder itself. As used herein, reference to “treatment” of a disorder is intended to include prevention. The terms “prevent,” “preventing,” and “prevention” refer to a method of delaying or precluding the onset of a disorder; and/or its attendant symptoms, barring a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “combination therapy” means the administration of two or more therapeutic agents to treat a therapeutic disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the disorders described herein.

The term “D1 receptor,” refers to a subclass of metabotropic G-protein-coupled receptors and/or transporters found extensively in the central nervous system, for which the neurotransmitter dopamine is the primary endogenous ligand. At least five different subtypes ( D1, D2, D3, D4, and D5) of dopamine receptors are known. The D1 subtype is the most abundant dopamine receptor in the central nervous system. Activation of D1 receptors is coupled to the G protein Gαs, which subsequently activates adenylyl cyclase, increasing the intracellular concentration of the second messenger cyclic adenosine monophosphate (cAMP). Increased cAMP in neurons is typically excitatory. Dysfunction of dopaminergic neurotransmission in the central nervous system has been implicated in a variety of neuropsychiatric disorders, including social phobia, Tourette's syndrome, Parkinson's disease, schizophrenia, neuroleptic malignant syndrome, attention-deficit hyperactivity disorder (ADHD), and drug and alcohol dependence.

The term “D2 receptor,” refers to a subclass of metabotropic G-protein-coupled receptors and/or transporters found extensively in the central nervous system, for which the neurotransmitter dopamine is the primary endogenous ligand. At least five different subtypes ( D1, D2, D3, D4, and D5) of dopamine receptors are known. D2 receptor activation is coupled to the G protein G_{αi}, which directly inhibits the formation of cAMP by inhibiting the enzyme adenylate cyclase. Decreased cAMP in neurons is typically inhibitory. Dysfunction of dopaminergic neurotransmission in the central nervous system has been implicated in a variety of neuropsychiatric disorders, including social phobia, Tourette's syndrome, Parkinson's disease, schizophrenia, neuroleptic malignant syndrome, attention-deficit hyperactivity disorder (ADHD), and drug and alcohol dependence. D2-receptor ligands may exhibit functional selectivity, i.e., modulation of D2 receptors by different ligands may activate different signal transduction pathways.

The term “alpha-1 adrenergic receptor,” refers to a member of the G protein-coupled receptor superfamily for which adrenaline and noradrenaline are the primary and secondary endogenous ligands. Upon activation, a heterotrimeric G protein, Gq, activates phospholipase C (PLC), which causes an increase in IP3 and calcium. The primary effect of alpha-1 adrenergic receptor agonism is vasoconstriction.

The term “alpha-2 adrenergic receptor,” refers to a member of the G protein-coupled receptor superfamily for which adrenaline and noradrenaline are the primary and secondary endogenous ligands. Upon activation, a G protein—Gi renders adenylate cyclase inactivated, resulting in decrease of cAMP. The primary effects of alpha-2 adrenergic receptor agonism is vasoconstriction and the mediation of synaptic transmission in pre- and post-synaptic nerve terminals, including decrease release of acetylcholine, decrease release of noradrenaline, and inhibition of the noradrenaline system in the brain.

The term “H1 receptor,” refers to a a class of G-protein coupled receptors with histamine as their endogenous ligand. There are four known histamine receptors: H1, H2, H3, and H4. Histamine H1 receptor are metabotropic G-protein-coupled receptors expressed throughout the body, specifically in smooth muscles, on vascular endothelial cells, in the heart, and in the central nervous system. The H1 receptor is linked to an intracellular G-protein (G_{q}) which activates phospholipase C and the phosphatidylinositol (PIP2) signalling pathway. The production of prostaglandin E2 synthase induces the release of histamine from neurons, consequentially causing systemic vasodilation, along with increased cell permeability due to its action on H1 receptors. Histamine H1 receptors are activated by endogenous histamine, which is released by neurons which have their cell bodies in the tuberomamillary nucleus of the hypothalamus. The histaminergic neurons of the tuberomammillary nucleus become active during the wake cycle. In the cortex, activation of H1 receptors leads to inhibition of cell membrane potassium channels. This depolarizes the neurons and increases the resistance of the neuronal cell membrane, bringing the cell closer to its firing threshold and increasing the excitatory voltage produced by a given excitatory current. H1 receptor antagonists typically produce drowsiness because they oppose this action, reducing neuronal excitation.

The term “5HT1A receptor,” refers to a subclass of a family of receptors for the neurotransmitter and peripheral signal mediator serotonin. The 5-HT1A receptor is coupled to an intracellular G-protein (Gᵢ/Gₒ) which inhibits the formation of cAMP by inhibiting the enzyme adenylate cyclase. 5-HT1A acts on the central nervous system, where it induces neuronal inhibition and regulates various behaviours, including sleep, feeding, thermoregulation, aggression, and anxiety.

The term “5HT2 receptor,” refers to a subclass of a family of receptors for the neurotransmitter and peripheral signal mediator serotonin. 5-HT2 receptors mediate many of the central and peripheral physiologic functions of serotonin. Cardiovascular effects include vasoconstriction and platelet aggregation. Central nervous system effects include neuronal sensitization to tactile stimuli and mediation of hallucinogenic effects of phenylisopropylamine hallucinogens. Members of the 5-HT2 subclass include 5-HT2A, 5-HT2B, and 5-HT2C. The 5-HT2A receptor is known primarily to couple to the G_{αq} signal transduction pathway. Upon receptor stimulation with agonist, G_{αq} and beta-gamma subunits dissociate to initiate downstream effector pathways. G_{αq} stimulates phospholipase C (PLC) activity, which subsequently promotes the release of diacylglycerol (DAG) and inositol triphosphate (IP3), which in turn stimulate protein kinase C (PKC) activity and Ca²⁺ release. Effects of 5-HT2A activation in the central nervous system include neuronal excitation, which mediates a number of behavioural effects, including anxiety. The 5-HT2B receptor subtype mediates presynaptic inhibition in the central nervous system as well as vasoconstriction. 5-HT2C receptors play an important role in the modulation of monoaminergic transmission, mood, motor behaviour, appetite and endocrine secretion, and alterations in their functional status have been detected in anxiodepressive states. 5-HT2-receptor ligands may exhibit functional selectivity, i.e., modulation of 5-HT2 receptors by different ligands may activate different signal transduction pathways.

The term “D1 receptor-mediated disorder,” refers to a disorder that is characterized by abnormal D1 receptor activity. A D1 receptor-mediated disorder may be completely or partially mediated by modulating D1 receptors. In particular, a D1 receptor-mediated disorder is one in which modulation of D1 receptors results in some effect on the underlying disorder e.g., administration of a D1 receptor modulator results in some improvement in at least some of the patients being treated.

The term “D2 receptor-mediated disorder,” refers to a disorder that is characterized by abnormal D2 receptor activity. A D2 receptor-mediated disorder may be completely or partially mediated by modulating D2 receptors. In particular, a D2 receptor-mediated disorder is one in which modulation of D2 receptors results in some effect on the underlying disorder e.g., administration of a D2 receptor modulator results in some improvement in at least some of the patients being treated.

The term “alpha-1 adrenergic receptor-mediated disorder,” refers to a disorder that is characterized by abnormal alpha-1 adrenergic receptor activity. An alpha-1 adrenergic receptor-mediated disorder may be completely or partially mediated by modulating alpha-1 adrenergic receptors. In particular, an alpha-1 adrenergic receptor-mediated disorder is one in which modulation of alpha-1 adrenergic receptors results in some effect on the underlying disorder e.g., administration of an alpha-1 adrenergic receptor modulator results in some improvement in at least some of the patients being treated.

The term “alpha-2 adrenergic receptor-mediated disorder,” refers to a disorder that is characterized by abnormal alpha-2 adrenergic receptor activity. An alpha-2 adrenergic receptor-mediated disorder may be completely or partially mediated by modulating alpha-2 adrenergic receptors. In particular, an alpha-2 adrenergic receptor-mediated disorder is one in which modulation of alpha-2 adrenergic receptors results in some effect on the underlying disorder e.g., administration of an alpha-2 adrenergic receptor modulator results in some improvement in at least some of the patients being treated.

The term “H1 receptor-mediated disorder,” refers to a disorder that is characterized by abnormal H1 receptor activity. A H1 receptor-mediated disorder may be completely or partially mediated by modulating H1 receptors. In particular, a H1 receptor-mediated disorder is one in which modulation of H1 receptors results in some effect on the underlying disorder e.g., administration of a H1 receptor modulator results in some improvement in at least some of the patients being treated.

The term “5-HT1A receptor-mediated disorder,” refers to a disorder that is characterized by abnormal 5-HT1A receptor activity. A 5-HT1A receptor-mediated disorder may be completely or partially mediated by modulating 5-HT1A receptors. In particular, a 5-HT1A receptor-mediated disorder is one in which modulation of 5-HT1A receptors results in some effect on the underlying disorder e.g., administration of a 5-HT1A receptor modulator results in some improvement in at least some of the patients being treated.

The term “5-HT2 receptor-mediated disorder,” refers to a disorder that is characterized by abnormal 5-HT2 receptor activity. A 5-HT2 receptor-mediated disorder may be completely or partially mediated by modulating 5-HT2 receptors. In particular, a 5-HT2 receptor-mediated disorder is one in which modulation of 5-HT2 receptors results in some effect on the underlying disorder e.g., administration of a 5-HT2 receptor modulator results in some improvement in at least some of the patients being treated.

The term “D1 receptor modulator,” refers to the ability of a compound disclosed herein to alter the function of D1 receptors. A D1 receptor modulator may activate the activity of a D1 receptor, may activate or inhibit the activity of a D1 receptor depending on the concentration of the compound exposed to the D1 receptor, or may inhibit the activity of a D1 receptor. Such activation or inhibition may be contingent on the occurrence of a specific event, such as activation of a signal transduction pathway, and/or may be manifest only in particular cell types. The term “modulate D1 receptor” or “D1 receptor modulation” also refers to altering the function of a D1 receptor by increasing or decreasing the probability that a complex forms between a D1 receptor and a natural binding partner. A D1 receptor modulator may increase the probability that such a complex forms between the D1 receptor and the natural binding partner, may increase or decrease the probability that a complex forms between the D1 receptor and the natural binding partner depending on the concentration of the compound exposed to the D1 receptor, and or may decrease the probability that a complex forms between the D1 receptor and the natural binding partner.

The term “D2 receptor modulator,” refers to the ability of a compound disclosed herein to alter the function of D2 receptors. A D2 receptor modulator may activate the activity of a D2 receptor, may activate or inhibit the activity of a D2 receptor depending on the concentration of the compound exposed to the D1 receptor, or may inhibit the activity of a D2 receptor. Such activation or inhibition may be contingent on the occurrence of a specific event, such as activation of a signal transduction pathway, and/or may be manifest only in particular cell types. The term “modulate D2 receptor” or “D2 receptor modulation” also refers to altering the function of a D2 receptor by increasing or decreasing the probability that a complex forms between a D2 receptor and a natural binding partner. A D2 receptor modulator may increase the probability that such a complex forms between the D2 receptor and the natural binding partner, may increase or decrease the probability that a complex forms between the D2 receptor and the natural binding partner depending on the concentration of the compound exposed to the D2 receptor, and or may decrease the probability that a complex forms between the D2 receptor and the natural binding partner.

The term “alpha-1 adrenergic receptor modulator,” refers to the ability of a compound disclosed herein to alter the function of alpha-1 adrenergic receptors. An alpha-1 adrenergic receptor modulator may activate the activity of an alpha-1 adrenergic receptor, may activate or inhibit the activity of an alpha-1 adrenergic receptor depending on the concentration of the compound exposed to the alpha-1 adrenergic receptor, or may inhibit the activity of an alpha-1 adrenergic receptor. Such activation or inhibition may be contingent on the occurrence of a specific event, such as activation of a signal transduction pathway, and/or may be manifest only in particular cell types. The term “modulate alpha-1 adrenergic receptor” or “alpha-1 adrenergic receptor modulation” also refers to altering the function of an alpha-1 adrenergic receptor by increasing or decreasing the probability that a complex forms between an alpha-1 adrenergic receptor and a natural binding partner. An alpha-1 adrenergic receptor modulator may increase the probability that such a complex forms between the alpha-1 adrenergic receptor and the natural binding partner, may increase or decrease the probability that a complex forms between the alpha-1 adrenergic receptor and the natural binding partner depending on the concentration of the compound exposed to the alpha-1 adrenergic receptor, and or may decrease the probability that a complex forms between the alpha-1 adrenergic receptor and the natural binding partner.

The term “alpha-2 adrenergic receptor modulator,” refers to the ability of a compound disclosed herein to alter the function of alpha-2 adrenergic receptors. An alpha-2 adrenergic receptor modulator may activate the activity of an alpha-2 adrenergic receptor, may activate or inhibit the activity of an alpha-2 adrenergic receptor depending on the concentration of the compound exposed to the alpha-2 adrenergic receptor, or may inhibit the activity of an alpha-2 adrenergic receptor. Such activation or inhibition may be contingent on the occurrence of a specific event, such as activation of a signal transduction pathway, and/or may be manifest only in particular cell types. The term “modulate alpha-2 adrenergic receptor” or “alpha-2 adrenergic receptor modulation” also refers to altering the function of an alpha-2 adrenergic receptor by increasing or decreasing the probability that a complex forms between an alpha-2 adrenergic receptor and a natural binding partner. An alpha-2 adrenergic receptor modulator may increase the probability that such a complex forms between the alpha-2 adrenergic receptor and the natural binding partner, may increase or decrease the probability that a complex forms between the alpha-2 adrenergic receptor and the natural binding partner depending on the concentration of the compound exposed to the alpha-2 adrenergic receptor, and or may decrease the probability that a complex forms between the alpha-2 adrenergic receptor and the natural binding partner.

The term “H1 receptor modulator,” refers to the ability of a compound disclosed herein to alter the function of H1 receptors. A H1 receptor modulator may activate the activity of a H1 receptor, may activate or inhibit the activity of a H1 receptor depending on the concentration of the compound exposed to the H1 receptor, or may inhibit the activity of a H1 receptor. Such activation or inhibition may be contingent on the occurrence of a specific event, such as activation of a signal transduction pathway, and/or may be manifest only in particular cell types. The term “modulate H1 receptor” or “H1 receptor modulation” also refers to altering the function of a H1 receptor by increasing or decreasing the probability that a complex forms between a H1 receptor and a natural binding partner. A H1 receptor modulator may increase the probability that such a complex forms between the H1 receptor and the natural binding partner, may increase or decrease the probability that a complex forms between the H1 receptor and the natural binding partner depending on the concentration of the compound exposed to the H1 receptor, and or may decrease the probability that a complex forms between the H1 receptor and the natural binding partner.

The term “5-HT1A receptor modulator,” refers to the ability of a compound disclosed herein to alter the function of 5-HT1A receptors. A 5-HT1A receptor modulator may activate the activity of a 5-HT1A receptor, may activate or inhibit the activity of a 5-HT1A receptor depending on the concentration of the compound exposed to the 5-HT1A receptor, or may inhibit the activity of a 5-HT1A receptor. Such activation or inhibition may be contingent on the occurrence of a specific event, such as activation of a signal transduction pathway, and/or may be manifest only in particular cell types. The term “modulate 5-HT1A receptor” or “5-HT1A receptor modulation” also refers to altering the function of a 5-HT1A receptor by increasing or decreasing the probability that a complex forms between a 5-HT1A receptor and a natural binding partner. A 5-HT1A receptor modulator may increase the probability that such a complex forms between the 5-HT1A receptor and the natural binding partner, may increase or decrease the probability that a complex forms between the 5-HT1A receptor and the natural binding partner depending on the concentration of the compound exposed to the 5-HT1A receptor, and or may decrease the probability that a complex forms between the 5-HT1A receptor and the natural binding partner.

The term “5-HT2 receptor modulator,” refers to the ability of a compound disclosed herein to alter the function of 5-HT2 receptors. A 5-HT2 receptor modulator may activate the activity of a 5-HT2 receptor, may activate or inhibit the activity of a 5-HT2 receptor depending on the concentration of the compound exposed to the 5-HT2 receptor, or may inhibit the activity of a 5-HT2 receptor. Such activation or inhibition may be contingent on the occurrence of a specific event, such as activation of a signal transduction pathway, and/or may be manifest only in particular cell types. The term “modulate 5-HT2 receptor” or “5-HT2 receptor modulation” also refers to altering the function of a 5-HT2 receptor by increasing or decreasing the probability that a complex forms between a 5-HT2 receptor and a natural binding partner. A 5-HT2 receptor modulator may increase the probability that such a complex forms between the 5-HT2 receptor and the natural binding partner, may increase or decrease the probability that a complex forms between the 5-HT2 receptor and the natural binding partner depending on the concentration of the compound exposed to the 5-HT2 receptor, and or may decrease the probability that a complex forms between the 5-HT2 receptor and the natural binding partner.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenecity, are commensurate with a reasonable benefit/risk ratio, and are effective for their intended use.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The terms “active ingredient,” “active compound,” and “active substance” refer to a compound, which is administered, alone or in combination with one or more pharmaceutically acceptable excipients or carriers, to a subject for treating, preventing, or ameliorating one or more symptoms of a disorder.

The terms “drug,” “therapeutic agent,” and “chemotherapeutic agent” refer to a compound, or a pharmaceutical composition thereof, which is administered to a subject for treating, preventing, or ameliorating one or more symptoms of a disorder.

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function do not include modifying the duration or place of release of the active substance from a dosage form as compared with a conventional immediate release dosage form.

The term “prodrug” refers to a compound functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The compounds disclosed herein can exist as therapeutically acceptable salts. The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts. For a more complete discussion of the preparation and selection of salts, refer to “Handbook of Pharmaceutical Salts, Properties, and Use,” Stah and Wermuth, Ed.; (Wiley-VCH and VHCA, Zurich, 2002) and Berge et al., J. Pharm. Sci. 1977, 66, 1-19.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

While it may be possible for the compounds of the subject invention to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The compositions include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Disclosed herein are methods of treating a D1 receptor-mediated disorder, a D2 receptor-mediated disorder, an alpha-1 adrenergic receptor-mediated disorder, an alpha-2 adrenergic receptor-mediated disorder, a H1 receptor-mediated disorder, a 5-HT1A receptor-mediated disorder, and/or a 5-HT2 receptor-mediated disorder comprising administering to a subject having or suspected to have such a disorder, a therapeutically effective amount of a compound as disclosed herein or a pharmaceutically acceptable salt, solvate, or prodrug thereof.

D1 receptor-mediated disorders, D2 receptor-mediated disorders, alpha-1 adrenergic receptor-mediated disorders, alpha-2 adrenergic receptor-mediated disorders, H1 receptor-mediated disorders, 5-HT1A receptor-mediated disorders, and/or 5-HT2 receptor-mediated disorders, include, but are not limited to, schizophrenia, schizoaffective disorders, mania (manic disorder), bipolar I disorder, bipolar II disorder, depression associated with bipolar disorders, unipolar depression, Alzheimer's disease, dementia, Parkinson's disease, alcoholism, substance-related disorders, generalized agitation, generalized anxiety, anxiety disorders, anxiety neuroses, major depression (major depressive disorder), borderline personality disorder, post-traumatic stress disorder, primary insomnia, anorexia nervosa, social phobia, manic-depressive psychoses, mood disorders, psychotic disorders, psychosis, fibromyalgia, Tourette's syndrome and obsessive-compulsive disorder, and/or any disorder which can lessened, alleviated, or prevented by administering a D1 receptor, D2 receptor, alpha-1 adrenergic receptor, alpha-2 adrenergic receptor, H1 receptor, 5-HT1A receptor, and/or 5-HT2 receptor modulator.

In certain embodiments, a method of treating a D1 receptor-mediated disorder, a D2 receptor-mediated disorder, an alpha-1 adrenergic receptor-mediated disorder, an alpha-2 adrenergic receptor-mediated disorder, a H1 receptor-mediated disorder, a 5-HT1A receptor-mediated disorder, and/or a 5-HT2 receptor-mediated disorder comprises administering to the subject a therapeutically effective amount of a compound of as disclosed herein, or a pharmaceutically acceptable salt, solvate, or prodrug thereof, so as to affect: (1) decreased inter-individual variation in plasma levels of the compound or a metabolite thereof; (2) increased average plasma levels of the compound or decreased average plasma levels of at least one metabolite of the compound per dosage unit; (3) decreased inhibition of, and/or metabolism by at least one cytochrome P₄₅₀ or monoamine oxidase isoform in the subject; (4) decreased metabolism via at least one polymorphically-expressed cytochrome P₄₅₀ isoform in the subject; (5) at least one statistically-significantly improved disorder-control and/or disorder-eradication endpoint; (6) an improved clinical effect during the treatment of the disorder, (7) prevention of recurrence, or delay of decline or appearance, of abnormal alimentary or hepatic parameters as the primary clinical benefit, or (8) reduction or elimination of deleterious changes in any diagnostic hepatobiliary function endpoints, as compared to the corresponding non-isotopically enriched compound.

In certain embodiments, inter-individual variation in plasma levels of the compounds as disclosed herein, or metabolites thereof, is decreased; average plasma levels of the compound as disclosed herein are increased; average plasma levels of a metabolite of the compound as disclosed herein are decreased; inhibition of a cytochrome P₄₅₀ or monoamine oxidase isoform by a compound as disclosed herein is decreased; or metabolism of the compound as disclosed herein by at least one polymorphically-expressed cytochrome P₄₅₀ isoform is decreased; by greater than about 5%, greater than about 10%, greater than about 20%, greater than about 30%, greater than about 40%, or by greater than about 50% as compared to the corresponding non-isotopically enriched compound.

Plasma levels of the compound as disclosed herein, or metabolites thereof, may be measured using the methods described by Li et al. Rapid Communications in Mass Spectrometry 2005, 19, 1943-1950, Wen et al., Zhongguo Yaofang 2007, 18(32), 2504-2506, Barret et al., Journal of Pharmaceutical and Biomedical Analysis 2007, 44(2), 498-505, Belal et al., Journal of Liquid Chromatography & Related Technologies 2008, 31(9), 1283-1298, and references cited therein.

Examples of cytochrome P₄₅₀ isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

Examples of monoamine oxidase isoforms in a mammalian subject include, but are not limited to, MAO_{A}, and MAO_{B}.

The inhibition of the cytochrome P₄₅₀ isoform is measured by the method of Ko et al. (British Journal of Clinical Pharmacology, 2000, 49, 343-351). The inhibition of the MAO_{A} isoform is measured by the method of Weyler et al. (J. Biol Chem. 1985, 260, 13199-13207). The inhibition of the MAO_{B} isoform is measured by the method of Uebelhack et al. (Pharmacopsychiatry, 1998, 31, 187-192).

Examples of polymorphically-expressed cytochrome P₄₅₀ isoforms in a mammalian subject include, but are not limited to, CYP2C8, CYP2C9, CYP2C19, and CYP2D6.

The metabolic activities of liver microsomes, cytochrome P₄₅₀ isoforms, and monoamine oxidase isoforms are measured by the methods described herein.

Examples of improved disorder-control and/or disorder-eradication endpoints, or improved clinical effects include, but are not limited to, improved Clinical Global Impression Improvement (CGI-I) scores, improved Montgomery-Asberg Depression Rating Scale (MADRS) total scores, improved assessment of positive and negative syndromes on the scale-excitatory subscale, increased time to occurrence of a mood event, increased time to the discontinuation of treatment for any cause, reduced proportion of heavy drinking days from baseline to week 12, and improved young mania rating scale (YMRS) scores. Dando et al., Drugs 65(17) 2533-51 (2005); Dev et al., Drug Safety 23(4) 295-307 (2000); Keating et al., Drugs 67(7) 1077-95 (2007); Khouzam et al., Expert Rev. Neurotherapeautics 6(2) 2006); and Drug Report for Quetiapine, Thompson Investigational Drug Database (Aug. 12, 2008).

Examples of diagnostic hepatobiliary function endpoints include, but are not limited to, alanine aminotransferase (“ALT”), serum glutamic-pyruvic transaminase (“SGPT”), aspartate aminotransferase (“AST” or “SGOT”), ALT/AST ratios, serum aldolase, alkaline phosphatase (“ALP”), ammonia levels, bilirubin, gamma-glutamyl transpeptidase (“GGTP,” “γ-GTP,” or “GGT”), leucine aminopeptidase (“LAP”), liver biopsy, liver ultrasonography, liver nuclear scan, 5′-nucleotidase, and blood protein. Hepatobiliary endpoints are compared to the stated normal levels as given in “Diagnostic and Laboratory Test Reference”, 4^{th} edition, Mosby, 1999. These assays are run by accredited laboratories according to standard protocol.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Combination Therapy

The compounds disclosed herein may also be combined or used in combination with other agents useful in the treatment of D1 receptor-mediated disorders, D2 receptor-mediated disorders, alpha-1 adrenergic receptor-mediated disorders, alpha-2 adrenergic receptor-mediated disorders, H1 receptor-mediated disorders, 5-HT1A receptor-mediated disorders, and/or 5-HT2 receptor-mediated disorders. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

In certain embodiments, the compounds disclosed herein can be combined with one or more antidepressants and antipsychotics.

In further embodiments, the compounds disclosed herein can be combined with an antidepressant selected from the group consisting of citalopram, escitalopram, paroxetine, fluotexine, fluvoxamine, sertraline, isocarboxazid, moclobemide, phenelzine, tranylcypromine, amitriptyline, clomipramine, desipramine, dosulepin, imipramine, nortriptyline, protriptyline, trimipramine, lofepramine, maprotiline, amoxapine, mianserin, mirtazapine, duloxetine, nefazodone, reboxetine, trazodone, venlafaxine, tianeptine, and milnacipran.

In further embodiments, the compounds disclosed herein can be combined with an antipsychotic selected from the group consisting of haloperidol, chlorpromazine, fluphenazine, perphenazine, prochlorperazine, thioridazine, trifluoperazine, mesoridazine, promazine, triflupromazine, levomepromazine, promethazine, chlorprothixene, flupenthixol, thiothixene, zuclopenthixol, clozapine, olanzapine, quetiapine, ziprasidone, risperidone, amisulpride, paliperidone, bifeprunox, norclozapine, aripiprazole, tetrabenazine, and cannabidiol.

In further embodiments, the compounds disclosed herein can be combined with lithium or valproate.

The compounds disclosed herein can also be administered in combination with other classes of compounds, including, but not limited to, norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

Thus, in another aspect, certain embodiments provide methods for treating D1 receptor-mediated disorders, D2 receptor-mediated disorders, alpha-1 adrenergic receptor-mediated disorders, alpha-2 adrenergic receptor-mediated disorders, H1 receptor-mediated disorders, 5-HT1A receptor-mediated disorders, and/or 5-HT2 receptor-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of D1 receptor-mediated disorders, D2 receptor-mediated disorders, alpha-1 adrenergic receptor-mediated disorders, alpha-2 adrenergic receptor-mediated disorders, H1 receptor-mediated-mediated disorders, 5-HT1A receptor-mediated disorders, and/or 5-HT2 receptor-mediated disorders.

### General Synthetic Methods for Preparing Compounds

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof, and/or procedures found in WO 2007004234; WO 2005012274; WO 2006027789; WO 2006001619; WO 2006117700; WO 2005028459; WO 2005028458; WO 2005028457; WO 2004076431; WO 2001055125; US 20070293471; US 20060063927; US 20050080072; EP 1602650; EP 282236; and EP 240228, which are hereby incorporated in their entirety, and references cited therein and routine modifications thereof. Compounds as disclosed herein can also be prepared as shown in any of the following schemes and routine modifications thereof.

The following schemes can be used to practice the present invention. Any position shown as hydrogen can be optionally substituted with deuterium.

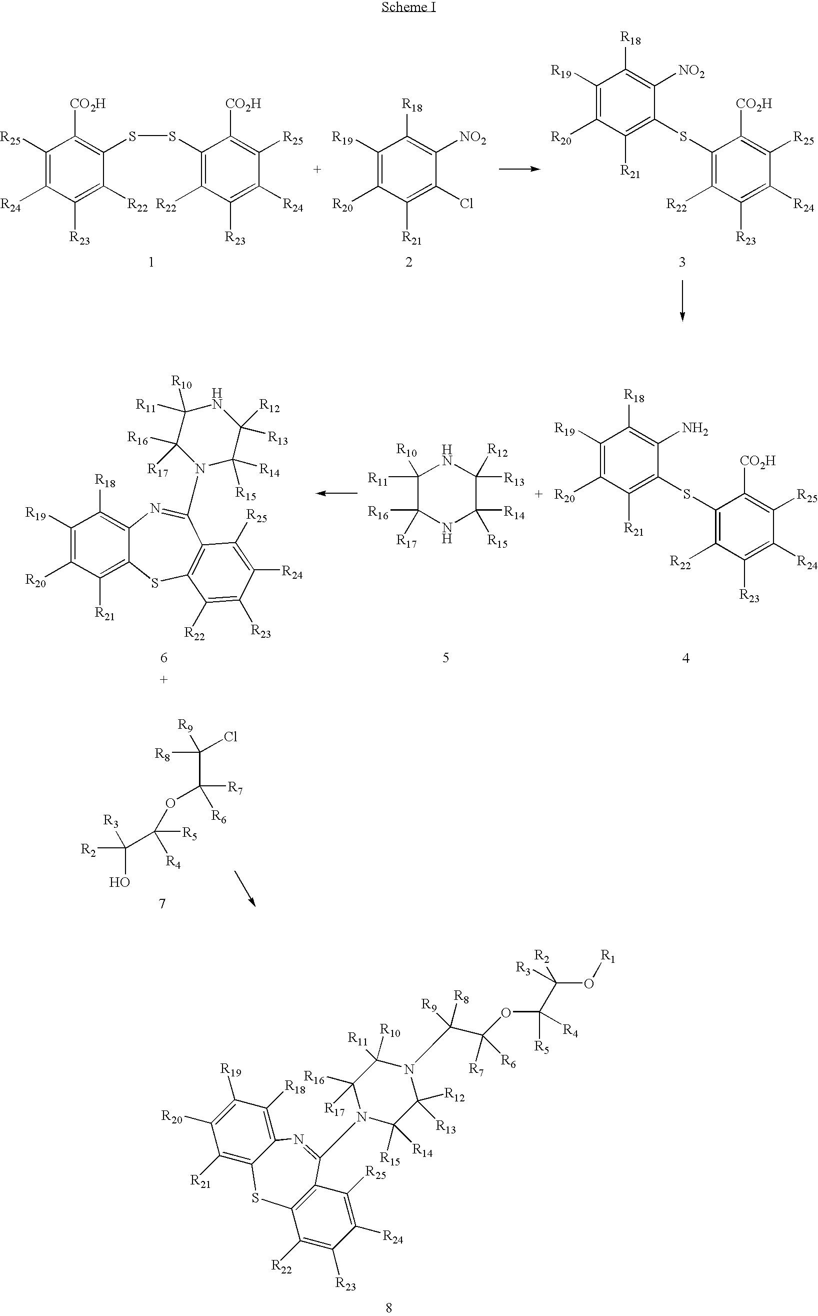

Compound 1 is treated with an appropriate reducing agent, such as zinc metal, in the presence of an appropriate base, such as sodium hydroxide, and reacted with compound 2, in an appropriate solvent, such as water, to give compound 3. Compound 3 is treated with an appropriate reducing agent, such as Raney-nickel/hydrogen, in an appropriate solvent, such as water, to give compound 4. Compound 4 is treated with an appropriate chlorinating agent, such as phosphorous oxychloride, in the presence of an appropriate base, such as dimethyl aniline, followed by compound 5, in an appropriate solvent, such as toluene, to give compound 6. Compound 6 is reacted with compound 7 in the presence of an appropriate base, such as sodium carbonate, and an appropriate catalyst, such as sodium iodide, in an appropriate solvent, such as a mixture of n-propyl alcohol and N-methyl pyrrolidone, to give a compound 8 of Formula I.

Deuterium can be incorporated to different positions synthetically, according to the synthetic procedures as shown in Scheme I, by using appropriate deuterated intermediates. For example, to introduce deuterium at one or more positions of R₂₂-R₂₅, compound 1 with the corresponding deuterium substitutions can be used. To introduce deuterium at one or more positions of R₁₈-R₂₁, compound 2 with the corresponding deuterium substitutions can be used. To introduce deuterium at one or more positions of R₁₀-R₁₇, compound 5 with the corresponding deuterium substitutions can be used. To introduce deuterium at one or more positions of R₁-R₉, compound 7 with the corresponding deuterium substitutions can be used.

Deuterium can be incorporated to various positions having an exchangeable proton, such as the hydroxyl O—H, via proton-deuterium equilibrium exchange. For example, to introduce deuterium at R₁, this proton may be replaced with deuterium selectively or non-selectively through a proton-deuterium exchange method known in the art.

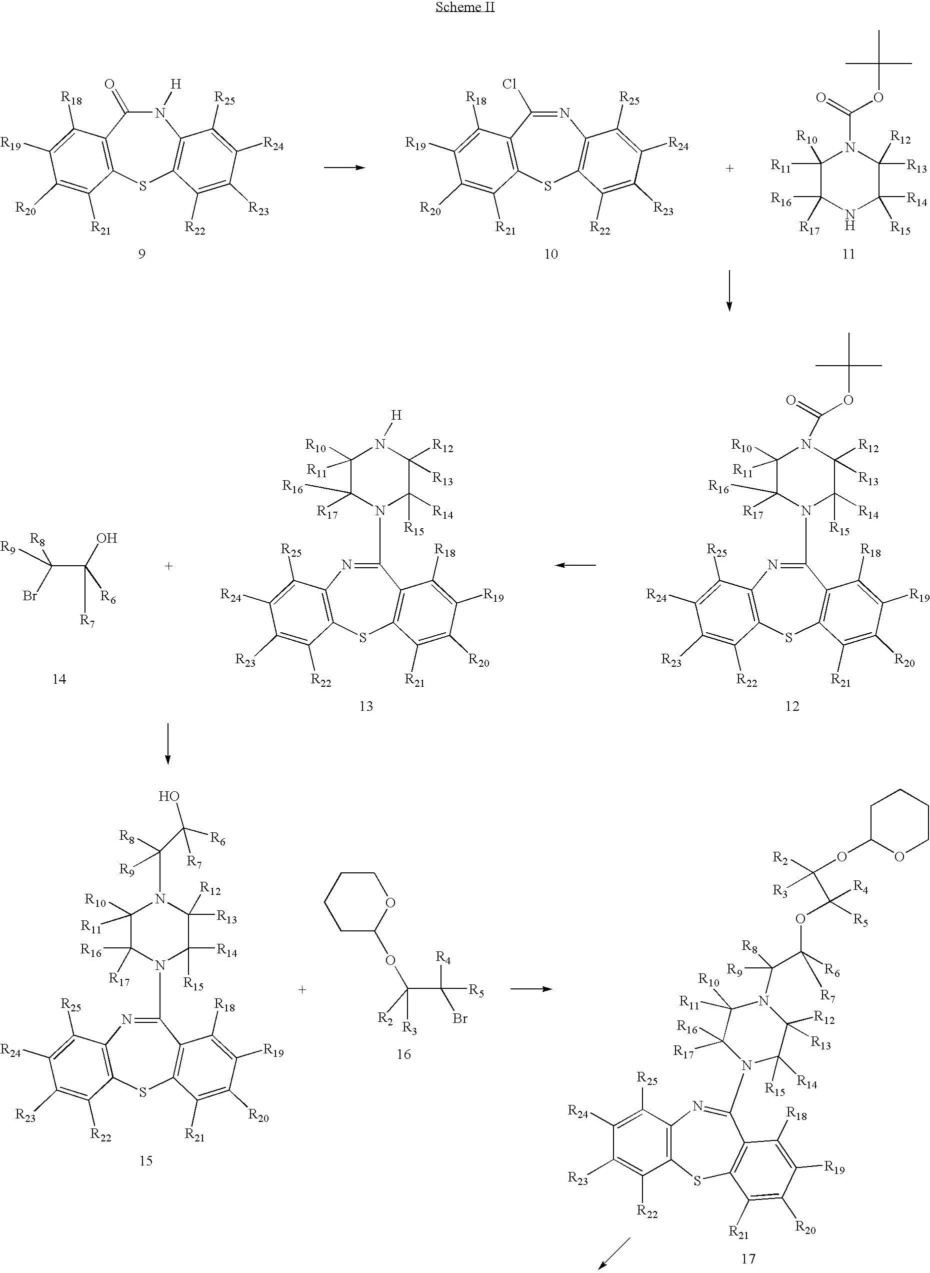

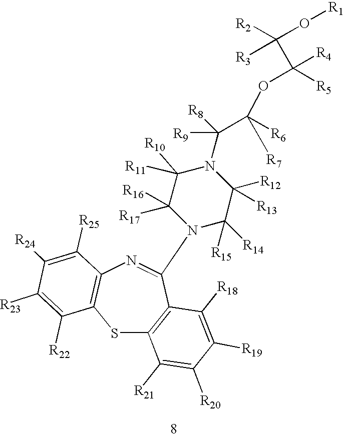

Compound 9 is treated with an appropriate chlorinating agent, such as phosphorous oxychloride, in the presence of an appropriate base, such as dimethyl aniline, at an elevated temperature to give compound 10. Compound 10 is reacted with compound 11 in an appropriate solvent, such as o-xylene, at an elevated temperature to give compound 12. Compound 12 is treated with an appropriate acid, such as hydrochloric acid, in an appropriate solvent, such as methanol, to give compound 13. Compound 13 is reacted with compound 14 in the presence of an appropriate base, such as potassium carbonate, and in the presence of an appropriate catalyst, such as sodium iodide, in an appropriate solvent, such as 1-butanol, at an elevated temperature to give a compound 15. Compound 15 is reacted with compound 16 in the presence of an appropriate base, such as sodium hydroxide, and in the presence of an appropriate phase transfer catalyst, such as tetrabutylammonium hydrogensulfate, at an elevated temperature to give compound 17. Compound 17 is treated with an appropriate acid, such as hydrochloric acid, in appropriate solvents, such as water and toluene or an appropriate mixture thereof, to give compound 8 of Formula I.

Deuterium can be incorporated to different positions synthetically, according to the synthetic procedures as shown in Scheme II, by using appropriate deuterated intermediates. For example, to introduce deuterium at one or more positions of R₁₈-R₂₅, compound 9 with the corresponding deuterium substitutions can be used. To introduce deuterium at one or more positions of R₁₀-R₁₇, compound 11 with the corresponding deuterium substitutions can be used. To introduce deuterium at one or more positions of R₆-R₉, compound 14 with the corresponding deuterium substitutions can be used. To introduce deuterium at one or more positions of R₂-R₅, compound 16 with the corresponding deuterium substitutions can be used.

Deuterium can be incorporated to various positions having an exchangeable proton, such as the hydroxyl O—H, via proton-deuterium equilibrium exchange. For example, to introduce deuterium at R₁, this proton may be replaced with deuterium selectively or non-selectively through a proton-deuterium exchange method known in the art.

The invention is further illustrated by the following examples. All IUPAC names were generated using CambridgeSoft's ChemDraw 10.0.

### EXAMPLE 1

### (E)-2-[2-(4-(Dibenzo[b,f][1,4]thiazepin-11-yl)piperazin-1-yl)ethoxy]ethanol fumarate

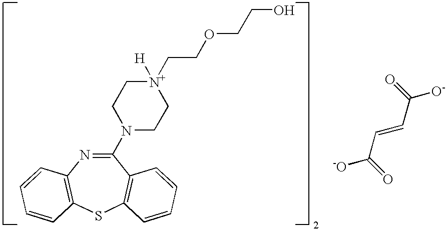

### Step 1

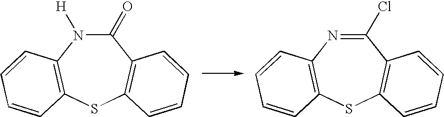

(E)-11-Chlorodibenzo[b,f][1,4]thiazepine: A mixture of dibenzo[b,f][1,4]thiazepin-11(10H)-one (5.00 g, 22.00 mmol), N,N-dimethylaniline (0.83 mL, 6.55 mmol) and phosphorus(V) oxychloride (50 mL) was heated at reflux for about 24 hours. The volatiles were removed in vacuo, and the residue was diluted with cold water and extracted with ethyl acetate. The organic layer was washed with 1N hydrochloric acid and water, and the solvent was evaporated in vacuo to give the title product as a pale yellow solid, which was used in the next step without further purification (5.00 g, 92%). m.p. 240° C.; 1H NMR (400 MHz, CDCl₃) δ 7.13-7.48 (m, 7H), 7.75 (d, J=7.8 Hz, 1H); IR (KBr) ν 3172, 3042, 1650, 1571, 1469, 1433, 1375 cm-1; MS 246, 248 [(M+1), (M+3)].

### Step 2

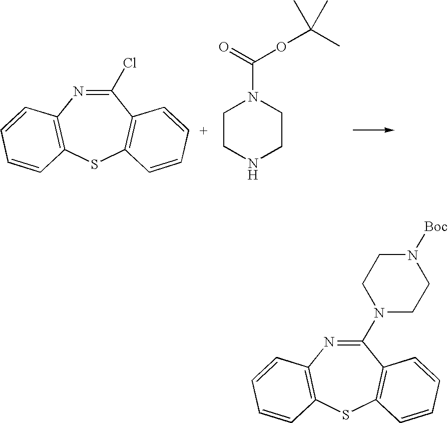

(E)-tert-Butyl 4-(dibenzo[b,f][1,4]thiazepin-11-yl)piperazine-1-carboxylate: A mixture of (E)-11-chlorodibenzo[b,f][1,4]thiazepine (5.00 g, 20.35 mmol), 1-tert-butyl-piperazine-1-carboxylate (5.68 g, 30.50 mmol) and o-xylene (50 mL) was stirred at about 130° C. for about 7 hours. Standard extractive work up provided a crude residue which was purified by silica gel column chromatography (8% ethyl acetate in petroleum ether) to give the title product as an off-white solid (5.0 g, 62%). m.p. 127-130° C.; ¹H NMR (400 MHz, CDCl₃) δ 1.47 (s, 9H), 2.33-2.64 (m, 8H), 6.88-7.37 (m, 6H), 7.39 (d, J=7.8 Hz, 1H), 7.52 (d, J=7.2 Hz, 1H); IR (KBr) ν 2972, 2914, 2852, 1698, 1599, 1410, 1241 cm⁻¹; MS 396 (M+1).

### Step 3

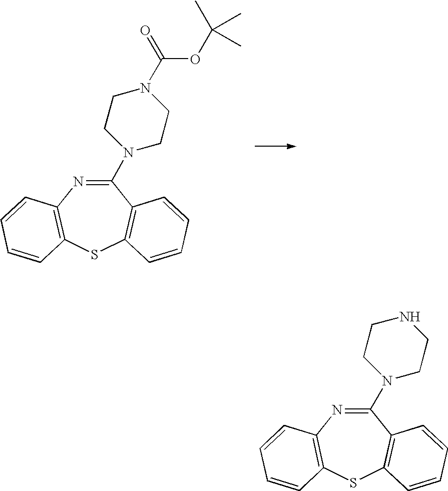

(E)-11-(Piperazin-1-yl)dibenzo[b,f][1,4]thiazepine: Methanol saturated with hydrochloric gas (45 mL) was added to a solution of (E)-tert-butyl 4-(dibenzo[b,f][1,4]thiazepin-11-yl)piperazine-1-carboxylate (4.50 g, 11.38 mmol) in methanol (9 mL). The resulting mixture was stirred at ambient temperature for about 24 hours. The volatiles were removed in vacuo and the resulting residue was basified to pH 9 with a 10% sodium carbonate solution. Standard extractive work up provided a viscous residue which upon trituration with petroleum ether gave the title product as a pale yellow sticky solid (2.70 g, 80%). m.p. 66-69° C.; ¹H NMR (400 MHz, CDCl₃) δ 2.86-3.08 (m, 4H), 3.33-3.64 (m, 4H), 6.87-7.38 (m, 6H), 7.41 (d, J=7.4 Hz, 1H), 7.53 (d, J=7.4 Hz, 1H); IR (film) ν 3052, 2947, 2845, 1600, 1450, 1406, 1301, 1255 cm⁻¹; MS 296 (M+1).

### Step 4

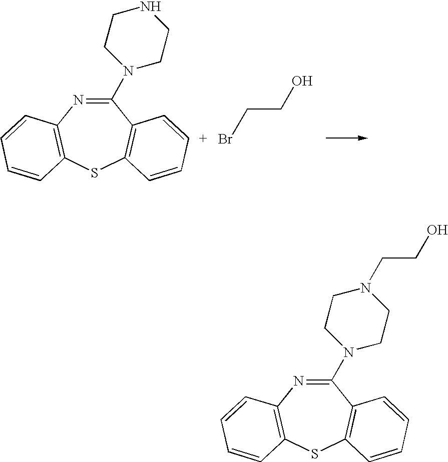

(E)-2-(4-(Dibenzo[b,f][1,4]thiazepin-11-yl)piperazin-1-yl)ethanol: A mixture of (E)-11-(piperazin-1-yl)dibenzo[b,f][1,4]thiazepine (1.00 g, 3.385 mmol), 2-bromoethanol (0.500 g, 4.00 mmol), anhydrous potassium carbonate (0.468 g, 3.386 mmol), sodium iodide (0.250 g, 1.67 mmol) and 1-butanol (20 mL) was heated at reflux for about 24 hours. The reaction mixture was filtered and the solid was washed with methanol. The combined filtrate and washings were concentrated to provide a crude residue. The residue was purified by chromatography on neutral alumina (2% methanol in dichloromethane) to afford the title product as an off-white solid (0.990 g, 86%). m.p. 57-60° C.; ¹H NMR (400 MHz, pyridine-d₅, 60° C.) δ 2.53-2.60 (m, 2H), 2.63-2.70 (m, 2H), 2.67 (t, J=5.9 Hz, 2H), 3.46-3.68 (m, 4H), 3.89 (t, J=5.9 Hz, 2H), 6.86-7.59 (m, 8H); IR (film) ν 3134, 1598, 1405, 1265 cm⁻¹; MS 340 (M+1).

### Step 5

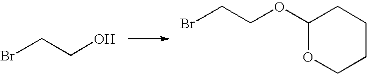

2-(2-Bromoethoxy)tetrahydro-2H-pyran: 3,4-Dihydro-2H-pyran (7.80 g, 92.72 mmol) was added dropwise to a mixture of 2-bromoethanol (10.00 g, 80.03 mmol), p-toluenesulfonic acid monohydrate (1.52 g, 7.99 mmol) and dry dichloromethane (50 mL) at about 0° C. The reaction mixture was stirred at ambient temperature for about 24 hours. The volatiles were removed in vacuo to provide a crude residue. The residue was purified by silica gel column chromatography (diethyl ether) to afford the title product as a dark oil which was used in the next step without further purification (13.00 g, 78%). ¹H NMR (400 MHz, CDCl₃) δ 1.48-1.91 (m, 6H), 3.44-3.55 (m, 3H), 3.72-3.81 (m, 1H), 3.83-3.93 (m, 1H), 3.96-4.04 (m, 1H), 4.67 (t, J=3.6 Hz, 1H); IR (film) ν 2943, 2885, 1448, 1353, 1274 cm⁻¹.

### Step 6

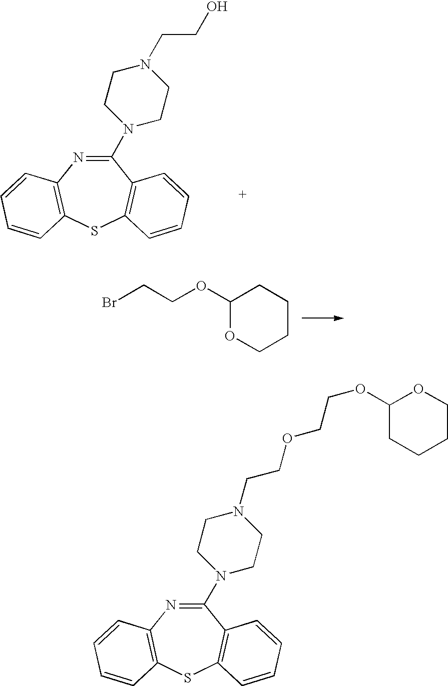

(E)-11-(4-(2-(2-(Tetrahydro-2H-pyran-2-yloxy)ethoxy)ethyl)piperazin-1-yl)dibenzo[b,f][1,4]thiazepine: A mixture of (E)-2-(4-(dibenzo[b,f][1,4]thiazepin-11-yl)piperazin-1-yl)ethanol (1.10 g, 3.24 mmol), 2-(2-bromoethoxy)tetrahydro-2H-pyran (3.05 g, 14.59 mmol), tetrabutylammonium hydrogensulfate (0.110 g, 0.324 mmol) and a 50% sodium hydroxide solution (8.8 mL, 110 mmol) was stirred at about 60° C. for about 24 hours. Standard extractive work up provided a crude residue which was purified by Preparative HPLC on a Kromasil 100 C18 (250×30 mm, 10μ) column (eluting with methanol/0.01M ammonium acetate (90:10) at a flow rate of 42 mL/min). The title compound eluted at 5.53 min. The fractions were basified to pH 9 with a 10% sodium carbonate solution and then extracted with ethyl acetate. The organic layer was concentrated in vacuo to yield the title compound as a pale yellow oil (0.600 g, 40%). ¹H NMR (400 MHz, pyridine-d₅, 60° C.) δ 1.36-1.89 (m, 6H), 2.53-2.72 (m, 6H), 3.47-3.77 (m, 10H), 3.89-4.03 (m, 2H), 4.76 (t, J=3.5 Hz, 1H), 6.91-7.65 (m, 8H); IR (film) ν 3054, 1598, 1414, 1265 cm⁻¹; MS 468 (M+1).

### Step 7

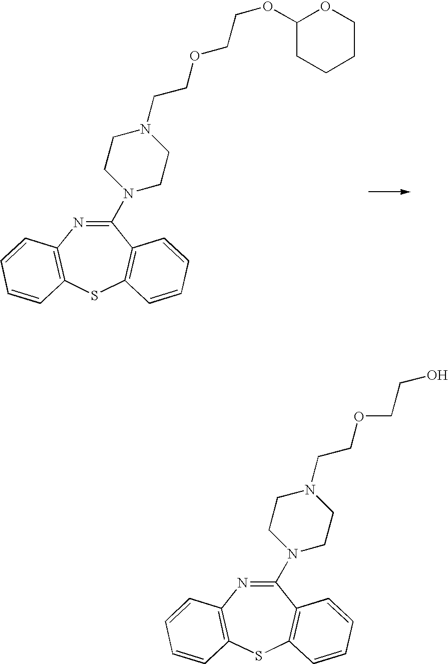

(E)-2-(2-(4-(Dibenzo[b,f][1,4]thiazepin-11-yl)piperazin-1-yl)ethoxy)ethanol: A biphasic mixture of (E)-11-(4-(2-(2-(tetrahydro-2H-pyran-2-yloxy)ethoxy)ethyl)piperazin-1-yl)dibenzo[b,f][1,4]thiazepine (0.400 g, 0.855 mmol), toluene (2.64 mL), water (1.84 mL) and conc. hydrochloric acid (0.28 mL) was stirred vigorously at ambient temperature for about 4 hours. The layers were separated and the organic layer was discarded. The aqueous layer was basified to pH 9 with a 10% potassium carbonate solution. Standard extractive work up provided a crude residue which was purified by Preparative HPLC on a Kromasil 100 C18 (250×20 mm, 10μ) column, (eluting with acetonitrile/0.1% formic acid (gradient) at a flow rate of 20 mL/min). The title compound eluted at 4.47 min. The fractions were basified to pH 9 with a 10% sodium carbonate solution and then extracted with ethyl acetate. The organic layer was concentrated in vacuo to yield the title compound as an oil (0.255 g, 78%). ¹H NMR (400 MHz, pyridine-d₅, 60° C.) δ 2.49-2.68 (m, 6H), 3.46-3.57 (m, 2H), 3.60-3.70 (m, 6H), 3.91 (t, J=5.0 Hz, 2H), 6.85-7.59 (m, 8H); IR (film) ν 3131, 1601, 1404, 1265 cm⁻¹; MS 384 (M+1).

### Step 8

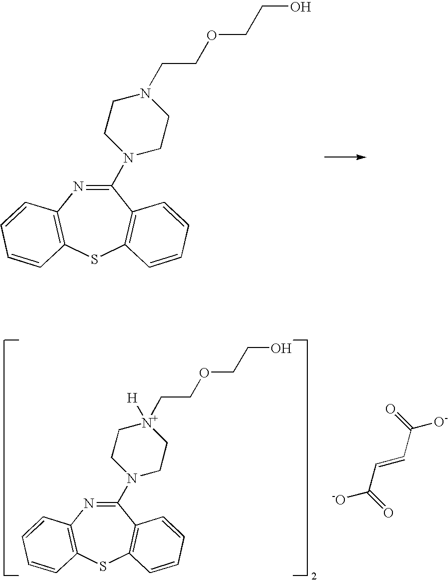

(E)-2-(2-(4-(Dibenzo[b,f][1,4]thiazepin-11-yl)piperazin-1-yl)ethoxy)ethanol fumarate: A solution of fumaric acid (0.042 g, 0.353 mmol) in ethanol (3 mL) was added dropwise to a solution of (E)-2-(2-(4-(dibenzo[b,f][1,4]thiazepin-11-yl)piperazin-1-yl)ethoxy)ethanol (0.275 g, 0.717 mmol) in ethanol (4 mL). The resulting mixture was heated at reflux for about 2 hours, and then cooled slowly to ambient temperature. The resulting precipitate was filtered, washed sequentially with cold ethanol, ether and pentane, and dried to give the title compound as a white solid (0.250 g, 79%). m.p. 174-176° C.; ¹H NMR (400 MHz, pyridine-d₅, 60° C.) δ 2.46-2.67 (m, 6H), 3.45-3.72 (m, 8H), 3.93 (t, J=5.0 Hz, 2H), 6.84-7.62 (m, 9H); IR (film) ν 3432, 3324, 2934, 2864, 1601, 1457, 1414 cm⁻¹; MS 384 [(M+1)-fumaric acid].

### EXAMPLE 2

### (E)-2-[2-(4-(Dibenzo[b,f][1,4]thiazepin-11-yl)piperazin-1-yl)ethoxy-1,1,2,2-d₄]ethanol fumarate

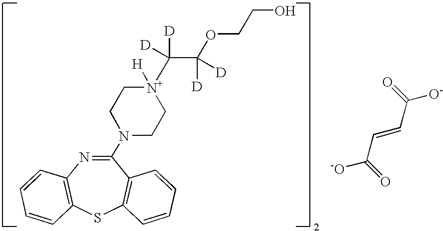

### Step 1

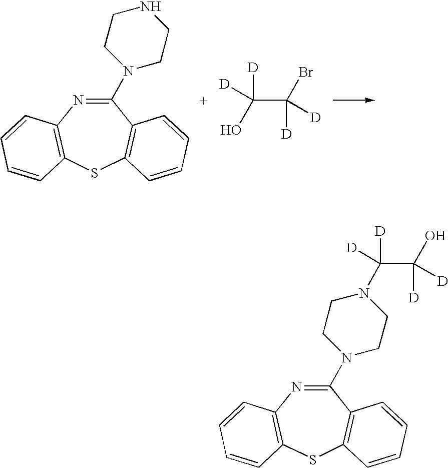

(E)-2-(4-(Dibenzo[b,f][1,4]thiazepin-11-yl)piperazin-1-yl)ethanol-1,1,2,2-D₄: The procedure of Example 1 Step 4 was followed, but substituting bromoethanol-1,1,2,2-D₄ for bromoethanol. The title compound was isolated as an off-white sticky solid (0.380 g, 65%). m.p. 55° C.; ¹H NMR (400 MHz, pyridine-d₅, 60° C.) δ 2.52-2.59 (m, 2H), 2.61-2.70 (m, 2H), 3.49-3.69 (m, 4H), 6.86-7.59 (m, 8H); IR (film) ν 3423, 2806, 1596, 1450, 1406, 1300, 1254 cm⁻¹; MS 344 (M+1).

### Step 2

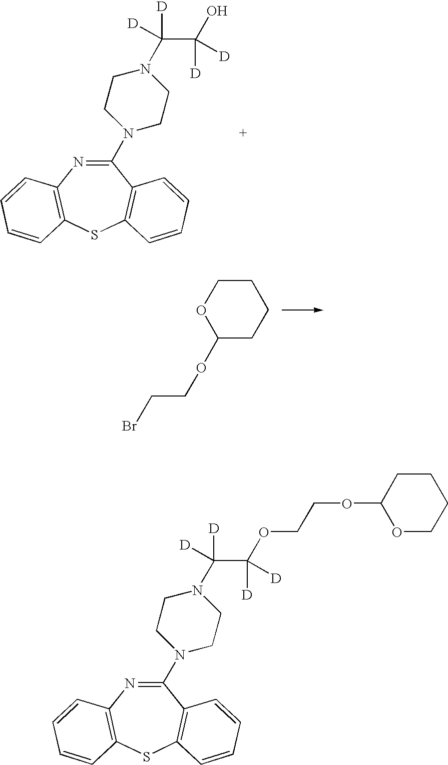

(E)-11-(4-(2-(2-(Tetrahydro-2H-pyran-2-yloxy)ethoxy)ethyl-d₄)piperazin-1-yl)dibenzo[b,f][1,4]thiazepine: The procedure of Example 1, Step 6 was followed but substituting (E)-2-(4-(dibenzo[b,f][1,4]thiazepin-11-yl)piperazin-1-yl)ethanol-1,1,2,2-d₄ for (E)-2-(4-(dibenzo[b,f][1,4]thiazepin-11-yl)piperazin-1-yl)ethanol. The title compound was isolatd as a pale yellow oil (0.380 g, 69%). ¹H NMR (400 MHz, pyridine-d₅, 60° C.) δ 1.32-1.89 (m, 6H), 2.49-2.68 (m, 4H), 3.43-3.70 (m, 8H), 3.83-3.99 (m, 2H), 4.71 (t, J=3.5 Hz, 1H), 6.86-7.60 (m, 8H); IR (film) ν 2944, 1599, 1414, 1266 cm⁻¹; MS 472 (M+1).

### Step 3

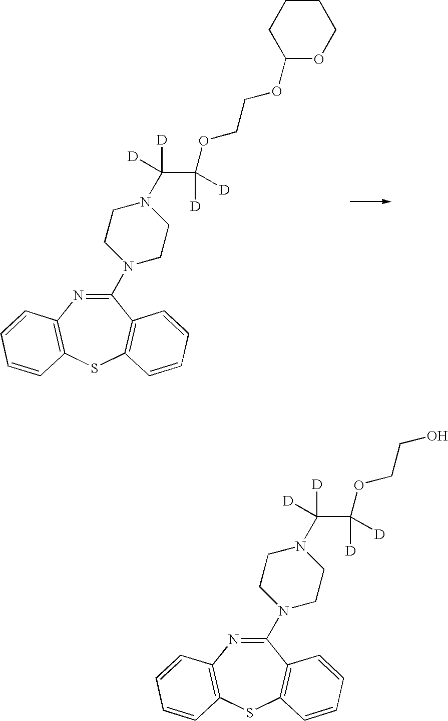

E)-2-(2-(4-(Dibenzo[b,f][1,4]thiazepin-11-yl)piperazin-1-yl)ethoxy-d₄)ethanol: The procedure of Example 1 Step 7 was followed but substituting (E)-11-(4-(2-(2-(tetrahydro-2H-pyran-2-yloxy)ethoxy)ethyl-d₄)piperazin-1-yl)dibenzo[b,f][1,4]thiazepine for (E)-11-(4-(2-(2-(tetrahydro-2H-pyran-2-yloxy)ethoxy)ethyl)piperazin-1-yl)dibenzo[b,f][1,4]thiazepine. The title compound was isolated as an oil, which was used in the next step without further purification (0.250 g, 76%). ¹H NMR (400 MHz, pyridine-d₅, 60° C.) δ 2.52-2.72 (m, 4H), 3.48-3.77 (m, 4H), 3.74 (t, J=5.1 Hz, 2H), 3.98 (t, J=5.1 Hz, 2H), 6.91-7.65 (m, 8H); IR (film) ν 3391, 2933, 2855, 1600, 1401, 1303, 1260 cm⁻¹; MS 388 (M+1).

### Step 4

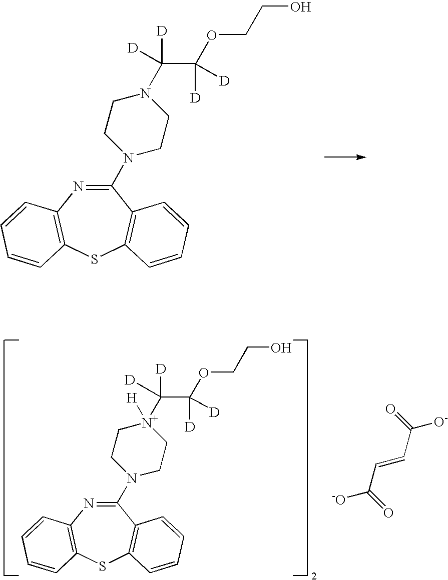

(E)-2-(2-(4-(Dibenzo[b,f][1,4]thiazepin-11-yl)piperazin-1-yl)ethoxy-d₄)ethanol fumarate: The procedure of Example 1 Step 8 was followed but substituting (E)-2-(2-(4-(dibenzo[b,f][1,4]thiazepin-11-yl)piperazin-1-yl)ethoxy-d₄)ethanol for (E)-2-(2-(4-(dibenzo[b,f][1,4]thiazepin-11-yl)piperazin-1-yl)ethoxy)ethanol. The title compound was isolated as a white solid (0.100 g, 58%). m.p. 172-175° C.; ¹H NMR (400 MHz, pyridine-d₅, 60° C.) δ 2.49-2.67 (m, 4H), 3.48-3.72 (m, 4H), 3.69 (t, J=5.1 Hz, 2H), 3.93 (t, J=5.1 Hz, 2H), 6.84-7.61 (m, 9H); IR (film) ν 3448, 3328, 2934, 1601, 1457, 1416, 1333 cm⁻¹; MS 388 [(M+1)-fumaric acid].

### EXAMPLE 3

(E)-2-[2-(4-(Dibenzo[b,f][1,4]thiazepin-11-yl)piperazin-1-yl)ethoxy-1,1,2,2-d₄]ethanol-1,1,2,2-d₄ fumarate

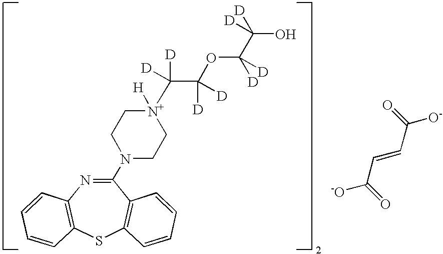

### Step 1

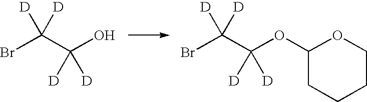

2-(2-Bromoethoxy-d₄)tetrahydro-2H-pyran: The procedure of Example 1 Step 5 was followed but substituting 2-bromoethanol-1,1,2,2-d₄ (1.00 g, 7.75 mmol) for 2-bromo ethanol. The title product was isolated as a dark oil which was not purified further (1.10 g, 67%). ¹H NMR (400 MHz, CDCl₃) δ 1.47-1.91 (m, 6H), 3.48-3.57 (m, 1H), 3.83-3.94 (m, 1H), 4.67 (t, J=3.6 Hz, 1H); IR (film) ν 2944, 2863, 1446, 1382, 1268 cm⁻¹.

### Step 2

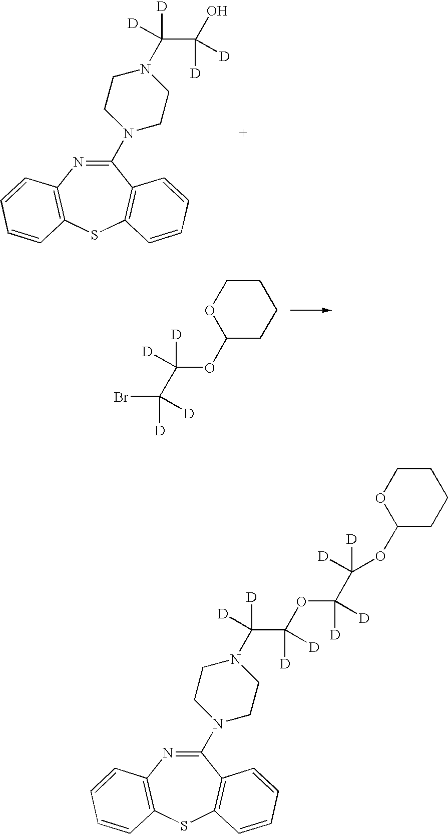

(E)-11-(4-(2-(2-(Tetrahydro-2H-pyran-2-yloxy)ethoxy-d₄)ethyl-d₄)piperazin-1-yl)dibenzo[b,f][1,4]thiazepine: The procedure of Example 2 Step 2 was followed but substituting 2-(2-bromoethoxy-d₄)tetrahydro-2H-pyran for 2-(2-bromoethoxy-d₄)tetrahydro-2H-pyran. The title compound was isolated as a pale yellow oil (0.300 g, 54%). ¹H NMR (400 MHz, pyridine-d₅, 60° C.) δ 1.30-1.84 (m, 6H), 2.49-2.68 (m, 4H), 3.43-3.70 (m, 5H), 3.85-3.94 (m, 1H), 4.71 (t, J=3.5 Hz, 1H), 6.86-7.60 (m, 8H); IR (film) ν 2933, 2855, 1596, 1451, 1404 cm⁻¹; MS 476 (M+1).

### Step 3

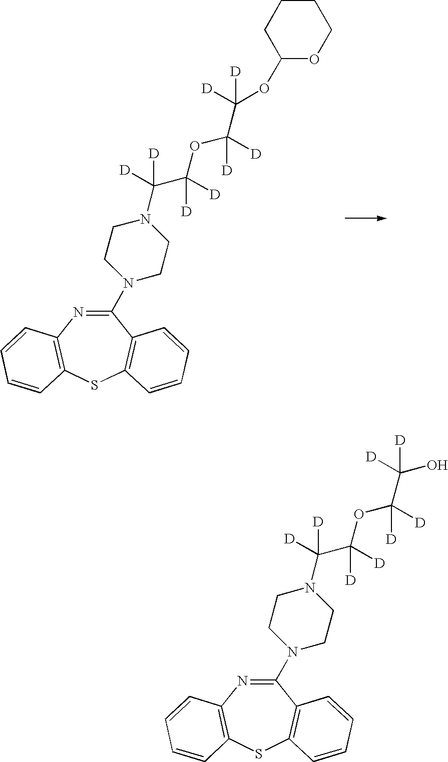

(E)-2-(2-(4-(Dibenzo[b,f][1,4]thiazeepin-11-yl)piperazin-1-yl)ethoxy-d₄)ethanol-1,1,2,2-d₄: The procedure of Example 2, Step 3 was followed but substituting (E)-11-(4-(2-(2-(tetrahydro-2H-pyran-2-yloxy)ethoxy-d₄)ethyl-d₄)piperazin-1-yl)dibenzo[b,f][1,4]thiazepine for (E)-11-(4-(2-(2-(tetrahydro-2H-pyran-2-yloxy)ethoxy-d₄)ethyl)piperazin-1-yl)dibenzo[b,f][1,4]thiazepine. The title compound was isolated as an oil, which was used in the next step without further purification (0.170 g, 69%). ¹H NMR (400 MHz, pyridine-d₅, 65° C.) δ 2.44-2.68 (m, 4H), 3.42-3.71 (m, 4H), 6.83-7.62 (m, 8H); IR (film) ν 3133, 1600, 1404, 1304, 1259 cm⁻¹; MS 392 (M+1).

### Step 4

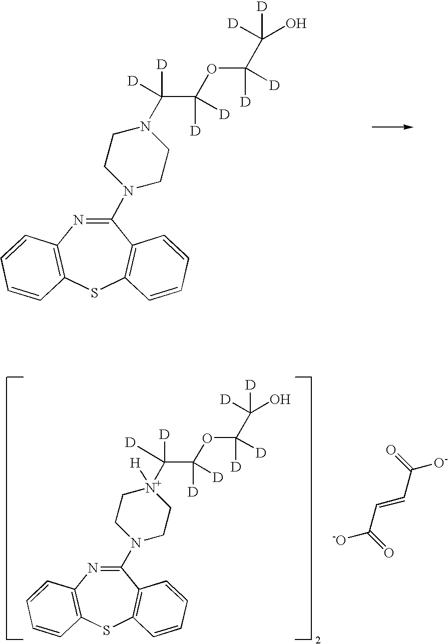

(E)-2-(2-(4-(Dibenzo[b,f][1,4]thiazepin-11-yl)piperazin-1-yl)ethoxy-d₄)ethanol-1,1,2,2-d₄ fumarate: The procedure of Example 2 Step 4 was followed, but substituting (E)-2-(2-(4-(dibenzo[b,f][1,4]thiazepin-11-yl)piperazin-1-yl)ethoxy-d₄)ethanol-1,1,2,2-d₄ for (E)-2-(2-(4-(dibenzo[b,f][1,4]thiazepin-11-yl)piperazin-1-yl)ethoxy-d₄)ethanol. The title compound was isolated as a white solid (0.060 g, 52%). m.p. 172-175° C.; ¹H NMR (400 MHz, pyridine-d₅, 65° C.) δ 2.46-2.69 (m, 4H), 3.45-3.72 (m, 4H), 6.82-7.60 (m, 9H); IR (film) ν 3436, 3329, 1600, 1457, 1414, 1333 cm⁻¹; MS 392 [(M+1)-fumaric acid].

The following compounds can generally be made using the methods described above. It is expected that these compounds when made will have activity similar to those described in the examples above.

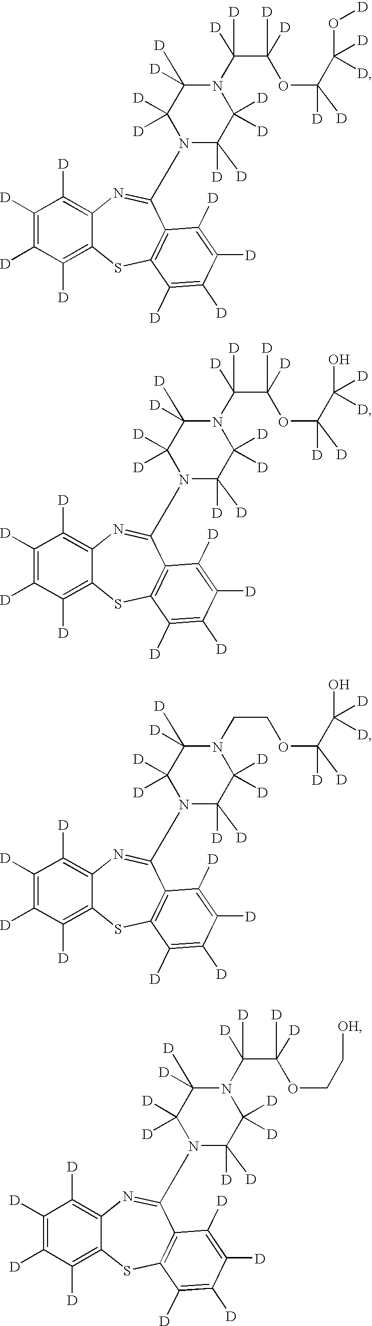

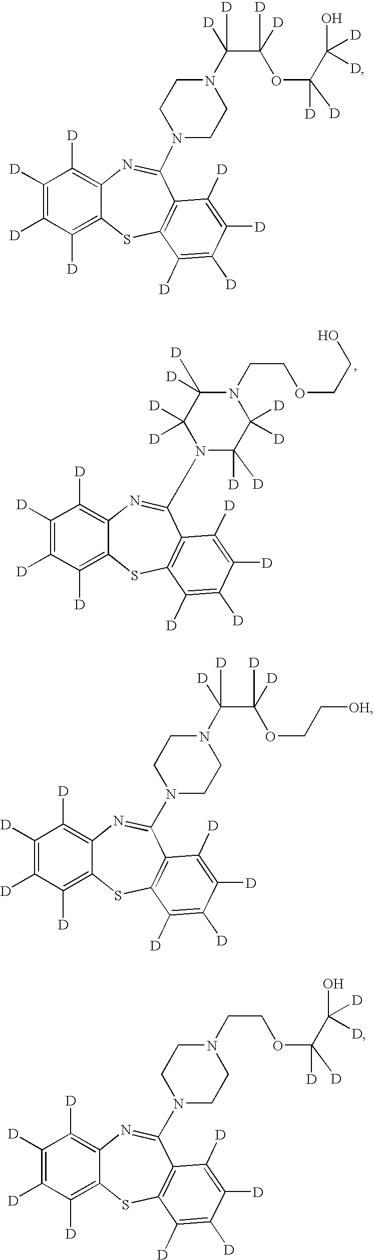

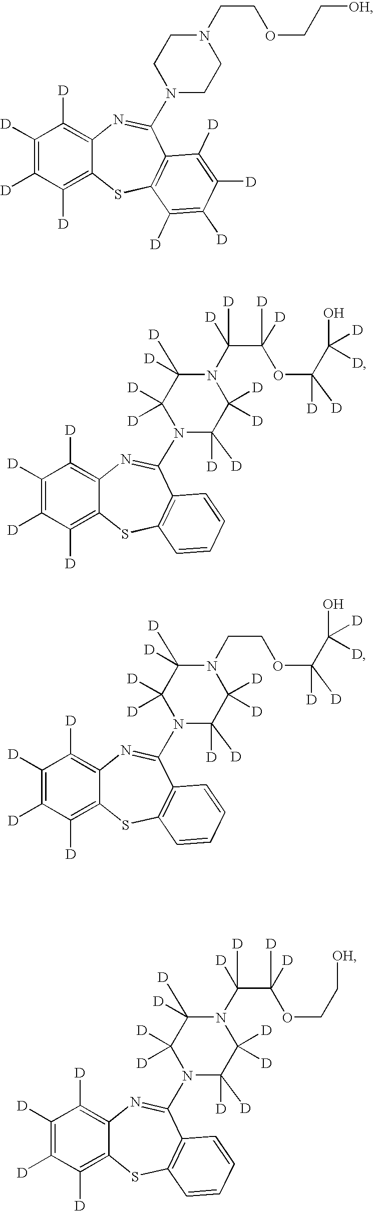

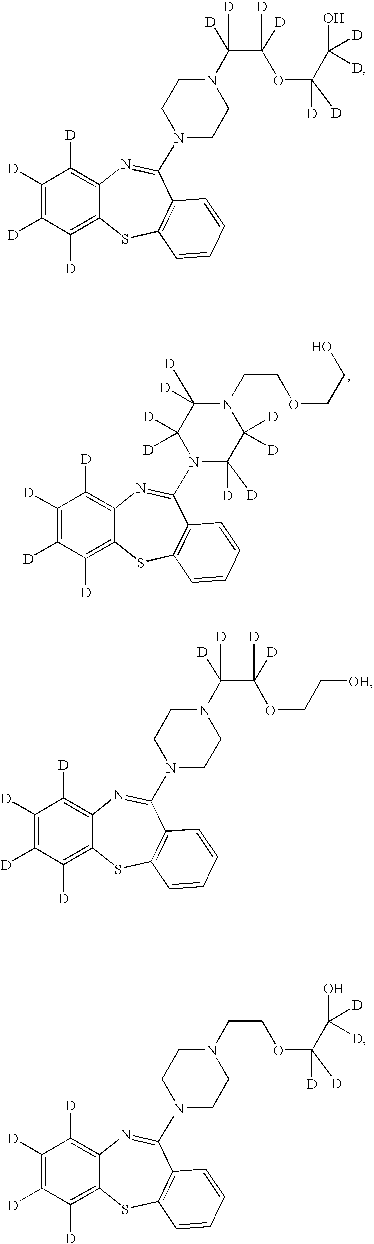

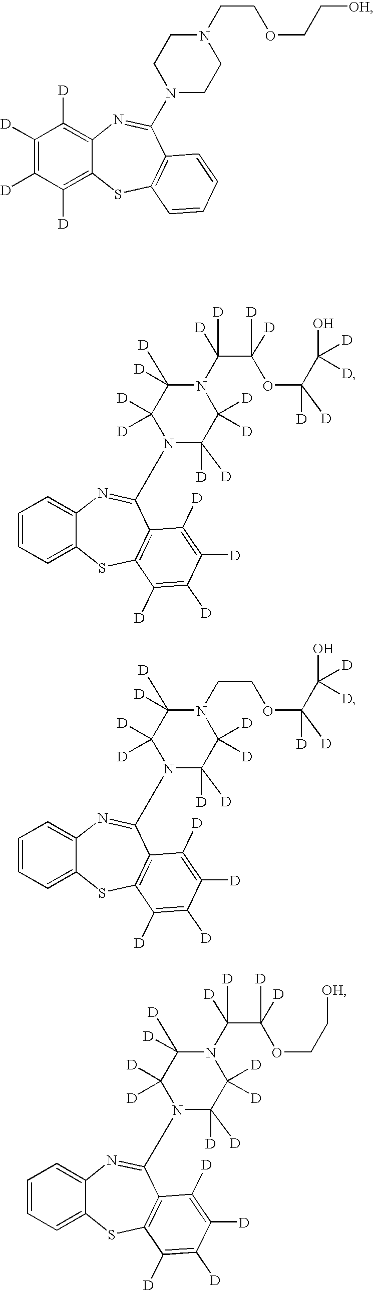

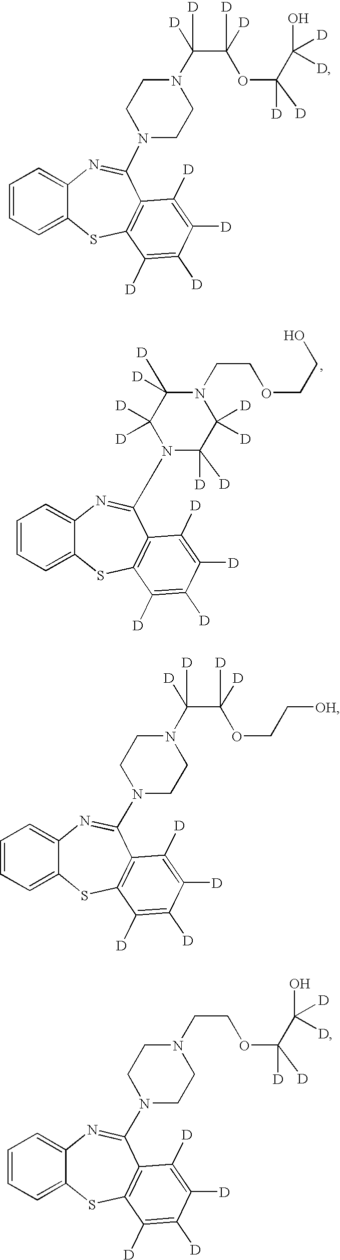

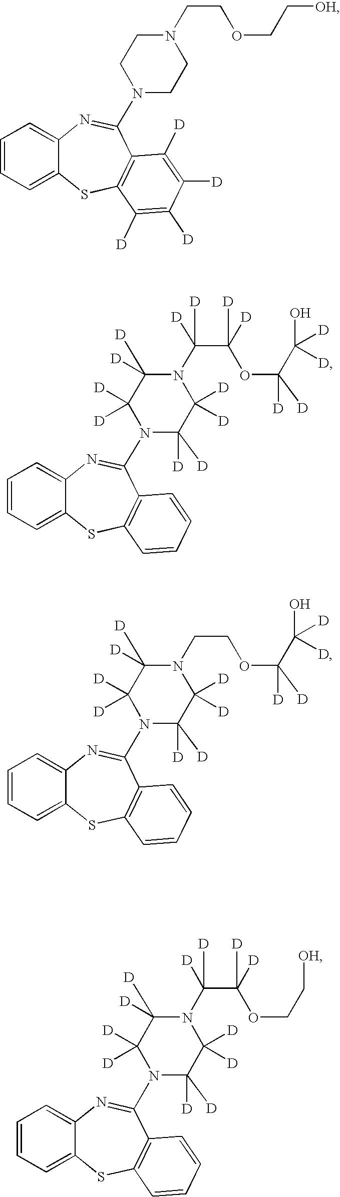

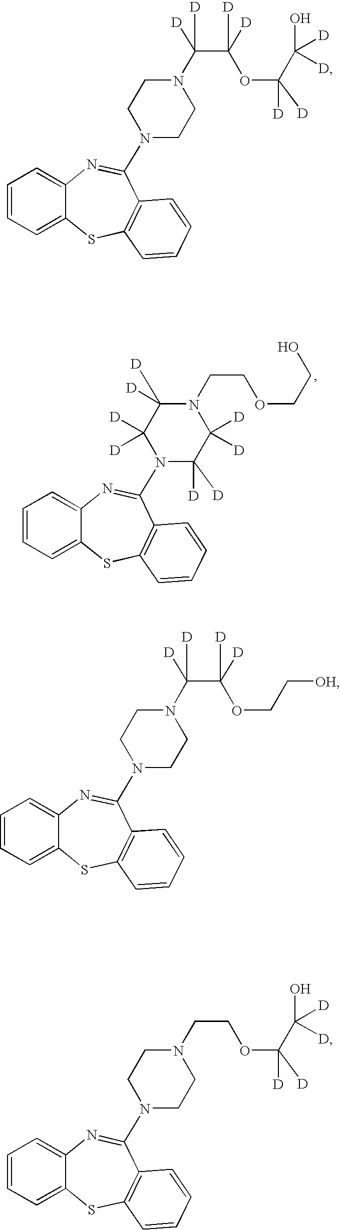

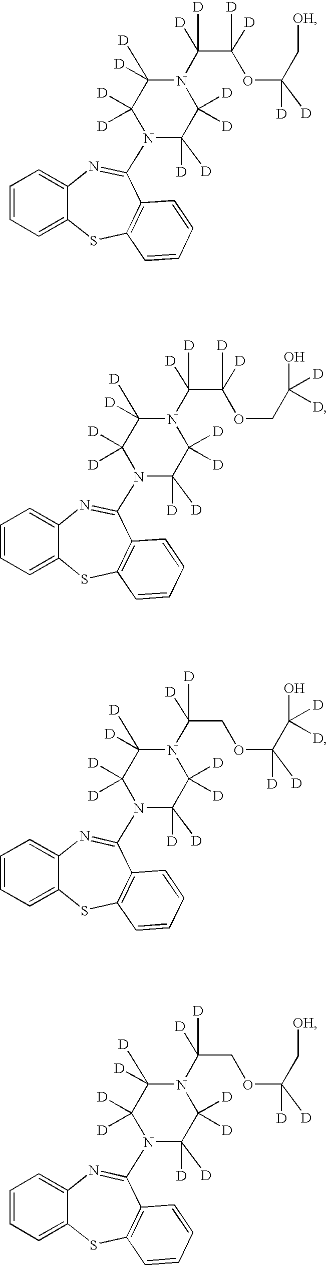

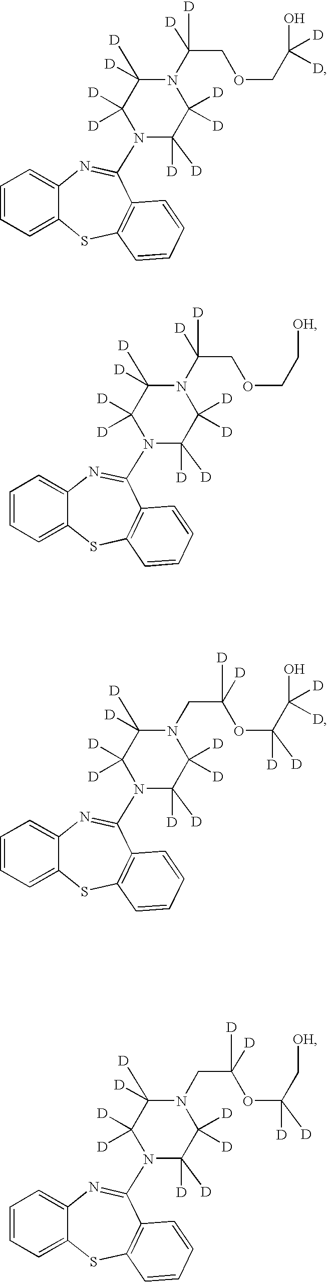

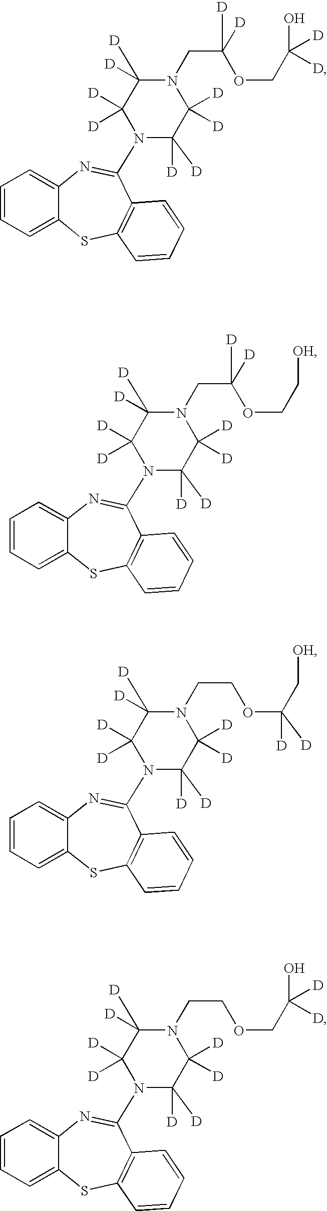

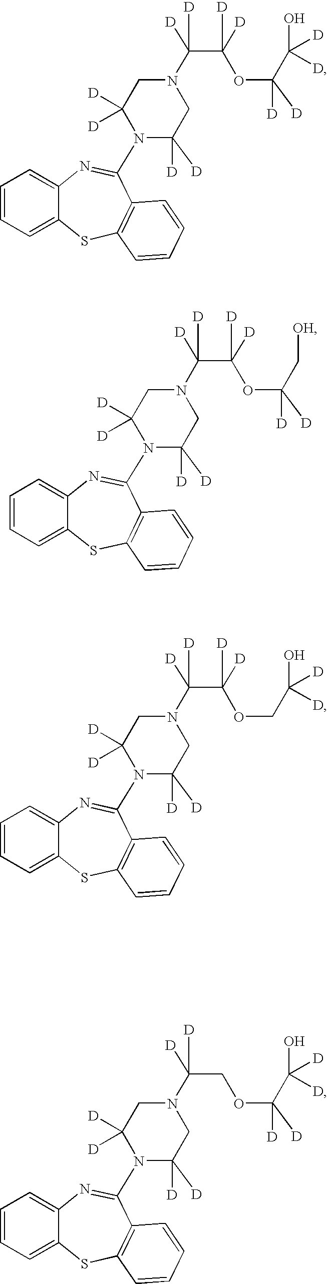

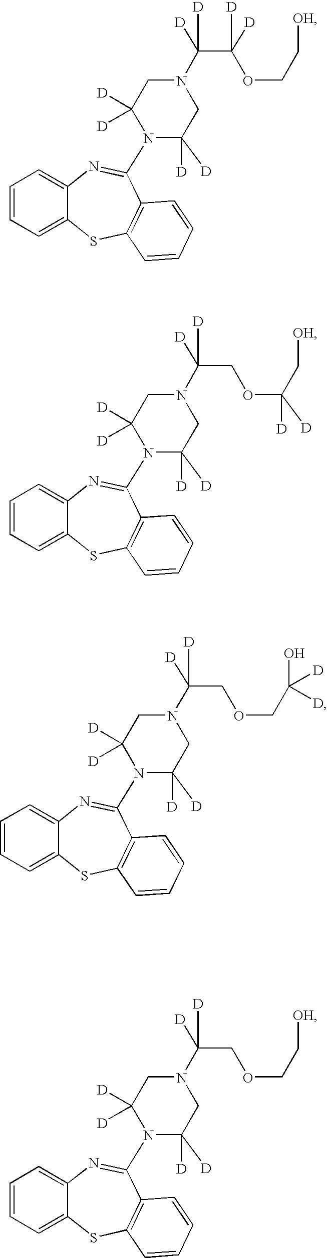

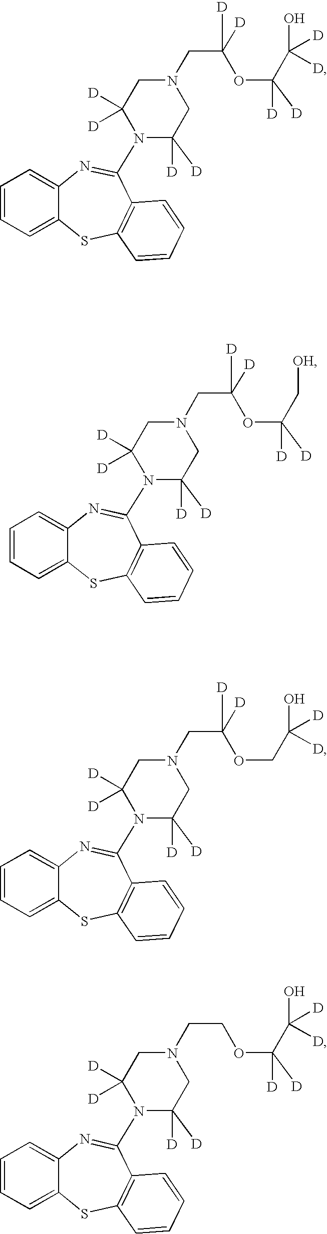

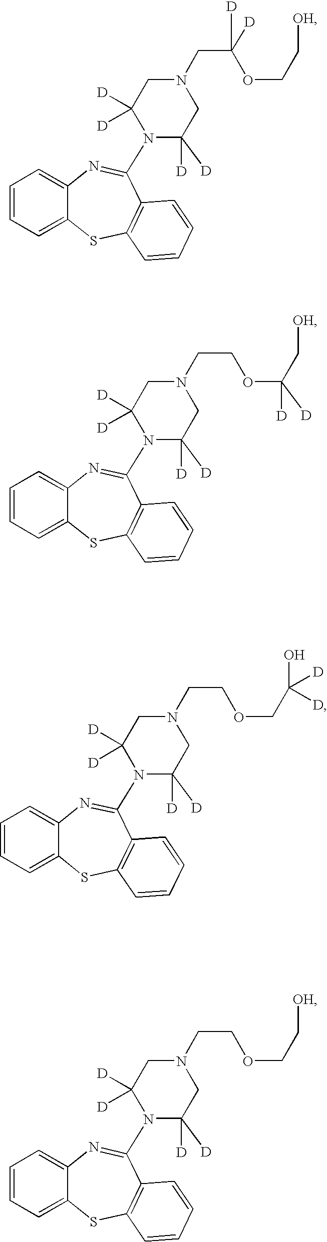

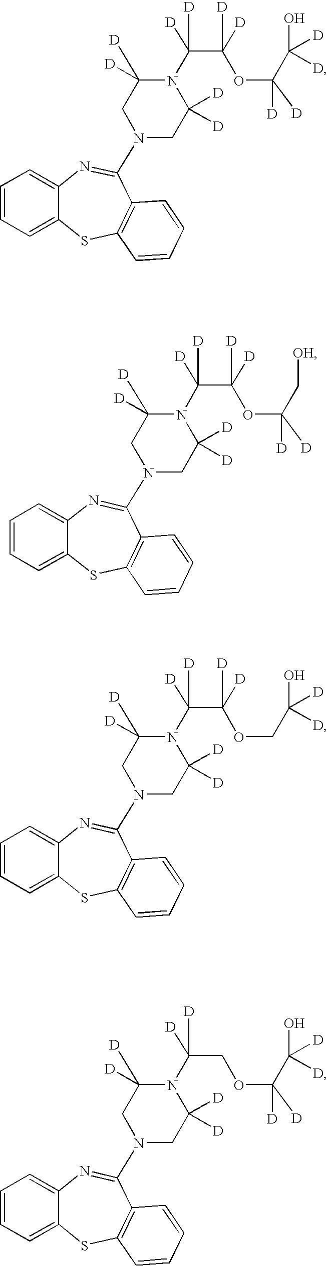

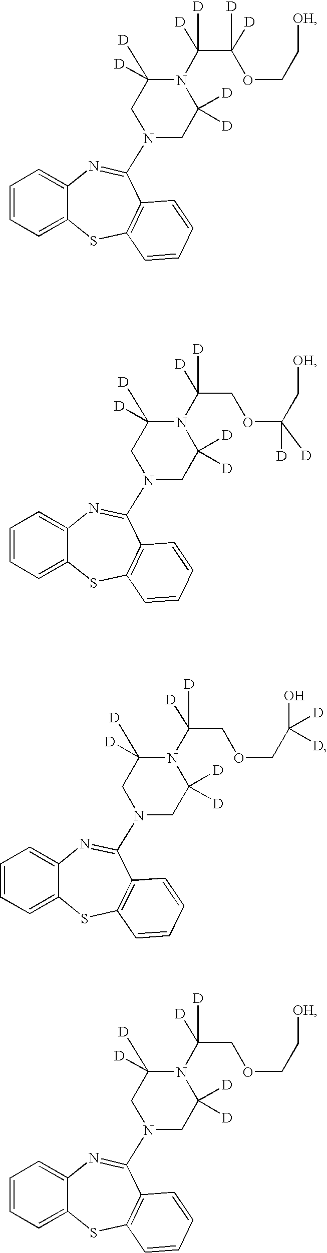

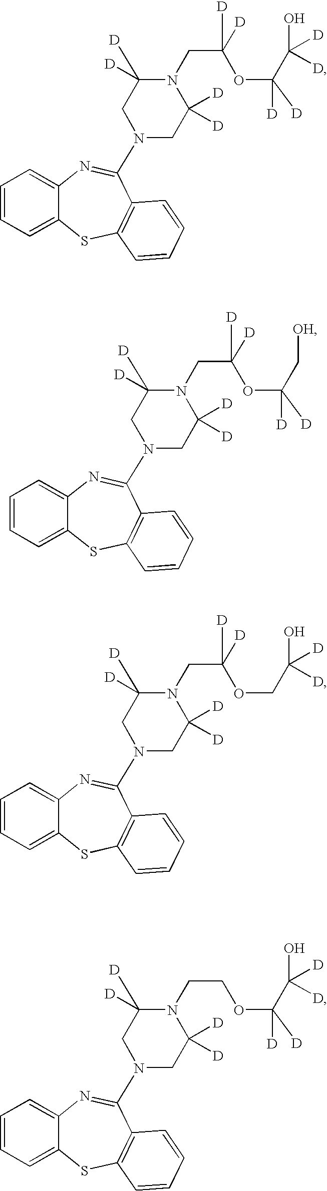

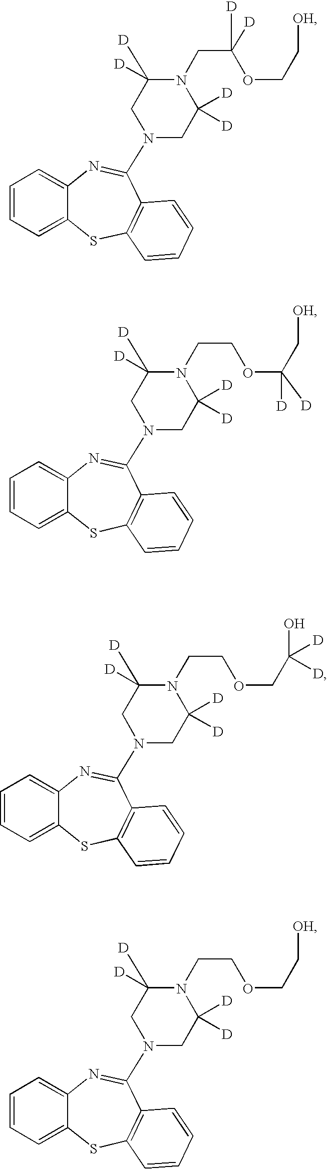

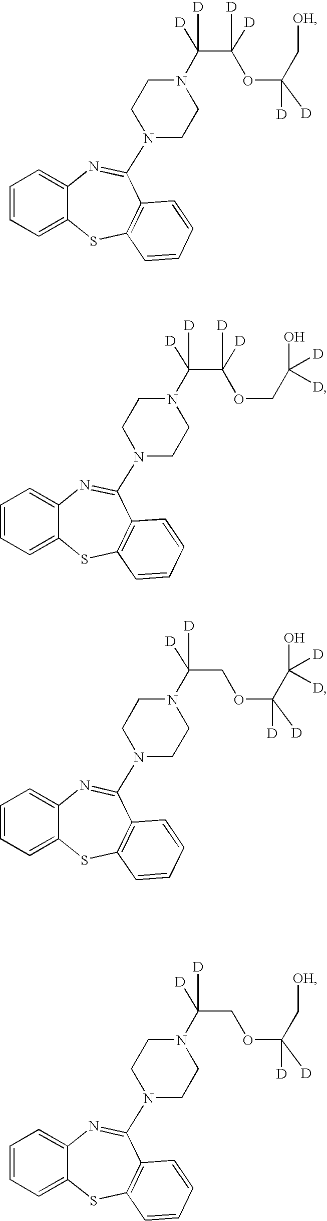

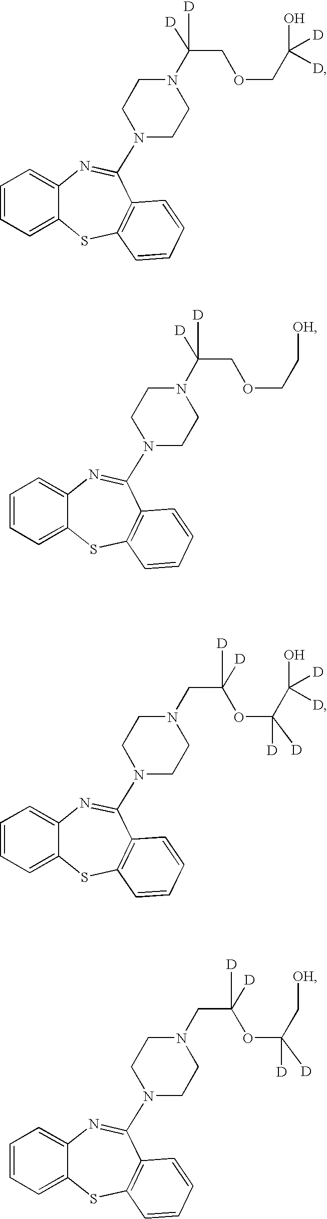

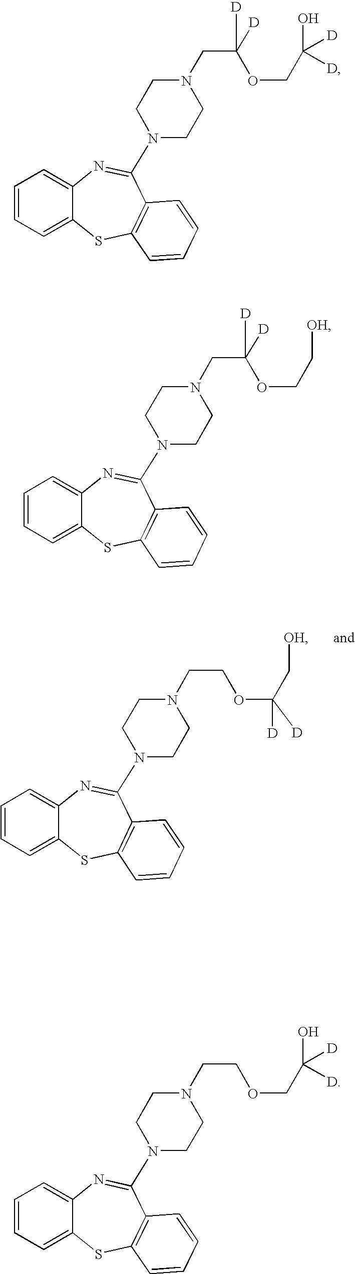

Changes in the metabolic properties of the compounds disclosed herein as compared to their non-isotopically enriched analogs can be shown using the following assays. Compounds listed above which have not yet been made and/or tested are predicted to have changed metabolic properties as shown by one or more of these assays as well.

### Biological Activity Assays

In vitro Liver Microsomal Stability Assay

Liver microsomal stability assays were conducted at 0.5 mg per mL liver microsome protein with an NADPH-generating system in 2% NaHCO₃ (2.2 mM NADPH, 25.6 mM glucose 6-phosphate, 6 units per mL glucose 6-phosphate dehydrogenase and 3.3 mM MgCl₂). Test compounds were prepared as solutions in 20% acetonitrile-water and added to the assay mixture (final assay concentration 1 μM) and incubated at 37° C. Final concentration of acetonitrile in the assay should be <1%. Aliquots (50 μL) were taken out at times 0, 0.125, 0.25, 0.375, and 0.5 hours, and diluted with ice cold acetonitrile (200 μL) to stop the reactions. Samples were centrifuged at 12,000 RPM for 10 min to precipitate proteins. Supernatants were transferred to micro centrifuge tubes and stored for LC/MS/MS analysis of the degradation half-life of the test compounds. It has thus been found that the compounds as disclosed herein according to the present invention that have been tested in this assay showed improved degradation half-life, as compared to the non-isotopically enriched drug. Some of the compounds showed an increase of degradation half-life, as compared to the non-isotopically enriched drug. The degradation half-lives of Examples 1 through 3 (Quetiapine and isotopically enriched drugs) are shown in Table 1.

TABLE 1
Results of in vitro human liver microsomal (HLM) stability assay

% increase of HLM degradation half-life

| | −25%-0% | 0%-50% | 50%-150% | >150% |
|---|---|---|---|---|
| Example 1 | + | | | |
| Example 2 | | + | | |
| Example 3 | | + | | |

### In Vitro Metabolism Using Human Cytochrome P₄₅₀ Enzymes

The cytochrome P₄₅₀ enzymes are expressed from the corresponding human cDNA using a baculovirus expression system (BD Biosciences, San Jose, Calif.). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound of Formula I, the corresponding non-isotopically enriched compound or standard or control in 100 millimolar potassium phosphate (pH 7.4) is incubated at 37° C. for 20 min. After incubation, the reaction is stopped by the addition of an appropriate solvent (e.g., acetonitrile, 20% trichloroacetic acid, 94% acetonitrile/6% glacial acetic acid, 70% perchloric acid, 94% acetonitrile/6% glacial acetic acid) and centrifuged (10,000 g) for 3 min. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]-(S)-mephenytoin |
| CYP2C8 | Paclitaxel |
| CYP2C9 | Diclofenac |
| CYP2C19 | [¹³C]-(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

### Monoamine Oxidase A Inhibition and Oxidative Turnover

The procedure is carried out using the methods described by Weyler, Journal of Biological Chemistry 1985, 260, 13199-13207, which is hereby incorporated by reference in its entirety. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM NaPᵢ buffer, pH 7.2, containing 0.2% Triton X-100 (monoamine oxidase assay buffer), plus 1 mM kynuramine, and the desired amount of enzyme in 1 mL total volume.

### Monooamine Oxidase B Inhibition and Oxidative Turnover

The procedure is carried out as described in Uebelhack, Pharmacopsychiatry 1998, 31(5), 187-192, which is hereby incorporated by reference in its entirety.

### CYP3A4 and CYP3A5 Assays

The procedure is carried out as described in Bakken et al., Drug Metabolism and Disposition 2009, 37(2), 254-258, which is hereby incorporated by reference in its entirety.

### Stability-Indicating HPLC Method for the Determination of Ouetiapine

The procedure is carried out as described in Belal et al., Journal of Liquid Chromatography & Related Technologies 2008, 31(9), 1283-1298, which is hereby incorporated by reference in its entirety.

### Determination of Plasma Concentration of Quetiapine by HPLC-ESI-MS/MS

The procedure is carried out as described in Wen et al., Zhongguo Yaofang 2007, 18(32), 2504-2506, which is hereby incorporated by reference in its entirety.

### Validated HPLC-MS/MS Method for Determination of Quetiapine in Human Plasma

The procedure is carried out as described in Barret et al., Journal of Pharmaceutical and Biomedical Analysis 2007, 44(2), 498-505, which is hereby incorporated by reference in its entirety.

### Measuring Quetiapine Effects on Depressive and Anxiolytic-Like Behavioral Changes Induced by Global Cerebral Ischemia in Mice

The procedure is carried out as described in Yan et al., Behavioural Brain Research 2007, 182(1), 36-41, which is hereby incorporated by reference in its entirety.

Measuring Quetiapine Behavioral Effects with Tremulous Jaw Movement Studies in Rats

The procedure is carried out as described in Betz et al., Psychopharmacology 2005, 179(2), 383-392, which is hereby incorporated by reference in its entirety.

### Selective Antidopaminergic Effects of S(+)N-n-propylnoraporphines in Limbic Versus Extrapyramidal Sites in Rat Brain

The procedure is carried out as described in Campbell et al., Psychopharmacology 103(3) 323-29 (1993), which is hereby incorporated by reference in its entirety.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of structural Formula I 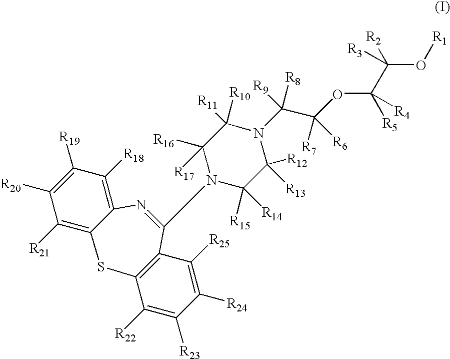
or a salt, solvate, or prodrug thereof, wherein:
R₁-R₂₅ are independently selected from the group consisting of hydrogen and deuterium;
at least one of R₁-R₂₅ is deuterium;
if R₂-R₅ are deuterium, then at least one of R₁ and R₆-R₂₅ is deuterium;
if R₂-R₉ are deuterium, then at least one of R₁ and R₁₀-R₂₅ is deuterium; and
if R₆-R₉ are deuterium, then at least one of R₁-R₅ and R₁₀-R₂₅ is deuterium.

2. The compound as recited in claim 1 wherein at least one of R₁-R₂₅ independently has deuterium enrichment of no less than about 10%.

3. The compound as recited in claim 1 wherein at least one of R₁-R₂₅ independently has deuterium enrichment of no less than about 50%.

4. The compound as recited in claim 1 wherein at least one of R₁-R₂₅ independently has deuterium enrichment of no less than about 90%.

5. The compound as recited in claim 1 wherein at least one of R₁-R₂₅ independently has deuterium enrichment of no less than about 98%.

6. The compound as recited in claim 1 wherein said compound has a structural formula selected from the group consisting of:

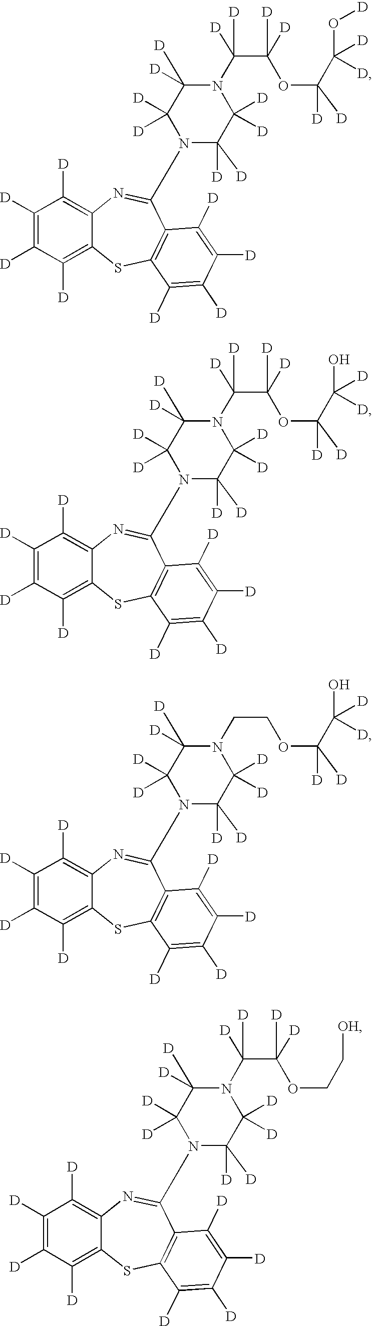

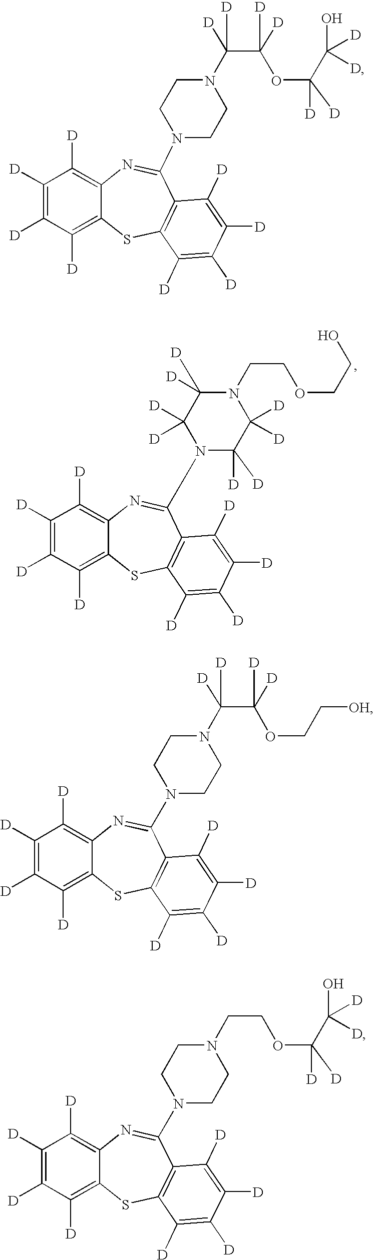

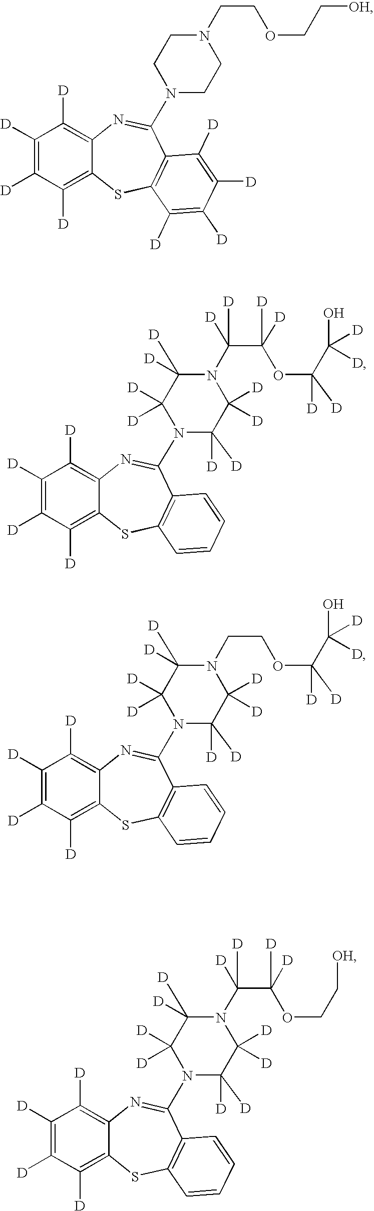

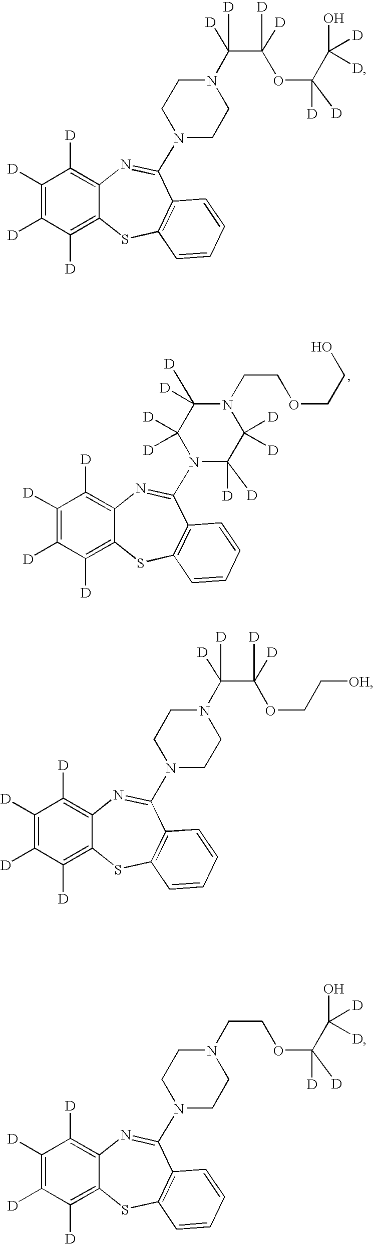

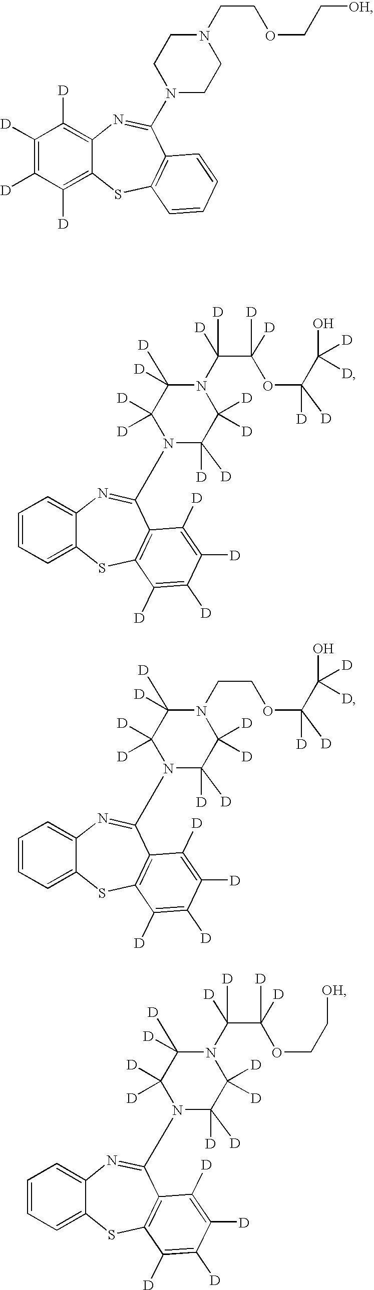

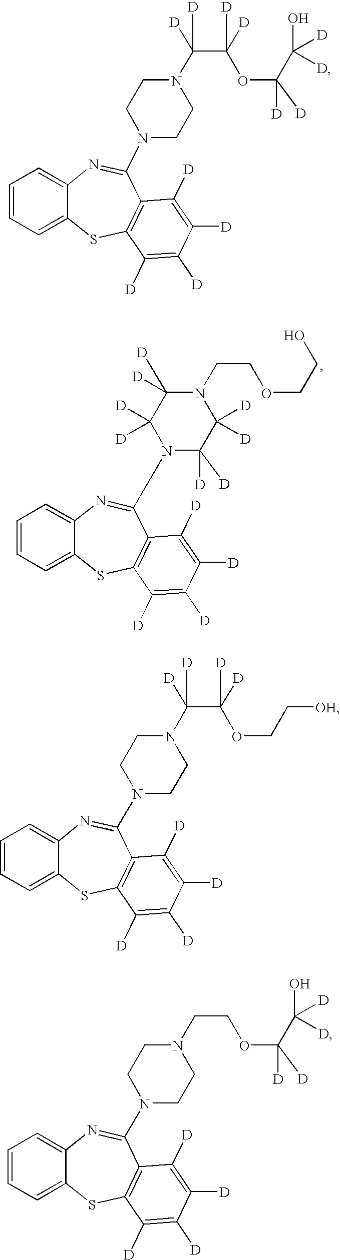

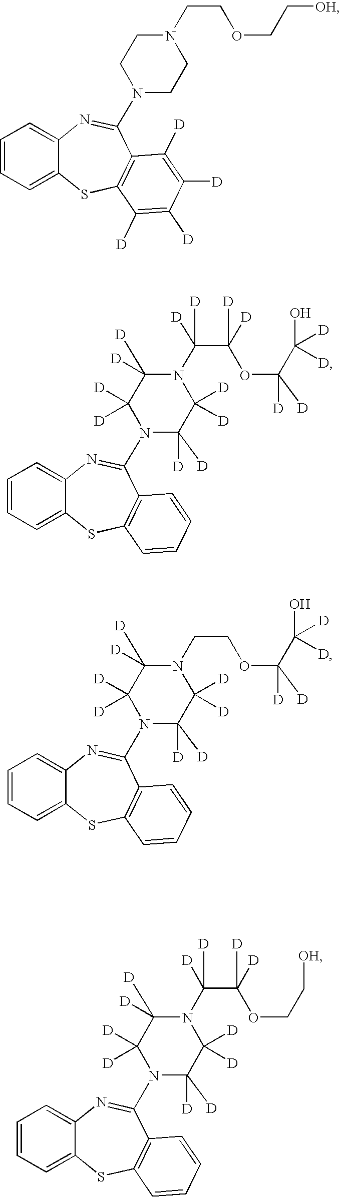

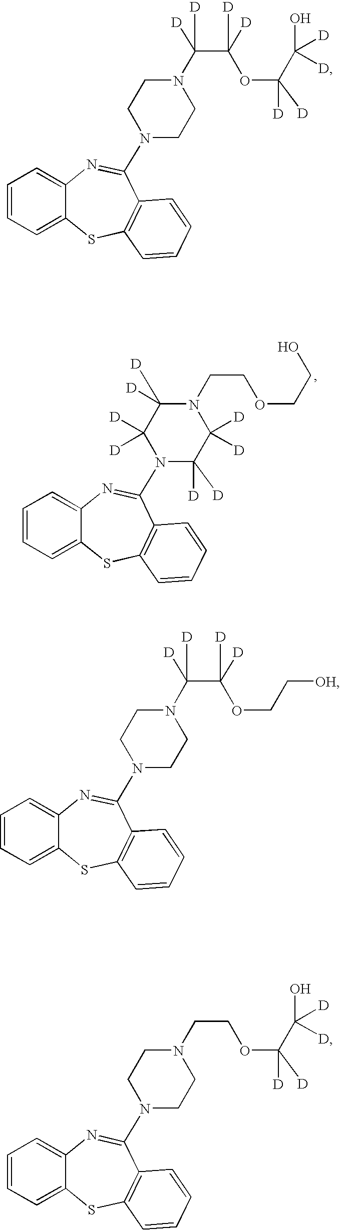

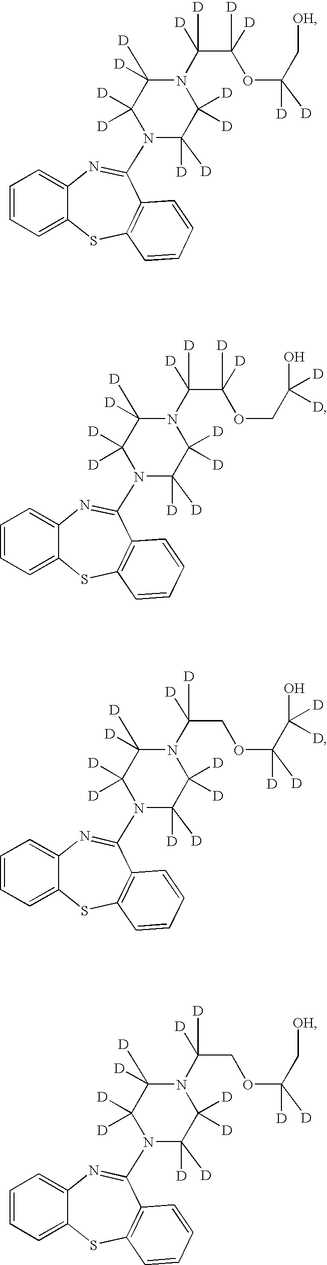

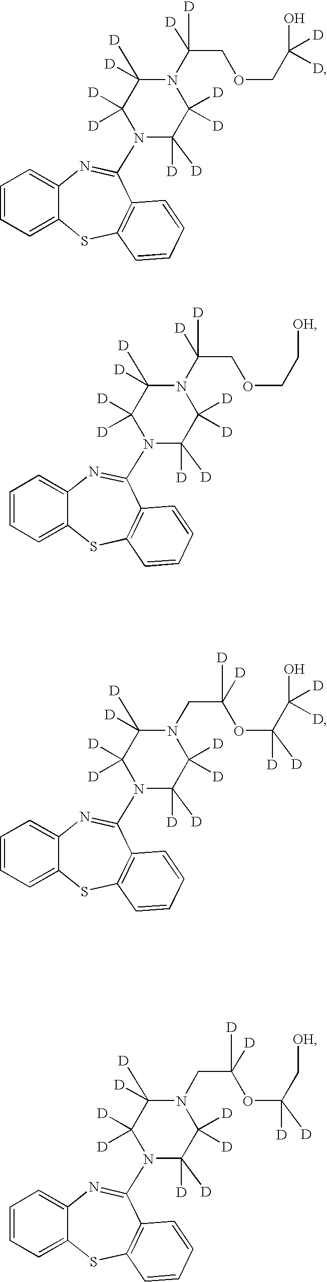

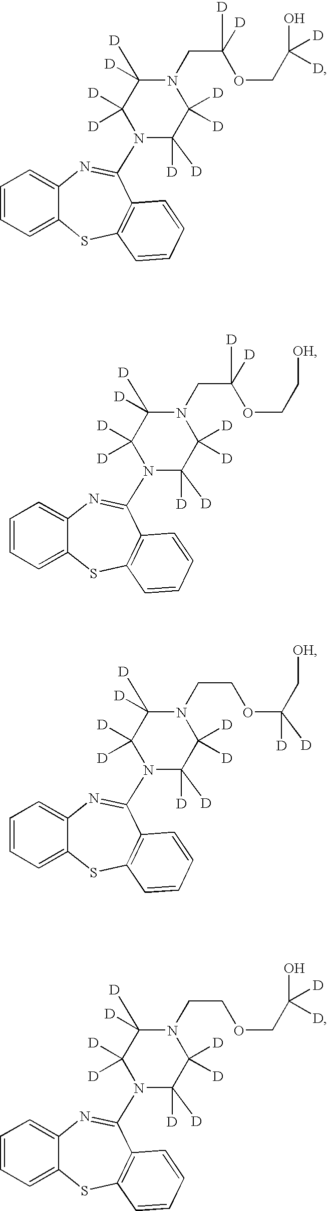

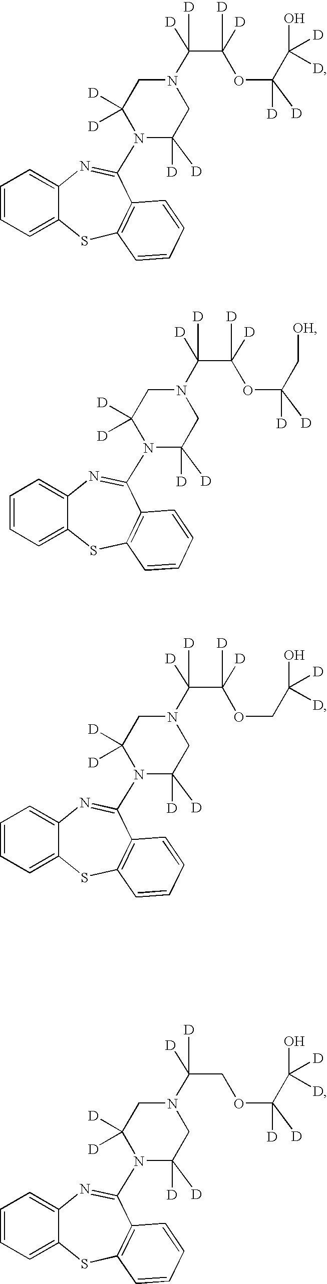

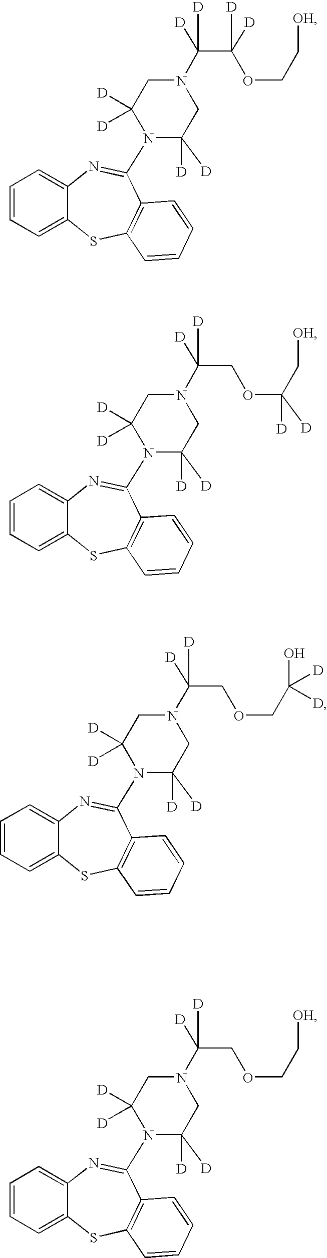

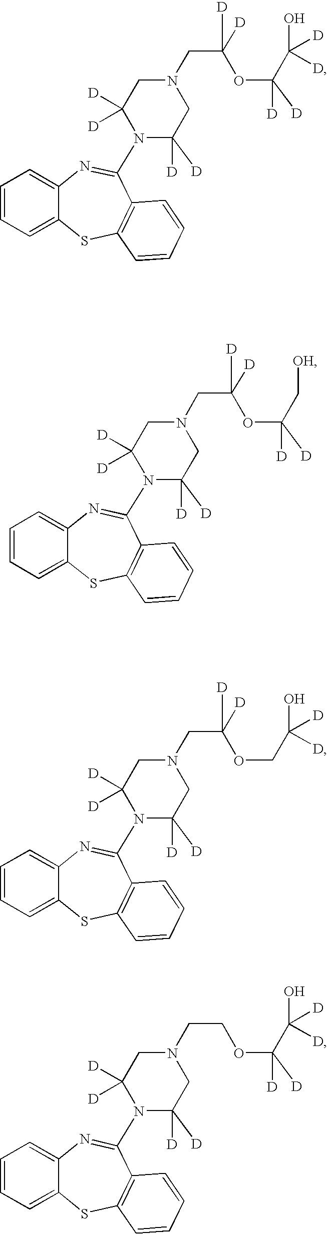

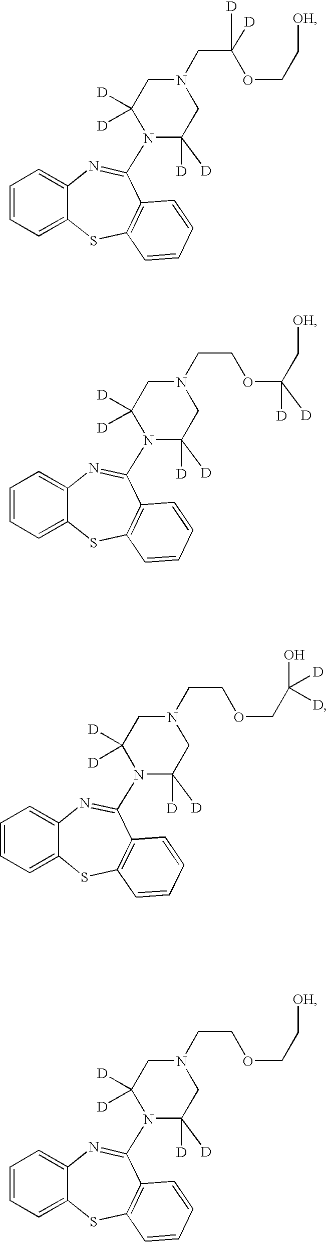

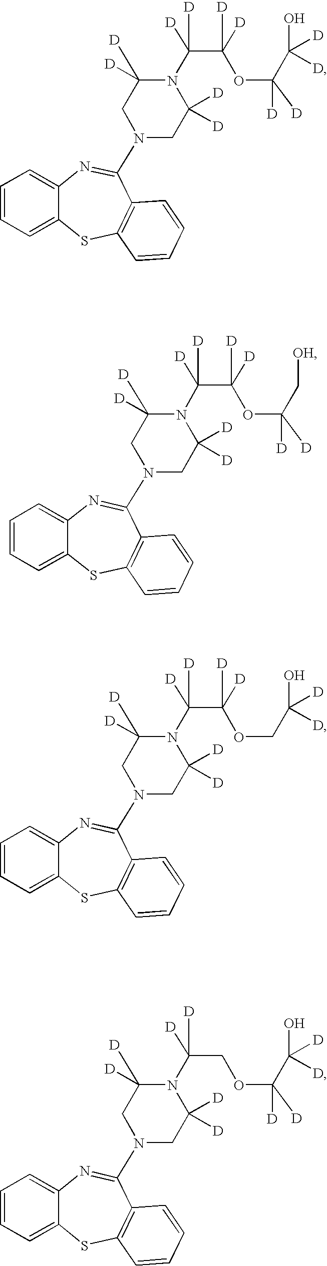

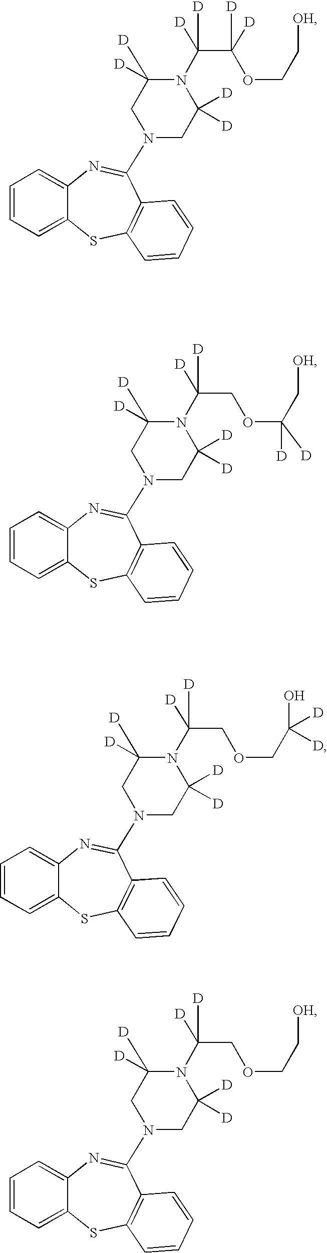

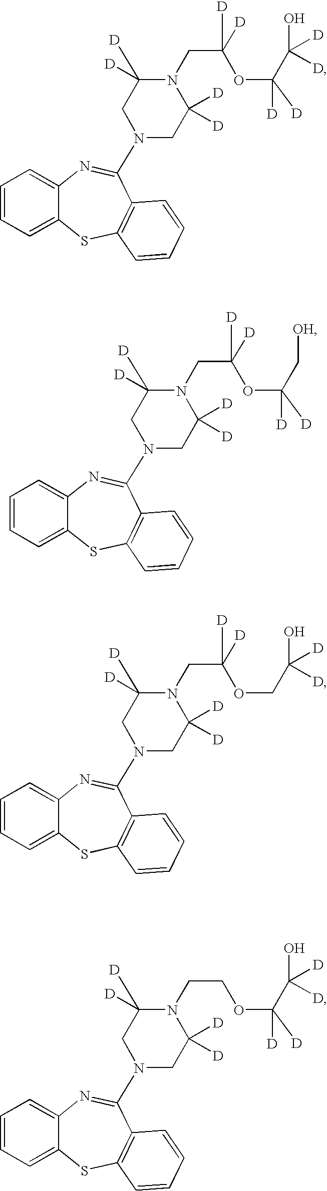

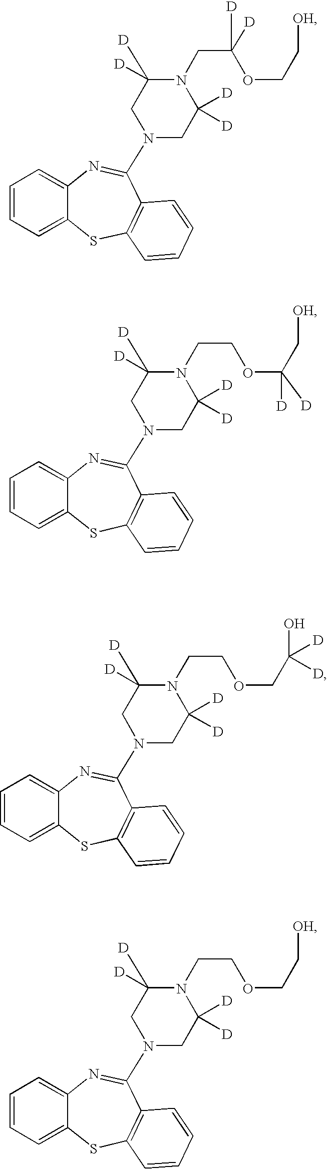

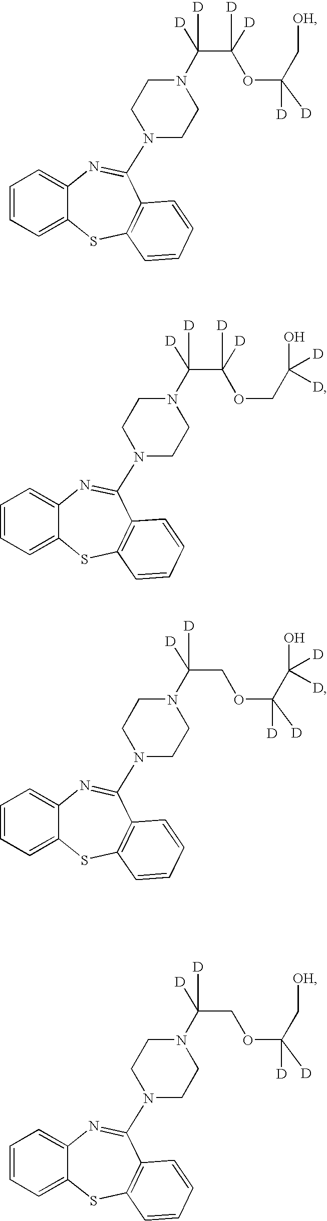

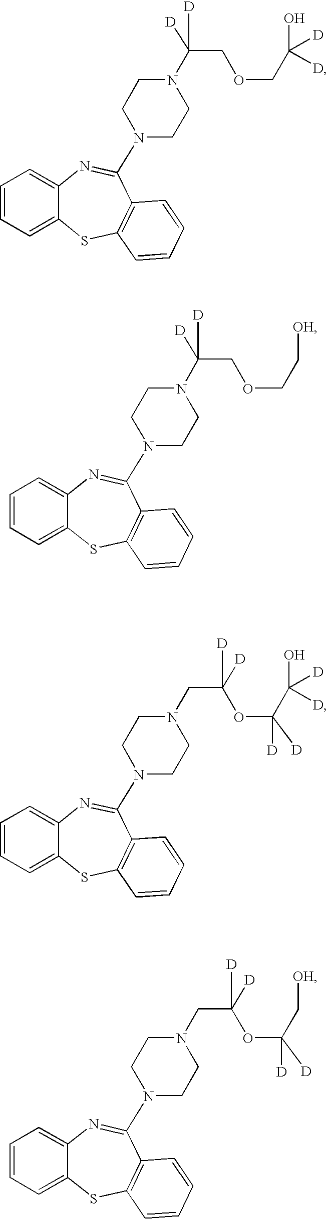

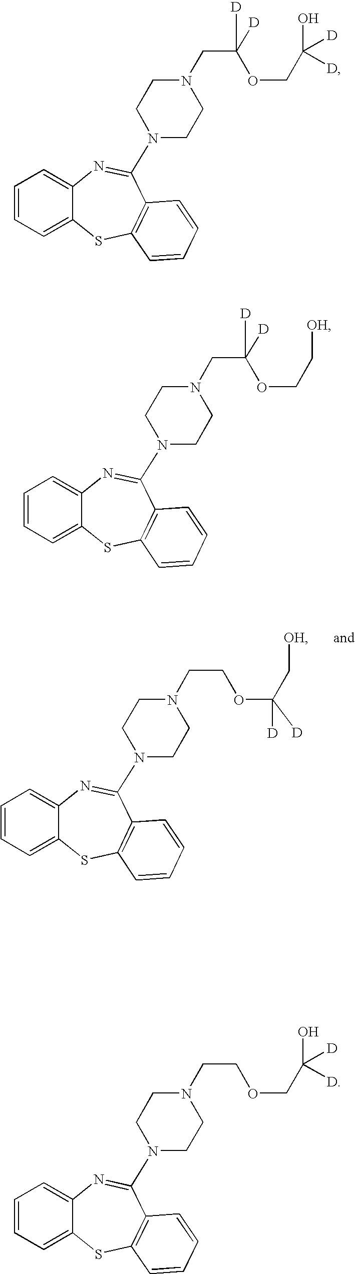

7. The compound as recited in claim 1 wherein said compound has a structural formula selected from the group consisting of:

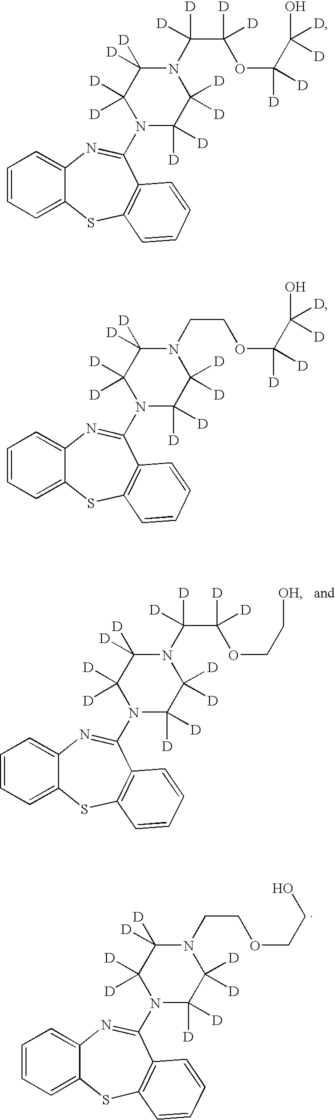

8. The compound as recited in claim 7 wherein each position represented as D has deuterium enrichment of no less than about 10%.

9. The compound as recited in claim 7 wherein each position represented as D has deuterium enrichment of no less than about 50%.

10. The compound as recited in claim 7 wherein each position represented as D has deuterium enrichment of no less than about 90%.

11. The compound as recited in claim 7 wherein each position represented as D has deuterium enrichment of no less than about 98%.

12. A pharmaceutical composition comprising a pharmaceutically acceptable carrier together with a compound of structural Formula I 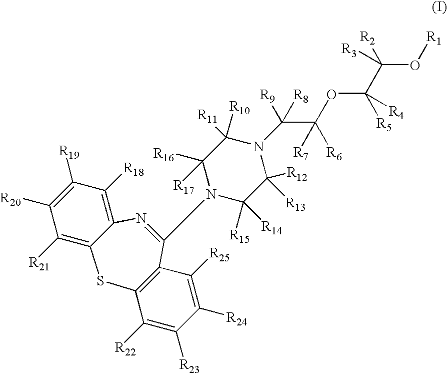
or a salt, solvate, or prodrug thereof, wherein:
R₁-R₂₅ are independently selected from the group consisting of hydrogen and deuterium; and
at least one of R₁-R₂₅ is deuterium.

13. A method of treatment of a D1 receptor-mediated disorder, a D2 receptor-mediated disorder, an alpha-1 adrenergic receptor-mediated disorder, an alpha-2 adrenergic receptor-mediated disorder, a H1 receptor-mediated disorder, a 5-HT1A receptor-mediated disorder, or a 5-HT2 receptor-mediated disorder comprising the administration, to a patient in need thereof, of a therapeutically effective amount of a compound of structural Formula I 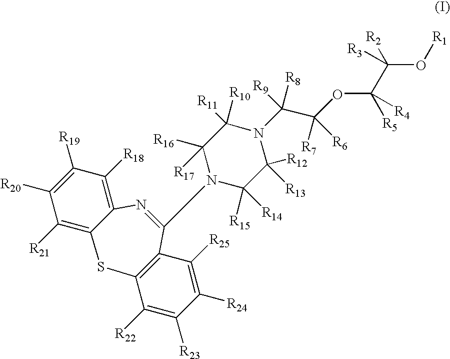
or a salt, solvate, or prodrug thereof, wherein:
R₁-R₂₅ are independently selected from the group consisting of hydrogen and deuterium; and
at least one of R₁-R₂₅ is deuterium.

14. The method as recited in claim 13 wherein said disorder is selected from the group consisting of schizophrenia, schizoaffective disorders, mania (manic disorder), bipolar I disorder, bipolar II disorder, depression associated with bipolar disorders, unipolar depression, Alzheimer's disease, dementia, Parkinson's disease, alcoholism, substance-related disorders, generalized agitation, generalized anxiety, anxiety disorders, anxiety neuroses, major depression (major depressive disorder), borderline personality disorder, post-traumatic stress disorder, primary insomnia, anorexia nervosa, social phobia, manic-depressive psychoses, mood disorders, psychotic disorders, psychosis, fibromyalgia, Tourette's syndrome and obsessive-compulsive disorder.

15. The method as recited in claim 13 further comprising the administration of an additional therapeutic agent.

16. The method as recited in claim 15 wherein said additional therapeutic agent is selected from the group consisting of antidepressants and antipsychotics.

17. The method as recited in claim 15 wherein said additional therapeutic agent is an antidepressant selected from the group consisting of citalopram, escitalopram, paroxetine, fluotexine, fluvoxamine, sertraline, isocarboxazid, moclobemide, phenelzine, tranylcypromine, amitriptyline, clomipramine, desipramine, dosulepin, imipramine, nortriptyline, protriptyline, trimipramine, lofepramine, maprotiline, amoxapine, mianserin, mirtazapine, duloxetine, nefazodone, reboxetine, trazodone, venlafaxine, tianeptine, and milnacipran.

18. The method as recited in claim 15 wherein said additional therapeutic agent is an antipsychotic selected from the group consisting of haloperidol, chlorpromazine, fluphenazine, perphenazine, prochlorperazine, thioridazine, trifluoperazine, mesoridazine, promazine, triflupromazine, levomepromazine, promethazine, chlorprothixene, flupenthixol, thiothixene, zuclopenthixol, clozapine, olanzapine, quetiapine, ziprasidone, risperidone, amisulpride, paliperidone, bifeprunox, norclozapine, aripiprazole, tetrabenazine, and cannabidiol.

19. The method as recited in claim 15 wherein said additional therapeutic agent is selected from the group consisting of lithium and valproate.

20. The method as recited in claim 13, further resulting in at least one effect selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

21. The method as recited in claim 13, further resulting in at least two effects selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

22. The method as recited in claim 13, wherein the method effects a decreased metabolism of the compound per dosage unit thereof by at least one polymorphically-expressed cytochrome P₄₅₀ isoform in the subject, as compared to the corresponding non-isotopically enriched compound.

23. The method as recited in claim 22, wherein the cytochrome P₄₅₀ isoform is selected from the group consisting of CYP2C8, CYP2C9, CYP2C19, and CYP2D6.

24. The method as recited claim 13, wherein said compound is characterized by decreased inhibition of at least one cytochrome P₄₅₀ or monoamine oxidase isoform in said subject per dosage unit thereof as compared to the non-isotopically enriched compound.

25. The method as recited in claim 24, wherein said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

26. The method as recited in claim 13, wherein the method reduces a deleterious change in a diagnostic hepatobiliary function endpoint, as compared to the corresponding non-isotopically enriched compound.

27. The method as recited in claim 26, wherein the diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

28. A compound, for use as a medicament, having structural Formula I 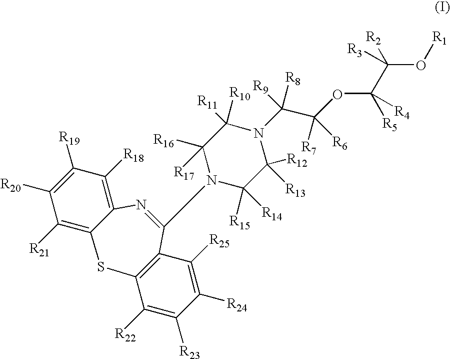
or a salt, solvate, or prodrug thereof, wherein:
R₁-R₂₅ are independently selected from the group consisting of hydrogen and deuterium; and
at least one of R₁-R₂₅ is deuterium.

29. A compound for use in the manufacture of a medicament for the prevention or treatment of a disorder ameliorated by the modulation of D1 receptors, D2 receptors, alpha-1 adrenergic receptors, alpha-2 adrenergic receptors, H1 receptors, 5-HT1A receptors, or 5-HT2 receptors, wherein said compound has structural Formula I 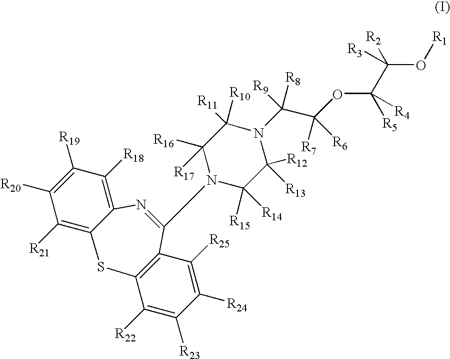
or a salt, solvate, or prodrug thereof, wherein:
R₁-R₂₅ are independently selected from the group consisting of hydrogen and deuterium; and
at least one of R₁-R₂₅ is deuterium.

30. A compound of structural formula II: 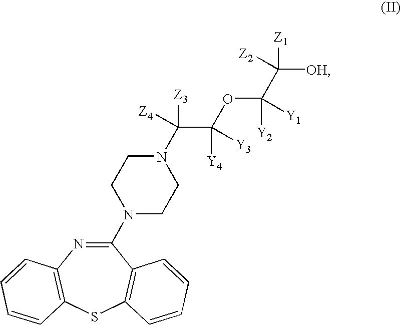
or a salt, hydrate, or solvate thereof, wherein:
each Z is independently selected from hydrogen or deuterium;
each Y is independently selected from hydrogen or deuterium; and
at least one Z is deuterium.

31. The compound of claim 30, wherein Y₁ and Y₂ are each deuterium.

32. The compound of claim 30, wherein Y₃ and Y₄ are each deuterium.

33. The compound of claim 30, wherein Z₁ and Z₂ are each deuterium.

34. The compound of claim 30, wherein Z₃ and Z₄ are each deuterium.

35. The compound of claim 30, selected from:

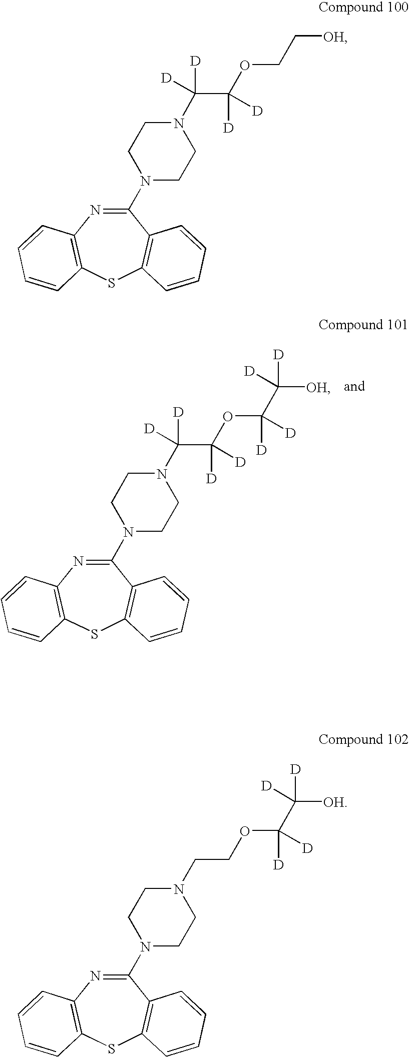

36. The compound of claim 30, wherein any atom not designated as deuterium, is present at its natural isotopic abundance.

37. A pyrogen-free composition comprising a compound of claim 30; and an acceptable carrier.

38. The composition of claim 37 formulated for pharmaceutical administration, wherein the carrier is a pharmaceutically acceptable carrier.

39. The composition of claim 38 further comprising a second therapeutic agent useful in the treatment of a disorder selected from schizophrenia, schizoaffective disorders, mania (manic disorder), bipolar I disorder, bipolar II disorder, depression associated with bipolar disorders, unipolar depression, Alzheimer's disease, dementia, Parkinson's disease, alcoholism, substance-related disorders, generalized agitation, generalized anxiety, anxiety disorders, anxiety neuroses, major depression (major depressive disorder), borderline personality disorder, post-traumatic stress disorder, primary insomnia, anorexia nervosa, social phobia, manic-depressive psychoses, mood disorders, psychotic disorders, psychosis, fibromyalgia, Tourette's syndrome and obsessive-compulsive disorder.

40. The composition of claim 39, wherein the second therapeutic agent is selected from sabcomeline; a nicotine acetylcholine alpha 7 receptor agonist; moclobemide; brofaromine; befloxatone; toloxatone; gluoxetine; citalopram; excitalopram; fluvoxamine; sertraline; paroxetine; a dopamine D1 antagonist; zolmitriptan; divalproex; lithium; and guanfacine.

41. The composition of claim 38, wherein the second therapeutic agent is selected from divalproex and lithium.

42. A method of modulating the activity of one or more of: serotonergic 5HT1A or 5HT2 receptors, dopaminergic D1 or D2 receptor, histaminergic H1 receptors, or adrenergic α1 or α2 receptors in a cell comprising contacting the cell with a compound of claim 30.

43. A method of treating a subject suffering from or susceptible to a disorder selected from schizophrenia, schizoaffective disorders, mania (manic disorder), bipolar I disorder, bipolar II disorder, depression associated with bipolar disorders, unipolar depression, Alzheimer's disease, dementia, Parkinson's disease, alcoholism, substance-related disorders, generalized agitation, generalized anxiety, anxiety disorders, anxiety neuroses, major depression (major depressive disorder), borderline personality disorder, post-traumatic stress disorder, primary insomnia, anorexia nervosa, social phobia, manic-depressive psychoses, mood disorders, psychotic disorders, psychosis, fibromyalgia, Tourette's syndrome and obsessive-compulsive disorder, comprising the step of administering to the subject in need thereof with a composition of claim 36.

44. The method of claim 43, wherein the subject is suffering from or susceptible to schizophrenia or bipolar I disorder.

45. The method of claim 43, comprising the additional step of co-administering to the subject in need thereof a second therapeutic agent selected from sabcomeline; a nicotine acetylcholine alpha 7 receptor agonist; a serotonin/norepinephrine reuptake inhibitor; moclobemide; brofaromine; befloxatone; toloxatone; gluoxetine; citalopram; excitalopram; fluvoxamine; sertraline; paroxetine; a dopamine D1 antagonist; zolmitriptan; divalproex; lithium; and guanfacine.

46. The method of claim 45, wherein:
a. the subject is suffering from or susceptible to anxiety or anxiety disorder; and the second therapeutic agent is a SSRI or a SNRI.
b. the subject is suffering from or susceptible to Alzheimer's disease or dementia; and the second therapeutic agent is divalproex;
c. the subject is suffering from or susceptible to schizophrenia; and the second therapeutic agent is guanfacine; or
d. the subject is suffering from or susceptible to bipolar I disorder and the second therapeutic agent is selected from lithium and divalproex.

47. A deuterium-enriched compound of Formula I or a pharmaceutically acceptable salt thereof: 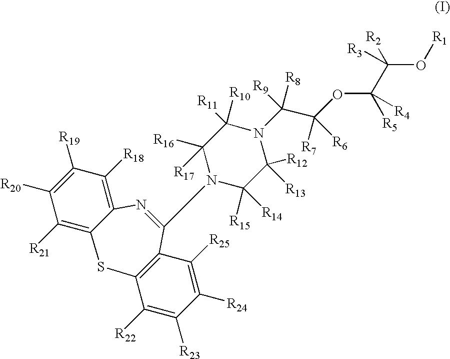
wherein R₁-R₂₅ are independently selected from H and D; and
the abundance of deuterium in R₁-R₂₅ is at least 4%.

48. A deuterium-enriched compound of claim 47, wherein the abundance of deuterium in R₁-R₂₅ is selected from the group consisting of at least 4%, at least 6%, at least 14%, at least 19%, at least 26%, at least 32%, at least 39%, at least 45%, at least 52%, at least 58%, at least 65%, at least 71%, at least 77%, at least 84%, at least 90%, at least 97%, and 100%.

49. A deuterium-enriched compound of claim 47, wherein the abundance of deuterium in R₁ is 100%.

50. A deuterium-enriched compound of claim 47, wherein the abundance of deuterium in R₂-R₅ is selected from the group consisting of at least 25%, at least 50%, at least 75%, and 100%.

51. A deuterium-enriched compound of claim 47, wherein the abundance of deuterium in R₆-R₉ is selected from the group consisting of at least 25%, at least 50%, at least 75%, and 100%.

52. A deuterium-enriched compound of claim 47, wherein the abundance of deuterium in R₁₀-R₁₃ is selected from the group consisting of at least 25%, at least 50%, at least 75%, and 100%.

53. A deuterium-enriched compound of claim 47, wherein the abundance of deuterium in R₁₄-R₁₇ is selected from the group consisting of at least 25%, at least 50%, at least 75%, and 100%.

54. A deuterium-enriched compound of claim 47, wherein the abundance of deuterium in R₁₈-R₂₅ is selected from the group consisting of at least 13%, at least 25%, at least 38%, at least 50%, at least 63%, at least 75%, at least 88%, and 100%.

55. A deuterium-enriched compound of claim 47, wherein the compound is selected from the group consisting of:

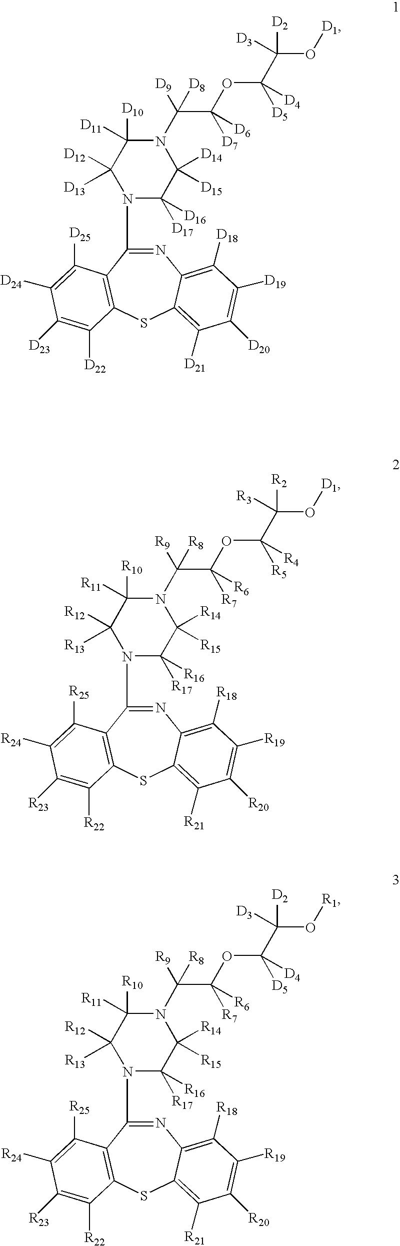

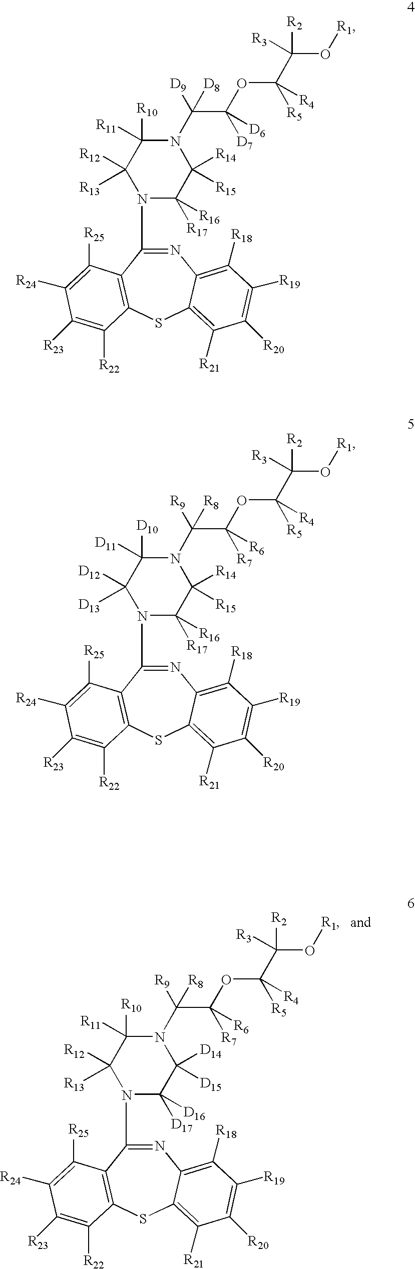

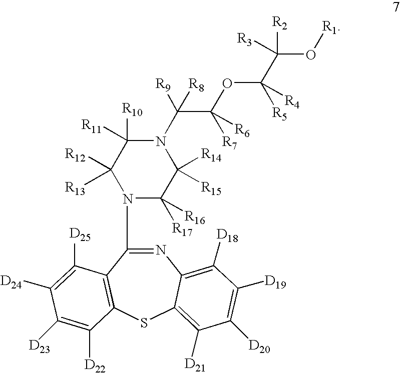

56. A deuterium-enriched compound of claim 47, wherein the compound is selected from the group consisting of:

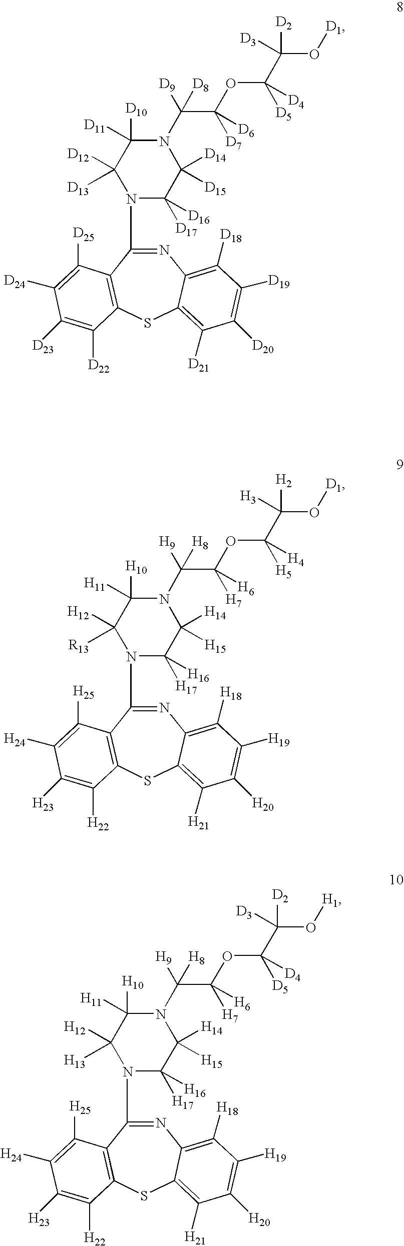

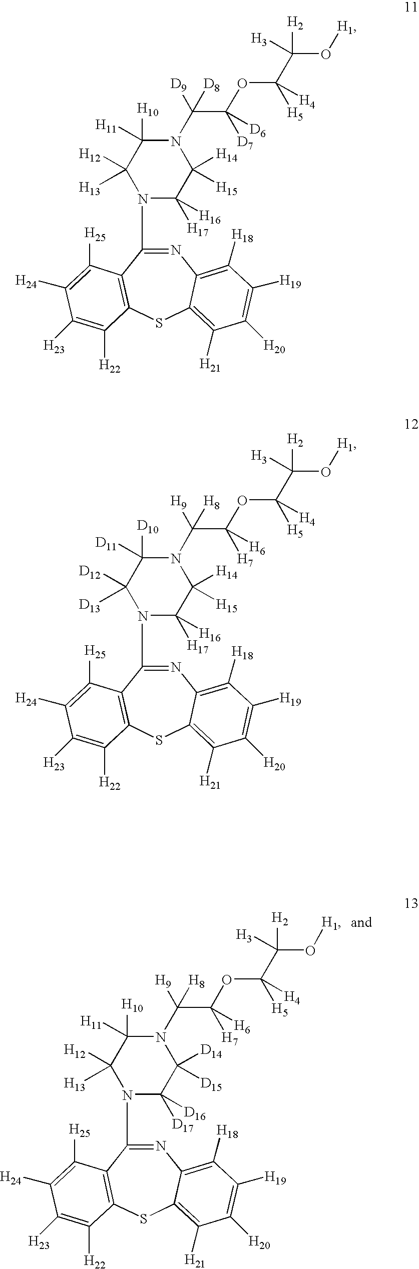

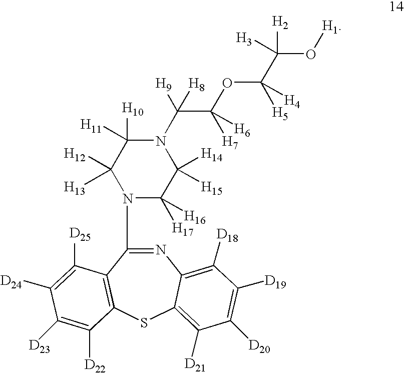

57. An isolated deuterium-enriched compound of Formula I, or a pharmaceutically acceptable salt thereof: 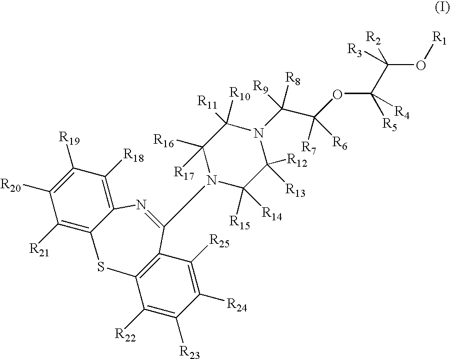
wherein R₁-R₂₅ are independently selected from H and D; and the abundance of deuterium in R₁-R₂₅ is at least 4%.

58. An isolated deuterium-enriched compound of claim 57, wherein the abundance of deuterium in R₁-R₂₅ is selected from the group consisting of at least 4%, at least 6%, at least 14%, at least 19%, at least 26%, at least 32%, at least 39%, at least 45%, at least 52%, at least 58%, at least 65%, at least 71%, at least 77%, at least 84%, at least 90%, at least 97%, and 100%.

59. An isolated deuterium-enriched compound of claim 57, wherein the abundance of deuterium in R₁ is 100%.

60. An isolated deuterium-enriched compound of claim 57, wherein the compound is compound is selected from the group consisting of:

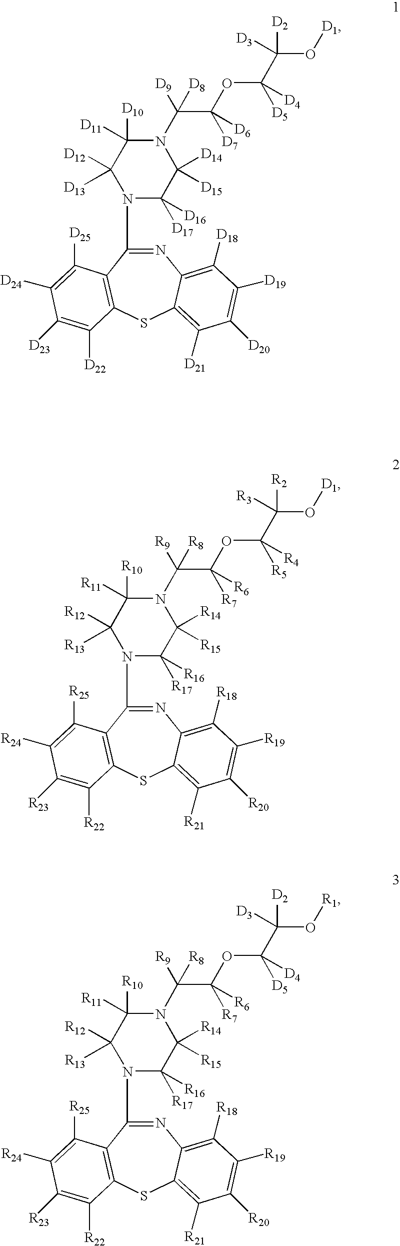

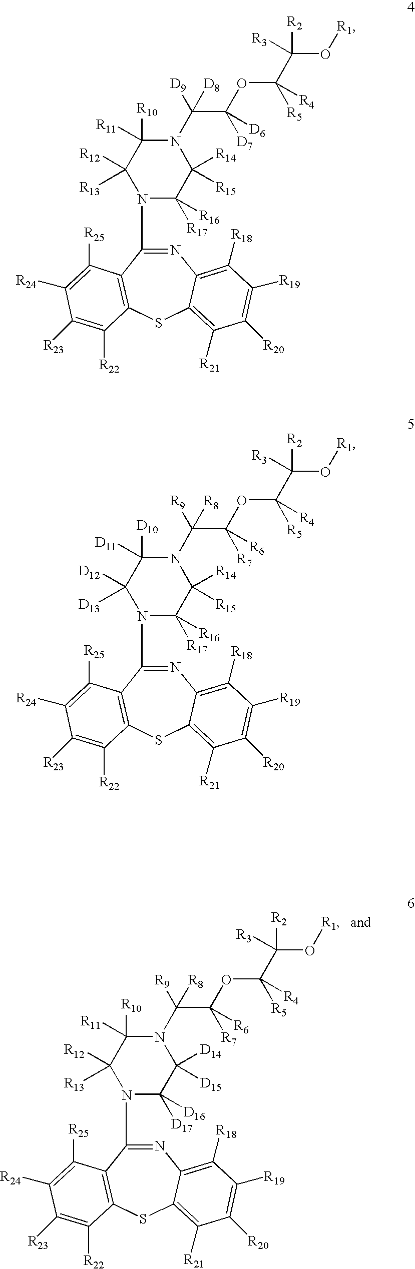

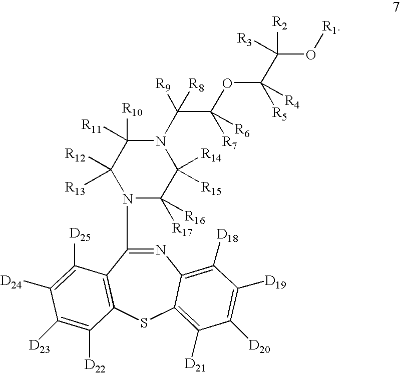

61. An isolated deuterium-enriched compound of claim 57, wherein the compound is compound is selected from the group consisting of:

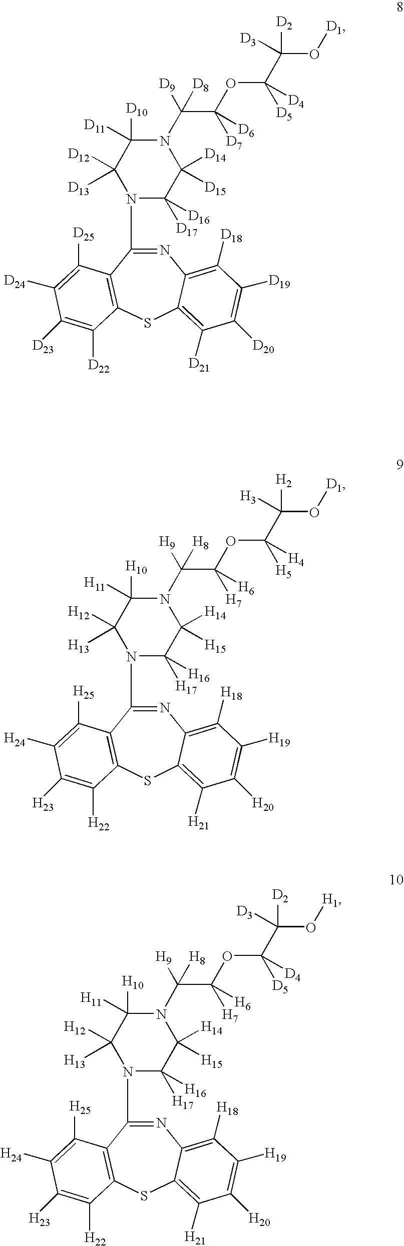

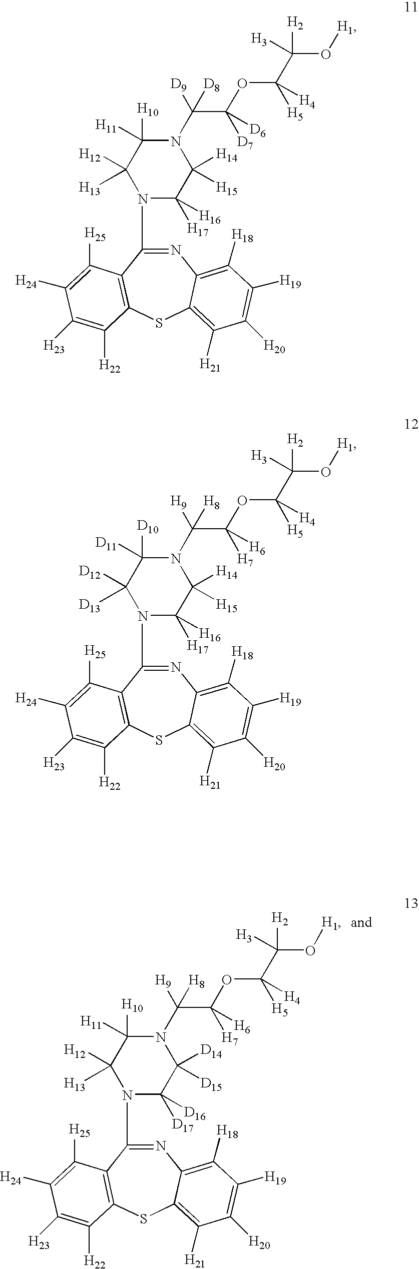

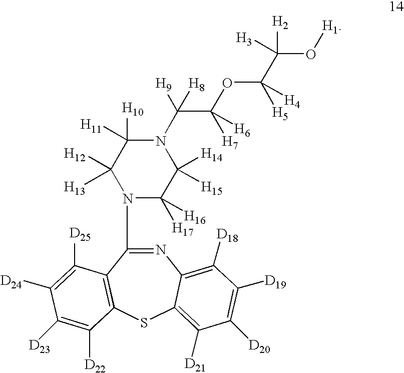

62. A mixture of deuterium-enriched compounds of Formula I, or a pharmaceutically acceptable salt thereof: 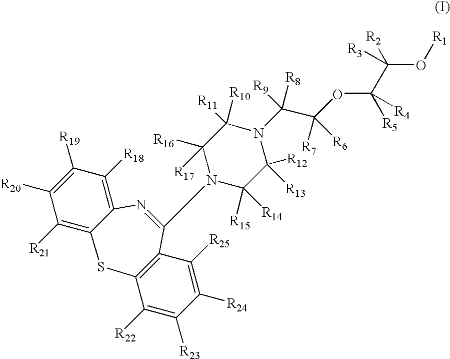
wherein R₁-R₂₅ are independently selected from H and D; and
the abundance of deuterium in R₁-R₂₅ is at least 4%.

63. A mixture of deuterium-enriched compound of claim 62, wherein the compound is selected from the group consisting of:

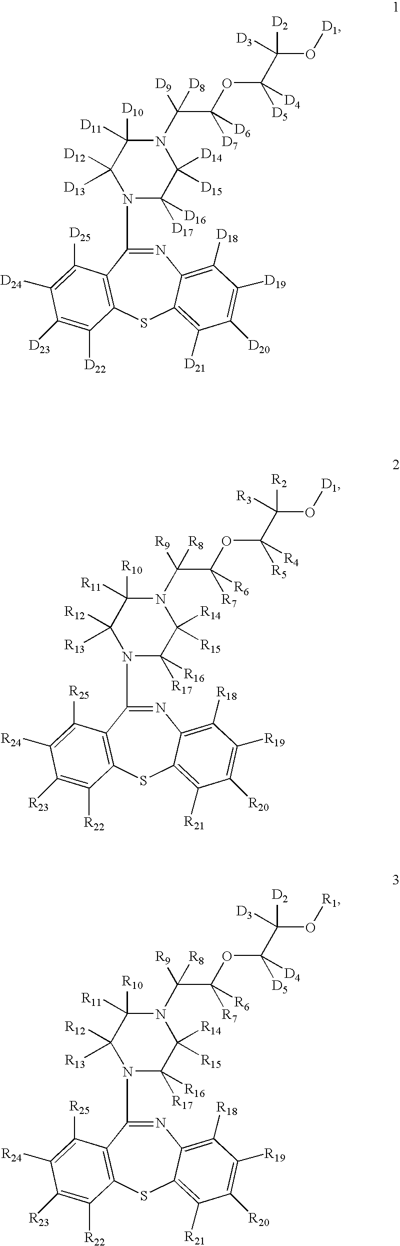

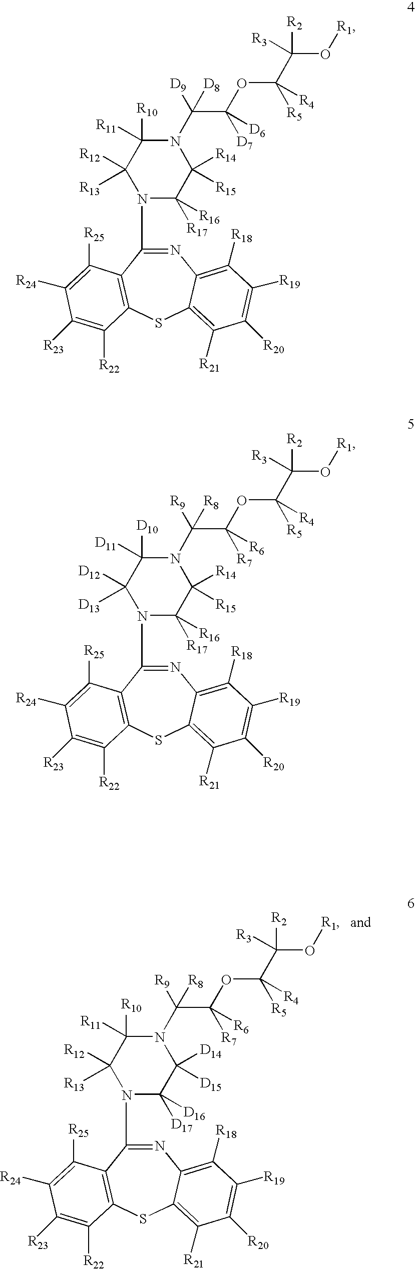

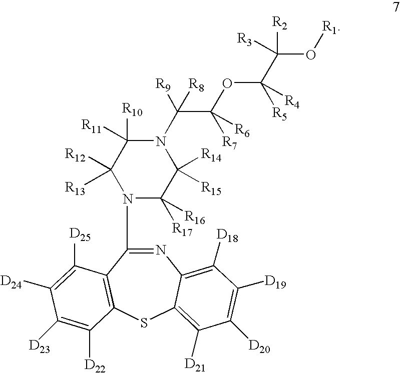

64. A mixture of deuterium-enriched compound of claim 62, wherein the compound is selected from the group consisting of:

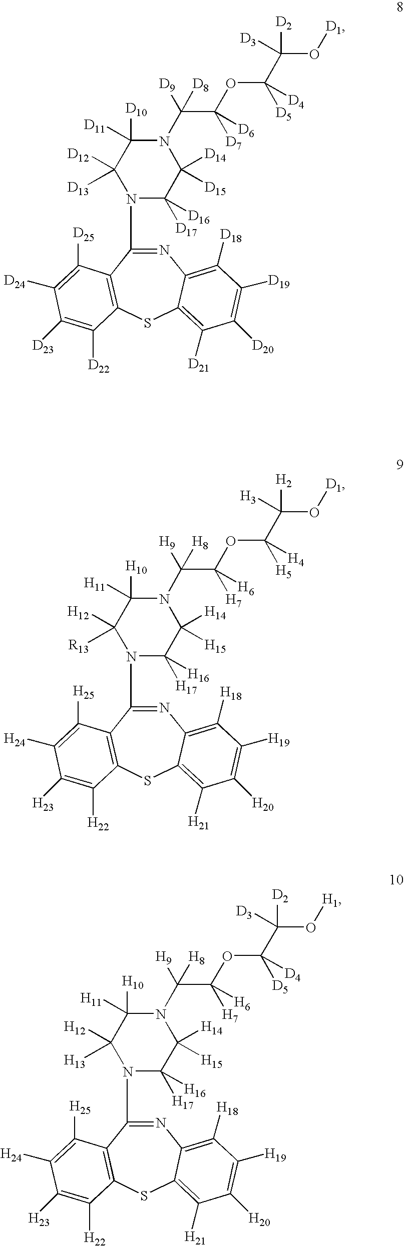

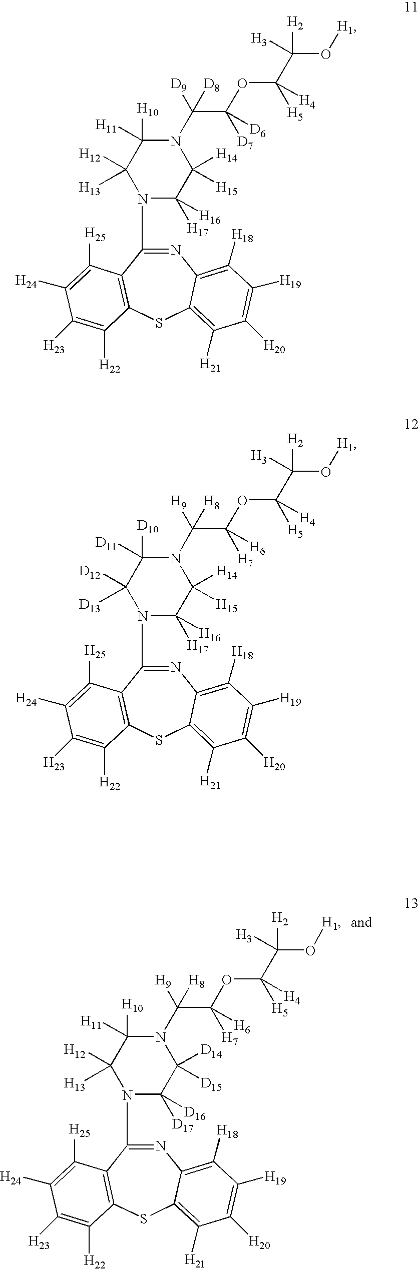

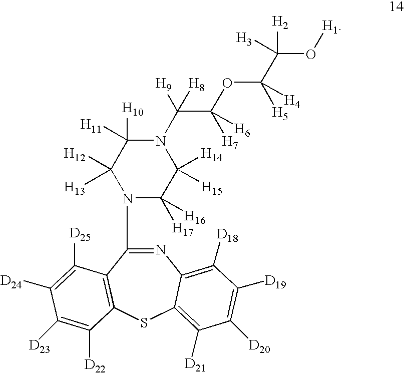

65. A pharmaceutical composition, comprising: a pharmaceutically acceptable carrier and a therapeutically effective amount of a compound of claim 47, or a pharmaceutically acceptable salt form thereof.

66. A method for treating a disease selected from schizophrenia and/or acute mania in bipolar disorder comprising: administering, to a patient in need thereof, a therapeutically effective amount of a compound of claim 47, or a pharmaceutically acceptable salt form thereof.
